(19) United States
(12) Patent Application Publication
(10) Pub. No.: US 2016/0304968 A1
(43) Pub. Date: Oct. 20, 2016

(54) Identification of Predictive Biomarkers Associated With WNT Pathway Inhibitors

(71) Applicant: ONCOMED PHARMACEUTICALS, INC., Redwood, CA (US)

(72) Inventors: ANN M. KAPOUN, Mountain View, CA (US); CHUN ZHANG, Palo Alto, CA (US)

(21) Appl. No.: 15/101,242

(22) PCT Filed: Dec. 2, 2014
(86) PCT No.: PCT/US14/68097

§ 371 Date: Jun. 2, 2016

Related U.S. Application Data
(60) Provisional application No. 61/910,663, filed on Dec. 2, 2013.
(60) Provisional application No. 61/973,339, filed on Apr. 1, 2014.

(51) Int. Cl. C12Q 1/68; C07K 14/71; A61K 39/395; A61K 47/48; A61K 38/17; C07K 16/28; A61K 31/337

(52) U.S. Cl. CPC C12Q 1/6886; C07K 16/2863; C07K 14/71; A61K 31/337; A61K 47/48284; A61K 38/179; A61K 39/39558; C12Q 2600/106; C12Q 2600/118; C12Q 2600/158; C12Q 2600/16; C07K 2317/565; C07K 2317/76; C07K 2317/56; C07K 2319/30

(57) ABSTRACT

The present invention provides biomarkers for identifying tumors likely to respond to treatment with Wnt pathway inhibitors. Also provided are methods for identifying tumors and/or patients that are likely to be responsive or non-responsive to treatment with a Wnt pathway inhibitor. Methods for treating a patient with cancer are provided, wherein the cancer is predicted to respond to a Wnt pathway inhibitor.

### CROSS-REFERENCE TO RELATED APPLICATIONS

This application claims priority benefit of U.S. Provisional Application No. 61/910,663, filed Dec. 2, 2013, and U.S. Provisional Application No. 61/975,339, filed Apr. 4, 2014, each of which are hereby incorporated by reference herein in their entirety.

### FIELD OF INVENTION

The present invention relates to the field of cancer treatment. More particularly, the invention provides methods for identifying tumors that are likely to be responsive or non-responsive to treatment with a Wnt pathway inhibitor. In addition, the invention provides methods for identifying, selecting, and/or treating patients with cancer who are likely to respond to treatment with a Wnt pathway inhibitor, either alone or in combination with other therapeutic agents.

### BACKGROUND OF THE INVENTION

Cancer is one of the leading causes of death in the developed world, with approximately 1.6 million people diagnosed with cancer and over 550,000 deaths per year in the United States alone. Overall it is estimated that more than 1 in 3 people will develop some form of cancer during their lifetime. There are more than 200 different types of cancer, four of which—breast, lung, colorectal, and prostate—account for almost half of all new cases in the United States (Siegel et al., 2012, CA: A Cancer J. for Clin., 62:10-29).

Signaling pathways normally connect extracellular signals to the nucleus leading to the expression of genes that directly or indirectly control cell growth, differentiation, survival, and death. However, in a wide variety of cancers signaling pathways are dysregulated and may be linked to tumor initiation and/or progression. Signaling pathways implicated in human oncogenesis include, but are not limited to, the Wnt pathway, the Ras-Raf-MEK-ERK or MAPK pathway, the P13K-AKT pathway, the CDKN2A/CDK4 pathway, the Bcl-2/TP53 pathway, and the NOTCH pathway.

The Wnt signaling pathway is one of several critical regulators of embryonic pattern formation, post-embryonic tissue maintenance, and stem cell biology. More specifically, Wnt signaling plays an important role in the generation of cell polarity and cell fate specification including self-renewal by stem cell populations. Unregulated activation of the Wnt pathway is associated with numerous human cancers where it is believed the activation can alter the developmental fate of cells. It is believed that the activation of the Wnt pathway may maintain tumor cells in an undifferentiated state and/or lead to uncontrolled proliferation. This may allow carcinogenesis to proceed by overtaking homeostatic mechanisms which control normal development and tissue repair (reviewed in Reya & Clevers, 2005, Nature, 434:843-50; Beachy et al., 2004, Nature, 432:324-31).

The Wnt signaling pathway was first elucidated in the Drosophila developmental mutant wingless (wg) and from the murine proto-oncogene int-1, now Wnt1(Nusse & Varmus, 1982, Cell, 31:99-109; Van Ooyen & Nusse, 1984, Cell, 39:233-40; Cabrera et al., 1987, Cell, 50:659-63; Rijsewijk et al., 1987, Cell, 50:649-57). Wnt genes encode lipid-modified glycoproteins which are secreted and 19 different Wnt proteins have been identified in mammals. These secreted ligands activate a receptor complex consisting of a Frizzled (FZD) receptor family member and low-density lipoprotein (LDL) receptor-related protein 5 or 6 (LRP5/6). The FZD receptors are members of the G-protein coupled receptor (GPCR) superfamily and contain seven transmembrane domains and a large extracellular N-terminal ligand binding domain. The N-terminal ligand binding domain contains 10 conserved cysteines and is known as a cysteine-rich domain (CRD) or a “Fri domain”. There are ten human FZD receptors, FZD1, FZD2, FZD3, FZD4, FZD5, FZD6, FZD7, FZD8, FZD9, and FZD 10. Different FZD CRDs have different binding affinities for specific Wnt proteins (Wu & Nusse, 2002, J. Biol. Chem., 277:41762-9). In addition, FZD receptors may be grouped into those that activate the canonical β-catenin pathway and those that activate non-canonical pathways (Miller et al., 1999, Oncogene, 18:7860-72).

A role for Wnt signaling in cancer was first uncovered with the identification of Wnt1(originally int1) as an oncogene in mammary tumors transformed by the nearby insertion of a murine virus (Nusse & Varmus, 1982, Cell, 31:99-109). Since these early observations additional evidence for the role of Wnt signaling in breast cancer has continued to accumulate. For example, over-expression of β-catenin in the mammary glands of transgenic mice results in hyperplasias and adenocarcinomas (Imbert et al., 2001, J. Cell Biol., 153:555-68; Michaelson & Leder, 2001, Oncogene, 20:5093-9) whereas loss of Wnt signaling disrupts normal mammary gland development (Tepera et al., 2003, J. Cell Sci., 116:1137-49; Hatsell et al., 2003, J. Mammary Gland Biol. Neoplasia, 8:145-58). In human breast cancer, β-catenin accumulation implicates activated Wnt signaling in over 50% of carcinomas, and though specific mutations have not been identified, up-regulation of Frizzled receptor expression has been observed (Brennan & Brown, 2004, J. Mammary Gland Biol. Neoplasia, 9:119-31; Malovanovic et al., 2004, Int. J. Oncol., 25:1337-42).

Activation of the Wnt pathway is also associated with colorectal cancer, lung cancer, pancreatic cancer, and melanoma. Approximately 5-10% of all colorectal cancers are hereditary with one of the main cancer types being familial adenomatous polyposis (FAP). FAP is an autosomal dominant disease in which about 80% of affected individuals contain a germline mutation in the adenomatous polyposis coli (APC) gene. Mutations have also been identified in other Wnt pathway components including Axin and β-catenin. Individual adenomas are clonal outgrowths of epithelial cells containing a second inactivated allele, and the large number of FAP adenomas inevitably results in the development of adenocarcinomas through additional mutations in oncogenes and/or tumor suppressor genes. Furthermore, activation of the Wnt signaling pathway, including loss-of-function mutations in APC and stabilizing mutations in β-catenin, can induce hyperplastic development and tumor growth in mouse models (Oshima et al., 1997, Cancer Res., 57:1644-9; Harada et al., 1999, EMBO J., 18:5931-42).

Thus the Wnt pathway has been identified as a target for cancer therapy and treatment. As drug discovery and development advances, especially in the cancer field, the “one drug fits all” approach is shifting to a “personalized medicine” strategy. Personalized medicine strategies may include treatment regimens that are based upon cancer biomarkers, including prognostic markers, pharmacodynamic markers, and predictive markers. In general, predictive biomarkers assess the likelihood that a tumor or cancer will be responsive to or sensitive to a specific therapeutic agent, and may allow for the identification and/or the selection of patients most likely to benefit from the use of that agent.

The invention provides the identification of predictive biomarkers associated with the use of Wnt pathway inhibitors in the treatment of cancer. Also provided are methods of using the predictive biomarkers for identifying, selecting, and/or classifying tumors and/or patients with cancer as likely to be responsive or non-responsive to treatment with a Wnt pathway inhibitor. Methods for treating patients with a Wnt inhibitor that are predicted to be responsive to treatment are also provided.

### SUMMARY OF THE INVENTION

Provided are biomarkers for identifying patients likely to respond to treatment with Wnt pathway inhibitors. Additionally provided are methods for identifying tumors and/or patients that are likely to be responsive or non-responsive to treatment with a Wnt pathway inhibitor. Further provided are methods of treating cancer in a patient with a Wnt pathway inhibitor, wherein the patient is predicted to be or has been identified as likely to be responsive to the Wnt pathway inhibitor.

In one aspect, the invention provides a method of identifying a human tumor that is likely to be responsive or non-responsive to treatment with a Wnt pathway inhibitor, the method comprising: (a) obtaining a sample of the human tumor; (b) measuring the expression level of each biomarker of a biomarker signature in the sample, wherein the biomarker signature comprises one or more of the biomarkers FBXW2, CCND2, RHOU, CRBP2, WIF1, and DKK1; and (c) identifying the tumor as likely to be responsive or non-responsive to treatment based upon the expression level of the biomarkers. In some embodiments, a method of identifying a human tumor that is likely to be responsive or non-responsive to treatment with a Wnt pathway inhibitor comprises: (a) obtaining a sample of the human tumor; (b) measuring the expression level of each biomarker of a biomarker signature in the sample, wherein the biomarker signature comprises one or more of the biomarkers FBXW2, CCND2, RHOU, CRBP2, WIF1, and DKK1; and (c) calculating a decision value based upon the standardized expression of the biomarkers in the biomarker signature; wherein a positive decision value indicates the tumor is predicted to be responsive to the Wnt pathway inhibitor and a negative decision value indicates the tumor is predicted to be non-responsive to the Wnt pathway inhibitor. As used herein, “standardized” and “normalized” may be used interchangeably. In some embodiments, the method comprises identifying a human tumor that is likely to be responsive or non-responsive to treatment with a Wnt pathway inhibitor in combination with paclitaxel.

In another aspect, the invention provides a method of classifying a human tumor as likely to be responsive or non-responsive to treatment with a Wnt pathway inhibitor, the method comprising: (a) obtaining a sample of the human tumor; (b) measuring the expression level of each biomarker of a biomarker signature in the sample, wherein the biomarker signature comprises one or more of the biomarkers FBXW2, CCND2, RHOU, CTBP2, WIF1, and DKK1; and (c) classifying the tumor as likely to be responsive or non-responsive to treatment based upon the expression level of the biomarkers. In some embodiments, a method of classifying a human tumor as likely to be responsive or non-responsive to treatment with a Wnt pathway inhibitor comprises: (a) obtaining a sample of the human tumor; (b) measuring the expression level of each biomarker of a biomarker signature in the sample, wherein the biomarker signature comprises one or more of the biomarkers FBXW2, CCND2, RHOU, CTBP2, WIF1, and DKK1; and (c) calculating a decision value based upon the standardized expression of the biomarkers in the biomarker signature; wherein a positive decision value indicates the tumor is predicted to be responsive to the Wnt pathway inhibitor and a negative decision value indicates the tumor is predicted to be non-responsive to the Wnt pathway inhibitor. In some embodiments, the method comprises classifying a human tumor as likely to be responsive or non-responsive to treatment with a Wnt pathway inhibitor in combination with paclitaxel.

In another aspect, the invention provides a method of determining the responsiveness (or sensitivity) of a human tumor to treatment with a Wnt pathway inhibitor, the method comprising: (a) obtaining a sample of the human tumor; (b) measuring the expression level of each biomarker of a biomarker signature in the sample, wherein the biomarker signature comprises one or more of the genes FBXW2, CCND2, RHOU, CTBP2, WIF1, and DKK1; and (c) determining the responsiveness of the tumor to treatment based upon the expression level of the biomarkers. In some embodiments, a method of determining the responsiveness or sensitivity of a human tumor to treatment with a Wnt pathway inhibitor comprises: (a) obtaining a sample of the human tumor; (b) measuring the expression level of each biomarker of a biomarker signature in the sample, wherein the biomarker signature comprises one or more of the genes FBXW2, CCND2, RHOU, CTBP2, WIF1, and DKK1; and (c) calculating a decision value based upon the standardized expression of the biomarkers in the biomarker signature; wherein a positive decision value indicates the tumor is predicted to be responsive to or sensitive to the Wnt pathway inhibitor. In some embodiments, the method comprises determining the responsiveness or sensitivity of a human tumor to treatment with a Wnt pathway inhibitor in combination with paclitaxel.

In another aspect, the invention provides a method of identifying a patient with cancer who is likely to respond to treatment with a Wnt pathway inhibitor, the method comprising: (a) obtaining a sample of the patient's tumor; (b) measuring the expression level of each biomarker of a biomarker signature in the sample, wherein the biomarker signature comprises one or more of the biomarkers FBXW2, CCND2, RHOU, CTBP2, WIF1, and DKK1; and (c) identifying the patient who is likely to respond to treatment based upon the expression level of the biomarkers. In some embodiments, a method of identifying a patient with cancer who is likely to respond to treatment with a Wnt pathway inhibitor comprises: (a) obtaining a sample of the patient's tumor; (b) measuring the expression level of each biomarker of a biomarker signature in the sample, wherein the biomarker signature comprises one or more of the biomarkers FBXW2, CCND2, RHOU, CTBP2, WIF1, and DKK1; and (c) calculating a decision value based upon the standardized expression of the biomarkers in the biomarker signature; wherein a positive decision value indicates that the patient is predicted to respond to treatment with the Wnt pathway inhibitor. In some embodiments, the method comprises identifying a patient with cancer who is likely to respond to treatment with a Wnt pathway inhibitor in combination with paclitaxel.

In another aspect, the invention provides a method of selecting a patient with cancer for treatment with a Wnt pathway inhibitor, the method comprising: (a) obtaining a sample of the patient's tumor; (b) measuring the expression level of each biomarker of a biomarker signature in the sample, wherein the biomarker signature comprises one or more of the biomarkers FBXW2, CCND2, RHOU, CTBP2, WIF 1, and DKK1; (c) selecting the patient for treatment based upon the expression level of the biomarkers. In some embodiments, a method of selecting a patient with cancer for treatment with a Wnt pathway inhibitor comprises: (a) obtaining a sample of the patient's tumor; (b) measuring the expression level of each biomarker of a biomarker signature in the sample, wherein the biomarker signature comprises one or more of the biomarkers FBXW2, CCND2, RHOU, CTBP2, WIF1, and DKK1; (c) calculating a decision value based upon the standardized expression of the biomarkers in the biomarker signature; and (d) selecting the patient for treatment when their tumor sample has a positive decision value. In some embodiments, the method comprises selecting a patient with cancer for treatment with a Wnt pathway inhibitor in combination with paclitaxel.

In another aspect, the invention provides a method of treating cancer in a patient, comprising: (a) identifying if the patient is likely to respond to treatment with a Wnt pathway inhibitor, wherein the identification comprises: (i) obtaining a sample of the patient's cancer; (ii) measuring the expression level of each biomarker of a biomarker signature in the sample, wherein the biomarker signature comprises one or more of the biomarkers FBXW2, CCND2, RHOU, CTBP2, WIF1, and DKK1; and (iii) identifying the patient who is likely to respond to treatment based upon the expression level of the biomarkers; and (b) administering to the patient who is likely to response to treatment an effective amount of the Wnt pathway inhibitor. In some embodiments, a method of treating cancer in a patient comprises: (a) identifying if the patient is likely to respond to treatment with a Wnt pathway inhibitor, wherein the identification comprises: (i) obtaining a sample of the patient's cancer; (ii) measuring the expression level of each biomarker of a biomarker signature in the sample, wherein the biomarker signature comprises one or more of the biomarkers FBXW2, CCND2, RHOU, CTBP2, WIF1, and DKK1; and (iii) calculating a decision value based upon the standardized expression of the biomarkers in the signature; wherein a positive decision value indicates that a patient is predicted to respond to treatment; and (b) administering to the patient who is predicted to response to treatment an effective amount of the Wnt pathway inhibitor. In some embodiments, the method comprises identifying if the patient is likely to respond to treatment with a Wnt pathway inhibitor in combination with paclitaxel. In some embodiments, the method comprises administering to the patient the Wnt pathway inhibitor in combination with paclitaxel.

In another aspect, the invention provides a method of treating cancer in a patient, comprising: administering an effective amount of a Wnt pathway inhibitor to the patient; wherein the patient is predicted to respond to treatment with a Wnt inhibitor based upon expression levels of a biomarker signature in a patient tumor sample, wherein the signature comprises one or more of the biomarkers FBXW2, CCND2, RHOU, CTBP2, WIF1, and DKK1. In some embodiments, a method of treating cancer in a patient comprises: administering an effective amount of a Wnt pathway inhibitor to the patient; wherein the patient is predicted to respond to treatment based upon a positive decision value calculated from the weighted sum of the standardized expression of biomarkers in a biomarker signature in a patient tumor sample, wherein the set of biomarkers comprises one or more of the biomarkers FBXW2, CCND2, RHOU, CTBP2, WIF1, and DKK1. In some embodiments, the patient is predicted to respond to treatment with a Wnt pathway inhibitor in combination with paclitaxel. In some embodiments, the method comprises administering to the patient the Wnt pathway inhibitor in combination with paclitaxel.

In another aspect, the invention provides a method for increasing the likelihood of effective treatment with a Wnt pathway inhibitor, comprising: (a) identifying if a patient has a tumor that is likely to respond to treatment with a Wnt pathway inhibitor, wherein the identification comprises: (i) obtaining a sample of the patient's cancer; (ii) measuring the expression level of each biomarker of a biomarker signature in the sample, wherein the biomarker signature comprises one or more of the biomarkers FBXW2, CCND2, RHOU, CTBP2, WIF1, and DKK1; and (iii) identifying the patient who is likely to respond to treatment based upon the expression level of the biomarkers; and (b) administering an effective amount of the Wnt pathway inhibitor to the patient. In some embodiments, a method for increasing the likelihood of effective treatment with a Wnt pathway inhibitor comprises: (a) identifying if a patient has a tumor that is likely to respond to treatment with a Wnt pathway inhibitor, wherein the identification comprises: (i) obtaining a sample of the patient's cancer; (ii) measuring the expression level of each biomarker of a biomarker signature in the sample, wherein the biomarker signature comprises one or more of the biomarkers FBXW2, CCND2, RHOU, CTBP2, WIF1, and DKK1; and (iii) calculating a decision value based upon the standardized expression of the biomarkers in the biomarker signature; wherein a positive decision value indicates that a patient is predicted to respond to treatment; and (b) administering an effective amount of the Wnt pathway inhibitor to the patient whose tumor has a positive decision value. In some embodiments, the method comprises identifying if a patient has a tumor that is likely to respond to treatment with a Wnt pathway inhibitor in combination with paclitaxel. In some embodiments, the method comprises administering to the patient the Wnt pathway inhibitor in combination with paclitaxel.

In another aspect, the invention provides a method for increasing the likelihood of effective treatment with a Wnt pathway inhibitor, comprising: administering an effective amount of a Wnt pathway inhibitor to a patient; wherein the patient is identified as likely to respond to treatment with a Wnt inhibitor based upon expression levels of a biomarker signature in a patient tumor sample, wherein the signature comprises one or more of the biomarkers FBXW2, CCND2, RHOU, CTBP2, WIF1, and DKK1. In some embodiments, a method for increasing the likelihood of effective treatment with a Wnt pathway inhibitor comprises: administering an effective amount of a Wnt pathway inhibitor to a patient; wherein the patient is identified as likely to respond to treatment based upon a positive decision value calculated from the weighted sum of the standardized expression of biomarkers in a biomarker signature in a patient tumor sample, wherein the set of biomarkers comprises one or more of the biomarkers FBXW2, CCND2, RHOU, CTBP2, WIF1, and DKK1. In some embodiments, the patient is identified as likely to respond to treatment with a Wnt pathway inhibitor in combination with paclitaxel. In some embodiments, the method comprises administering to the patient the Wnt pathway inhibitor in combination with paclitaxel.

In certain embodiments of each of the aforementioned aspects, as well as other aspects and/or embodiments described elsewhere herein, the biomarker signature comprises one or more of the biomarkers FBXW2, CCND2, RHOU, CTBP2, WIF1, DKK1, EP300, and CTBP1. In some embodiments, the biomarker signature comprises one or more of the biomarkers FBXW2, CCND2, RHOU, CTBP2, WIF1, DKK1, EP300, CTBP1, WNT6, WNT3, FZD2, APC, TLE2, DVL2, PITX2, WISP1, GSK3B, WNT9A, FZD7, and LEF1. In some embodiments, the biomarker signature comprises one or more of the biomarkers FBXW2, CCND2, RHOU, CTBP2, WIF1, and DKK1, and at least one additional biomarker from Table 2.

In certain embodiments of each of the aforementioned aspects, as well as other aspects and/or embodiments described elsewhere herein, the Wnt pathway inhibitor is an antibody. In some embodiments, the Wnt pathway inhibitor is an antibody that specifically binds at least one Frizzled (FZD) protein or fragment thereof. In some embodiments, the Wnt pathway inhibitor is an antibody that specifically binds at least one FZD protein selected from the group consisting of: FZD 1, FZD2, FZD3, FZD4, FZD5, FZD6, FZD7, FZD8, FZD9, and FZD10. In some embodiments, the Wnt pathway inhibitor is an antibody that specifically binds at least one FZD protein selected from the group consisting of: FZD1, FZD2, FZD5, FZD7, and FZD8. In certain embodiments, the Wnt pathway inhibitor is an antibody which comprises: (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6).

In certain embodiments, the Wnt pathway inhibitor is an antibody which comprises a heavy chain variable region comprising SEQ ID NO:7 and a light chain variable region comprising SEQ ID NO:8. In certain embodiments, the Wnt pathway inhibitor is an antibody which comprises a heavy chain variable region and a light chain variable region encoded by the plasmid deposited with ATCC as PTA-9541. In certain embodiments, the Wnt pathway inhibitor is an antibody which comprises a heavy chain and a light chain encoded by the plasmid deposited with ATCC as PTA-9541. In some embodiments, the Wnt pathway inhibitor is antibody OMP-18R5.

In certain embodiments of each of the aforementioned aspects, as well as other aspects and/or embodiments described elsewhere herein, the Wnt pathway inhibitor is a soluble receptor. In some embodiments, the Wnt pathway inhibitor comprises the extracellular domain of a FZD receptor protein. In some embodiments, the Wnt pathway inhibitor comprises a Fri domain of a FZD protein. In some embodiments, the Wnt pathway inhibitor comprises the Fri domain of FZD8. In certain embodiments, the Wnt pathway inhibitor comprises the Fri domain of FZD8 and a human Fc domain. In some embodiments, the Wnt pathway inhibitor is the soluble receptor OMP-54F28.

In some embodiments, the tumor is selected from the group consisting of a breast tumor, lung tumor, a colon tumor, glioma, a gastrointestinal tumor, a renal tumor, an ovarian tumor, a liver tumor, a colorectal tumor, an endometrial tumor, a kidney tumor, a prostate tumor, a thyroid tumor, a neuroblastoma, a pancreatic tumor, a glioblastoma multiforme, a cervical tumor, a stomach tumor, a bladder tumor, a hepatoma, melanoma, and a head and neck tumor. In some embodiments, the tumor is a breast tumor.

In some embodiments, the cancer is selected from the group consisting of a breast cancer, lung cancer, a colon cancer, glioma, a gastrointestinal cancer, a renal cancer, an ovarian cancer, a liver cancer, a colorectal cancer, an endometrial cancer, a kidney cancer, a prostate cancer, a thyroid cancer, a neuroblastoma, a pancreatic cancer, a glioblastoma multiforme, a cervical cancer, a stomach cancer, a bladder cancer, a hepatoma, melanoma, and a head and neck cancer. In some embodiments, the cancer is breast cancer.

In some embodiments, the method further comprises administering a second therapeutic agent to the patient. In some embodiments, the second therapeutic agent is a chemotherapeutic agent. In some embodiments, the second therapeutic agent is paclitaxel.

In certain embodiments of each of the aforementioned aspects, as well as other aspects and/or embodiments described elsewhere herein, the sample includes, but is not limited to, any clinically relevant tissue sample, such as a tumor biopsy, a core biopsy tissue sample, a fine needle aspirate, a hair follicle, or a sample of bodily fluid, such as blood, plasma, serum, lymph, ascitic fluid, cystic fluid, or urine. In some embodiments, the sample is taken from a patient having a tumor or cancer. In some embodiments, the sample is a primary tumor. In some embodiments, the sample is a metastasis. In some embodiments, the sample is a tissue sample. In some embodiments, the sample is a tumor sample. In some embodiments, the sample is a fresh frozen (FF) tissue sample. In some embodiments, the sample is a formalin-fixed paraffin embedded (FFPE) tissue sample. In some embodiments, the sample is whole blood, plasma, or serum. In some embodiments, the sample is cells. In some embodiments, the sample is circulating tumor cells (CTCs).

In certain embodiments of each of the aforementioned aspects, as well as other aspects and/or embodiments described elsewhere herein, the expression level of a biomarker is determined using PCR-based methods, such as but not limited to, reverse transcription PCR (RT-PCR), quantitative RT-PCR (qPCR), TaqMan™, or TaqMan™ low density array (TLDA). In some embodiments, the expression level of a biomarker is determined using a microarray.

In certain embodiments of each of the aforementioned aspects, as well as other aspects and/or embodiments described elsewhere herein, the standardized expression of each biomarker is determined by measuring an expression level for each biomarker and multiplying it by a corresponding weight, wherein the weight for each biomarker is determined by the biomarker expression. In certain embodiments, the decision value is calculated according to the equation: 0.4560427*FBXW2+0.3378467*CCND2−0.4809354*RHOU+0.409029*CTBP2+0.3291529*WIF1+0.2926374*DKK1+0.04662682.

In some embodiments, the expression level of a biomarker is measured or determined by a PCR-based assay. In some embodiments, the expression levels of FBXW2, CCND2, RHOU, CTBP2, WIF1, and DKK1 are measured using polynucleotides selected from the group consisting of SEQ ID NOs:62-79. In some embodiments, the expression levels of FBXW2, CCND2, RHOU, CTBP2, WIF1, and DKK1 are measured using (a) a forward primer of SEQ ID NO:62, a reverse primer of SEQ ID NO:63, and a probe comprising SEQ ID NO:64; (b) a forward primer of SEQ ID NO:65, a reverse primer of SEQ ID NO:66, and a probe comprising SEQ ID NO:67; (c) a forward primer of SEQ ID NO:68, a reverse primer of SEQ ID NO:69, and a probe comprising SEQ ID NO:70; (d) a forward primer of SEQ ID NO:71, a reverse primer of SEQ ID NO:72, and a probe comprising SEQ ID NO:73; (e) a forward primer of SEQ ID NO:74, a reverse primer of SEQ ID NO:75, and a probe comprising SEQ ID NO:76; and (f) a forward primer of SEQ ID NO:77, a reverse primer of SEQ ID NO:78, and a probe comprising SEQ ID NO:79.

In some embodiments, the expression level of a biomarker is measured or determined by multi-analyte profile testing, radioimmunoassay (RIA), Western blot assay, immunofluorescent assay, enzyme immunoassay, enzyme linked immunosorbent assay (ELISA), immunoprecipitation assay, chemiluminescent assay, immunohistochemical assay, dot blot assay, or slot blot assay. In some embodiments wherein the assay uses an antibody, the antibody is detectably labeled. In some embodiments, the label is selected from the group consisting of an immunofluorescent label, a chemiluminescent label, a phosphorescent label, an enzyme label, a radiolabel, an avidin/biotin label, colloidal gold particles, colored particles, and magnetic particles.

The invention also provides a kit comprising a container, wherein the container contains at least one reagent for specifically detecting the expression of at least one biomarker of the invention. In certain embodiments, the reagent is an antibody or nucleic acid probe that binds a biomarker of the invention.

In some embodiments, a kit comprises polynucleotides selected from the group consisting of SEQ ID NOs:62-79. In some embodiments, a kit comprises (a) a forward primer of SEQ ID NO:62, a reverse primer of SEQ ID NO:63, and a probe comprising SEQ ID NO:64; (b) a forward primer of SEQ ID NO:65, a reverse primer of SEQ ID NO:66, and a probe comprising SEQ ID NO:67; (c) a forward primer of SEQ ID NO:68, a reverse primer of SEQ ID NO:69, and a probe comprising SEQ ID NO:70; (d) a forward primer of SEQ ID NO:71, a reverse primer of SEQ ID NO:72, and a probe comprising SEQ ID NO:73; (e) a forward primer of SEQ ID NO:74, a reverse primer of SEQ ID NO:75, and a probe comprising SEQ ID NO:76; and (f) a forward primer of SEQ ID NO:77, a reverse primer of SEQ ID NO:78, and a probe comprising SEQ ID NO:79.

Where aspects or embodiments of the invention are described in terms of a Markush group or other grouping of alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTIONS OF THE DRAWINGS

FIGS. 1A-1H. Classification of responsive or non-responsive breast tumors. FIG. 1A. Breast tumor OMP-B34 cells were injected subcutaneously into NOD/SCID mice. FIG. 1B. Breast tumor OMP-B39 cells were injected subcutaneously into NOD/SCID mice. FIG. 1C. Breast tumor OMP-B44 cells were injected subcutaneously into NOD/SCID mice. FIG. 1D. Breast tumor OMP-B59 cells were injected subcutaneously into NOD/SCID mice. FIG. 1E. Breast tumor OMP-B60 cells were injected subcutaneously into NOD/SCID mice. FIG. 1F. Breast tumor UM-T01 cells were injected subcutaneously into NOD/SCID mice. FIG. 1G. Breast tumor UM-T03 cells were injected subcutaneously into NOD/SCID mice. FIG. 1H. Breast tumor UM-PE13 cells were injected subcutaneously into NOD/SCID mice. For each experiment, mice were treated with OMP-18R5 antibody (-▪-), taxol (-▴-), a combination of OMP-18R5 and taxol (-▾-), or a control antibody (--). Data is shown as tumor volume (mm³) over days post-treatment.

FIG. 2. Performance curve for the top 20 ranked genes.

FIG. 3. PCA plot of 6 selected genes.

FIG. 4. Correlation of the 6-gene biomarker signature with ratio of tumor volume.

FIG. 5. Prediction of tumor responsiveness based upon classification probability analysis. T=tumor used in training set for establishment of 6-gene signature.

FIGS. 6A-6F. In vivo validation of predictive biomarkers. FIG. 6A. Breast tumor OMP-B29 cells were injected subcutaneously into NOD/SCID mice. FIG. 6B. Breast tumor OMP-B71 cells were injected subcutaneously into NOD/SCID mice. FIG. 6C. Breast tumor OMP-B84 cells were injected subcutaneously into NOD/SCID mice. FIG. 6D. Breast tumor OMP-B90 cells were injected subcutaneously into NOD/SCID mice. FIG. 6E. Breast tumor UM-T02 cells were injected subcutaneously into NOD/SCID mice. FIG. 6F. Breast tumor UM-T06 cells were injected subcutaneously into NOD/SCID mice. For each experiment, mice were treated with OMP-18R5 antibody (-▪-), taxol (-▴-), a combination of OMP-18R5 and taxol (-▾-), or a control antibody (--). Data is shown as tumor volume (mm³) over days post-treatment.

FIG. 7. Population prevalence estimation of the 6-gene biomarker signature using three public datasets.

### DETAILED DESCRIPTION OF THE INVENTION

### I. Definitions

To facilitate an understanding of the present invention, a number of terms and phrases are defined below.

The term “biomarker” as used herein may include but is not limited to, nucleic acids and proteins, and variants and fragments thereof. A biomarker may include DNA comprising the entire or partial nucleic acid sequence encoding the biomarker, or the complement of such a sequence. Biomarker nucleic acids useful in the invention are considered to include both DNA and RNA comprising the entire or partial sequence of any of the nucleic acid sequences of interest. Biomarker proteins are considered to comprise the entire or partial amino acid sequence of any of the biomarker proteins or polypeptides.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen-binding site within the variable region of the immunoglobulin molecule. As used herein, the term encompasses intact polyclonal antibodies, intact monoclonal antibodies, single chain antibodies, antibody fragments (such as Fab, Fab′, F(ab′)2, and Fv fragments), single chain Fv (scFv) antibodies, multispecific antibodies such as bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises at least one antigen-binding site or epitope-binding site.

The term “variable region” of an antibody refers to the variable region of an antibody light chain, or the variable region of an antibody heavy chain, either alone or in combination. The variable region of a heavy chain or a light chain generally consists of four framework regions (FR) connected by three complementarity determining regions (CDRs), also known as “hypervariable regions”. The CDRs in each chain are held together in close proximity by the framework regions and contribute to the formation of the antigen-binding site(s) of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda, Md.), and (2) an approach based on crystallographic studies of antigen-antibody complexes (Al-Lazikani et al., 1997, J. Mol. Biol., 273:927-948). In addition, combinations of these two approaches are sometimes used in the art to determine CDRs.

The term “monoclonal antibody” as used herein refers to a homogeneous antibody population involved in the highly specific recognition and binding of a single antigenic determinant or epitope. This is in contrast to polyclonal antibodies that typically include a mixture of different antibodies directed against a variety of different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody portion, and any other modified immunoglobulin molecule comprising an antigen-binding site. Furthermore, “monoclonal antibody” refers to such antibodies made by any number of techniques, including but not limited to, hybridoma production, phage selection, recombinant expression, and transgenic animals.

The term “humanized antibody” as used herein refers to antibodies that are specific immunoglobulin chains, chimeric immunoglobulins, or fragments thereof that contain minimal non-human sequences. Methods used to generate humanized antibodies are well known in the art.

The term “human antibody” as used herein refers to an antibody produced by a human or an antibody having an amino acid sequence corresponding to an antibody produced by a human. A human antibody may be made using any of the techniques known in the art.

The term “chimeric antibody” as used herein refers to an antibody wherein the amino acid sequence of the immunoglobulin molecule is derived from two or more species. Typically, the variable regions of the light chain and the heavy chain correspond to the variable regions of an antibody derived from one species of mammals (e.g., mouse, rat, rabbit, etc.) with the desired specificity, affinity, and/or binding capability, while the constant regions correspond to sequences from an antibody derived from another species (usually human).

The term “affinity-matured antibody” as used herein refers to an antibody with one or more alterations in one or more CDRs thereof that result in an improvement in the affinity of the antibody for antigen, compared to a parent antibody that does not possess those alterations(s). The definition also includes alterations in non-CDR residues made in conjunction with alterations to CDR residues. Preferred affinity-matured antibodies will have nanomolar or even picomolar affinities for the target antigen. Affinity-matured antibodies are produced by procedures known in the art. For example, techniques may include affinity maturation by VH and VL domain shuffling, random mutagenesis of CDR and/or framework residues, and site-directed mutagenesis.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5 or 8-10 amino acids in a unique spatial conformation.

The terms “selectively binds” or “specifically binds” mean that a binding agent or an antibody reacts or associates more frequently, more rapidly, with greater duration, with greater affinity, or with some combination of the above to the epitope, protein, or target molecule than with alternative substances, including unrelated or related proteins. In certain embodiments “specifically binds” means, for instance, that an antibody binds a protein with a K_{D} of about 0.1 mM or less, but more usually less than about 1 μM. In certain embodiments, “specifically binds” means that an antibody binds a target at times with a K_{D} of at least about 0.1 μM or less, at other times at least about 0.01 μM or less, and at other times at least about 1 nM or less. Because of the sequence identity between homologous proteins in different species, specific binding can include an antibody that recognizes a protein in more than one species (e.g., human FZD and mouse FZD). Likewise, because of homology within certain regions of polypeptide sequences of different proteins, specific binding can include an antibody (or other polypeptide or binding agent) that recognizes more than one protein (e.g., human FZD 1 and human FZD7). It is understood that, in certain embodiments, an antibody or binding agent that specifically binds a first target may or may not specifically bind a second target. As such, “specific binding” does not necessarily require (although it can include) exclusive binding, i.e. binding to a single target. Thus, a binding agent may, in certain embodiments, specifically bind more than one target. In certain embodiments, multiple targets may be bound by the same binding site on the agent or antibody. For example, an antibody may, in certain instances, comprise two identical antigen-binding sites, each of which specifically binds the same epitope on two or more proteins. In certain alternative embodiments, an antibody may be bispecific or multispecific and comprise at least two antigen-binding sites with differing specificities. By way of non-limiting example, a bispecific agent may comprise one binding site that recognizes a target on one protein (e.g., human FZD) and further comprise a second, different binding site that recognizes a different target on a second protein (e.g., a human WNT protein). Generally, but not necessarily, reference to binding means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies, in certain embodiments, the polypeptides can occur as single chains or associated chains (e.g., dimers).

The terms “polynucleotide” and “nucleic acid” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

“Conditions of high stringency” may be identified by conditions that: (1) employ low ionic strength and high temperature for washing, for example 15 mM sodium chloride/1.5 mM sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 in 5× SSC (0.75M NaCl, 75mM sodium citrate) at 42° C.; or (3) employ during hybridization 50% formamide in 5× SSC, 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2× SSC and 50% formamide, followed by a wash consisting of 0.1× SSC containing EDTA at 55° C.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variations thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues, at least about 60-80 residues in length or any integral value therebetween. In some embodiments, identity exists over a longer region than 60-80 residues, such as at least about 80-100 residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Preferably, conservative substitutions in the sequences of the polypeptides and antibodies of the invention do not abrogate the binding of the polypeptide or antibody containing the amino acid sequence, to the antigen to which the polypeptide or antibody binds. Methods of identifying nucleotide and amino acid conservative substitutions which do not eliminate antigen binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

As used herein the term “soluble receptor” refers to an extracellular domain (or a fragment thereof) of a receptor protein preceding the first transmembrane domain of the receptor that can be secreted from a cell in soluble form. Generally this is the N-terminal portion of the receptor protein.

As used herein the term “FZD soluble receptor” or “soluble FZD receptor” refers to an N-terminal extracellular fragment of a FZD receptor protein preceding the first transmembrane domain of the receptor that can be secreted from a cell in soluble form. FZD soluble receptors comprising the entire N-terminal extracellular domain (ECD) as well as smaller fragments are encompassed by the term. Thus, FZD soluble receptors comprising a FZD Fri domain are also included in this term.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (non-cancerous) or malignant (cancerous) including pre-cancerous lesions.

The term “metastasis” as used herein refers to the process by which a cancer spreads or transfers from the site of origin to other regions of the body with the development of a similar cancerous lesion at a new location. A “metastatic” or “metastasizing” cell is one that loses adhesive contacts with neighboring cells and migrates (e.g., via the bloodstream or lymph) from the primary site of disease to secondary sites.

The terms “cancer stem cell” and “CSC” and “tumor stem cell” and “tumor initiating cell” are used interchangeably herein and refer to cells from a cancer or tumor that: (1) have extensive proliferative capacity; 2) are capable of asymmetric cell division to generate one or more types of differentiated cell progeny wherein the differentiated cells have reduced and/or limited proliferative or developmental potential; and (3) are capable of symmetric cell divisions for self-renewal or self-maintenance. These properties confer on the cancer stem cells the ability to form or establish a tumor or cancer upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a random sample of cells from the tumor to form palpable tumors upon serial transplantation into immunocompromised hosts (e.g., mice). This definition also includes enriched and/or isolated populations of cancer stem cells that form palpable tumors upon serial transplantation into immunocompromised hosts (e.g., mice).

The term “patient” refers to any animal (e.g., a mammal), including, but not limited to, humans, non-human primates, canines, felines, rodents, and the like, which is to be the recipient of a particular treatment. Typically, the terms “patient” and “subject” are used interchangeably herein in reference to a human patient.

The term “pharmaceutically acceptable” refers to a product or compound approved (or approvable) by a regulatory agency of the Federal government or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier, or adjuvant that can be administered to a subject, together with at least one agent (e.g., an antibody) of the present disclosure, and which does not destroy the activity of the agent. The excipient, carrier, or adjuvant should be non-toxic when administered with an agent in doses sufficient to deliver a therapeutic effect.

The terms “effective amount” or “therapeutically effective amount” or “therapeutic effect” refer to an amount of a binding agent, an antibody, polypeptide, polynucleotide, small organic molecule, or other drug effective to “treat” a disease or disorder in a subject or mammal In the case of cancer, the therapeutically effective amount of a drug (e.g., an antibody) has a therapeutic effect and as such can reduce the number of cancer cells; decrease tumorigenicity, tumorigenic frequency, or tumorigenic capacity; reduce the number or frequency of cancer stem cells; reduce the tumor size; reduce the cancer cell population; inhibit and/or stop cancer cell infiltration into peripheral organs including, for example, the spread of cancer into soft tissue and bone; inhibit and/or stop tumor or cancer cell metastasis; inhibit and/or stop tumor or cancer cell growth; relieve to some extent one or more of the symptoms associated with the cancer; reduce morbidity and mortality; improve quality of life; or a combination of such effects. To the extent the agent, for example an antibody, prevents growth and/or kills existing cancer cells, it can be referred to as cytostatic and/or cytotoxic.

The terms “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus those in need of treatment include those already diagnosed with the disorder; those prone to have the disorder; and those in whom the disorder is to be prevented. In some embodiments, a subject is successfully “treated” according to the methods of the present invention if the patient shows one or more of the following: a reduction in the number of and/or complete absence of cancer cells; a reduction in the tumor size; an inhibition of tumor growth; inhibition of and/or an absence of cancer cell infiltration into peripheral organs including the spread of cancer cells into soft tissue and bone; inhibition of and/or an absence of tumor or cancer cell metastasis; inhibition and/or an absence of cancer growth; relief of one or more symptoms associated with the specific cancer; reduced morbidity and mortality; improvement in quality of life; reduction in tumorigenicity; reduction in the number or frequency of cancer stem cells; or some combination of such effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of and/or “consisting essentially of are also provided. It is also understood that wherever embodiments are described herein with the language “consisting essentially of otherwise analogous embodiments described in terms of “consisting of are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Methods of use of Predictive Biomarkers

Provided herein are methods for identifying, classifying, and/or selecting tumors and/or patients with cancer that are likely to be responsive (“sensitive”) or non-responsive (“resistant”) to treatment with a Wnt pathway inhibitor. In addition, provided are methods for treating patients with cancer who are likely to respond to treatment, are predicted to respond to treatment, and/or have been identified to respond to treatment with a Wnt pathway inhibitor.

Provided herein is a method of identifying a human tumor that is likely to be responsive or non-responsive to treatment with a Wnt pathway inhibitor, the method comprising: (a) obtaining a sample of the human tumor; (b) measuring the expression level of each biomarker of a biomarker signature in the sample, wherein the biomarker signature comprises one or more of the biomarkers FBXW2, CCND2, RHOU, CRBP2, WIF1, and DKK1; and (c) identifying the tumor as likely to be responsive or non-responsive to treatment based upon the expression level of the biomarkers. In some embodiments, a method of identifying a human tumor that is likely to be responsive or non-responsive to treatment with a Wnt pathway inhibitor comprises: (a) obtaining a sample of the human tumor; (b) measuring the expression level of each biomarker of a biomarker signature in the sample, wherein the biomarker signature comprises one or more of the biomarkers FBXW2, CCND2, RHOU, CRBP2, WIF1, and DKK1; and (c) calculating a decision value based upon the standardized expression of the biomarkers in the biomarker signature; wherein a positive decision value indicates the tumor is predicted to be responsive to the Wnt pathway inhibitor and a negative decision value indicates the tumor is predicted to be non-responsive to the Wnt pathway inhibitor.

Provided herein is a method of classifying a human tumor as likely to be responsive or non-responsive to treatment with a Wnt pathway inhibitor, the method comprising: (a) obtaining a sample of the human tumor; (b) measuring the expression level of each biomarker of a biomarker signature in the sample, wherein the biomarker signature comprises one or more of the biomarkers FBXW2, CCND2, RHOU, CTBP2, WIF1, and DKK1; and (c) classifying the tumor as likely to be responsive or non-responsive to treatment based upon the expression level of the biomarkers. In some embodiments, a method of classifying a human tumor as likely to be responsive or non-responsive to treatment with a Wnt pathway inhibitor comprises: (a) obtaining a sample of the human tumor; (b) measuring the expression level of each biomarker of a biomarker signature in the sample, wherein the biomarker signature comprises one or more of the biomarkers FBXW2, CCND2, RHOU, CTBP2, WIF1, and DKK1; and (c) calculating a decision value based upon the standardized expression of the biomarkers in the biomarker signature; wherein a positive decision value indicates the tumor is predicted to be responsive to the Wnt pathway inhibitor and a negative decision value indicates the tumor is predicted to be non-responsive to the Wnt pathway inhibitor.

Provided herein is a method of determining the responsiveness (or sensitivity) of a human tumor to treatment with a Wnt pathway inhibitor, the method comprising: (a) obtaining a sample of the human tumor; (b) measuring the expression level of each biomarker of a biomarker signature in the sample, wherein the biomarker signature comprises one or more of the genes FBXW2, CCND2, RHOU, CTBP2, WIF 1, and DKK1; and (c) determining the responsiveness of the tumor to treatment based upon the expression level of the biomarkers. In some embodiments, a method of determining the responsiveness or sensitivity of a human tumor to treatment with a Wnt pathway inhibitor comprises: (a) obtaining a sample of the human tumor; (b) measuring the expression level of each biomarker of a biomarker signature in the sample, wherein the biomarker signature comprises one or more of the genes FBXW2, CCND2, RHOU, CTBP2, WIF1, and DKK1; and (c) calculating a decision value based upon the standardized expression of the biomarkers in the biomarker signature; wherein a positive decision value indicates the tumor is predicted to be responsive to the Wnt pathway inhibitor.

Provided herein is a method of identifying a patient with cancer who is likely to respond to treatment with a Wnt pathway inhibitor, the method comprising: (a) obtaining a sample of the patient's tumor; (b) measuring the expression level of each biomarker of a biomarker signature in the sample, wherein the biomarker signature comprises one or more of the biomarkers FBXW2, CCND2, RHOU, CTBP2, WIF 1, and DKK1; and (c) identifying the patient who is likely to respond to treatment based upon the expression level of the biomarkers. In some embodiments, a method of identifying a patient with cancer who is likely to respond to treatment with a Wnt pathway inhibitor comprises: (a) obtaining a sample of the patient's tumor; (b) measuring the expression level of each biomarker of a biomarker signature in the sample, wherein the biomarker signature comprises one or more of the biomarkers FBXW2, CCND2, RHOU, CTBP2, WIF1, and DKK1; and (c) calculating a decision value based upon the standardized expression of the biomarkers in the biomarker signature; wherein a positive decision value indicates that the patient is predicted to respond to treatment with the Wnt pathway inhibitor. In some embodiments, the method further comprises selecting the patient for treatment when their tumor sample has a positive decision value. In some embodiments, the method further comprises administering a therapeutically effective amount of the Wnt pathway inhibitor to the patient.

Provided herein is a method of selecting a patient with cancer for treatment with a Wnt pathway inhibitor, the method comprising: (a) obtaining a sample of the patient's tumor; (b) measuring the expression level of each biomarker of a biomarker signature in the sample, wherein the biomarker signature comprises one or more of the biomarkers FBXW2, CCND2, RHOU, CTBP2, WIF1, and DKK1; (c) selecting the patient for treatment based upon the expression level of the biomarkers. In some embodiments, a method of selecting a patient with cancer for treatment with a Wnt pathway inhibitor comprises: (a) obtaining a sample of the patient's tumor; (b) measuring the expression level of each biomarker of a biomarker signature in the sample, wherein the biomarker signature comprises one or more of the biomarkers FBXW2, CCND2, RHOU, CTBP2, WIF1, and DKK1; (c) calculating a decision value based upon the standardized expression of the biomarkers in the biomarker signature; and (d) selecting the patient for treatment when their tumor sample has a positive decision value. In some embodiments, the method further comprises administering a therapeutically effective amount of the Wnt pathway inhibitor to the patient.

Provided herein is a method of treating cancer in a patient, comprising: (a) identifying if the patient is likely to respond to treatment with a Wnt pathway inhibitor, wherein the identification comprises: (i) obtaining a sample of the patient's cancer; (ii) measuring the expression level of each biomarker of a biomarker signature in the sample, wherein the biomarker signature comprises one or more of the biomarkers FBXW2, CCND2, RHOU, CTBP2, WIF1, and DKK1; and (iii) identifying the patient who is likely to respond to treatment based upon the expression level of the biomarkers; and (b) administering to the patient who is likely to response to treatment an effective amount of the Wnt pathway inhibitor. In some embodiments, a method of treating cancer in a patient comprises: (a) identifying if the patient is likely to respond to treatment with a Wnt pathway inhibitor, wherein the identification comprises: (i) obtaining a sample of the patient's cancer; (ii) measuring the expression level of each biomarker of a biomarker signature in the sample, wherein the biomarker signature comprises one or more of the biomarkers FBXW2, CCND2, RHOU, CTBP2, WIF1, and DKK1; and (iii) calculating a decision value based upon the standardized expression of the biomarkers in the signature; wherein a positive decision value indicates that the patient is predicted to respond to treatment; and (b) administering to the patient who is predicted to response to treatment an effective amount of the Wnt pathway inhibitor.

In another aspect, the invention provides a method of treating cancer in a patient, comprising: administering an effective amount of a Wnt pathway inhibitor to the patient; wherein the patient is predicted to respond to treatment with a Wnt inhibitor based upon expression levels of a biomarker signature in a patient tumor sample, wherein the signature comprises one or more of the biomarkers FBXW2, CCND2, RHOU, CTBP2, WIF1, and DKK1. In some embodiments, a method of treating cancer in a patient comprises: administering an effective amount of a Wnt pathway inhibitor to the patient; wherein the patient is predicted to respond to treatment based upon a positive decision value calculated from the weighted sum of the standardized expression of biomarkers in a biomarker signature in a patient tumor sample, wherein the set of biomarkers comprises one or more of the biomarkers FBXW2, CCND2, RHOU, CTBP2, WIF1, and DKK1.

Provided herein is a method for increasing the likelihood of effective treatment with a Wnt pathway inhibitor, comprising: (a) identifying if a patient has a tumor that is likely to respond to treatment with a Wnt pathway inhibitor, wherein the identification comprises: (i) obtaining a sample of the patient's cancer; (ii) measuring the expression level of each biomarker of a biomarker signature in the sample, wherein the biomarker signature comprises one or more of the biomarkers FBXW2, CCND2, RHOU, CTBP2, WIF1, and DKK1; and (iii) identifying the patient who is likely to respond to treatment based upon the expression level of the biomarkers; and (b) administering an effective amount of the Wnt pathway inhibitor to the patient. In some embodiments, a method for increasing the likelihood of effective treatment with a Wnt pathway inhibitor comprises: (a) identifying if a patient has a tumor that is likely to respond to treatment with a Wnt pathway inhibitor, wherein the identification comprises: (i) obtaining a sample of the patient's cancer; (ii) measuring the expression level of each biomarker of a biomarker signature in the sample, wherein the biomarker signature comprises one or more of the biomarkers FBXW2, CCND2, RHOU, CTBP2, WIF1, and DKK1; and (iii) calculating a decision value based upon the standardized expression of the biomarkers in the biomarker signature; wherein a positive decision value indicates that the patient is predicted to respond to treatment; and (b) administering an effective amount of the Wnt pathway inhibitor to the patient whose tumor has a positive decision value.

In another aspect, the invention provides a method for increasing the likelihood of effective treatment with a Wnt pathway inhibitor, comprising: administering an effective amount of a Wnt pathway inhibitor to a patient; wherein the patient is identified as likely to respond to treatment with a Wnt inhibitor based upon expression levels of a biomarker signature in a patient tumor sample, wherein the signature comprises one or more of the biomarkers FBXW2, CCND2, RHOU, CTBP2, WIF1, and DKK1. In some embodiments, a method for increasing the likelihood of effective treatment with a Wnt pathway inhibitor comprises: administering an effective amount of a Wnt pathway inhibitor to a patient; wherein the patient is identified as likely to respond to treatment based upon a positive decision value calculated from the weighted sum of the standardized expression of biomarkers in a biomarker signature in a patient tumor sample, wherein the set of biomarkers comprises one or more of the biomarkers FBXW2, CCND2, RHOU, CTBP2, WIF1, and DKK1. In some embodiments, the patient is identified as likely to respond to treatment with a Wnt pathway inhibitor in combination with paclitaxel. In some embodiments, the method comprises administering to the patient the Wnt pathway inhibitor in combination with paclitaxel.

Provided herein is a use for identifying a human tumor that is likely to be responsive or non-responsive to treatment with a Wnt pathway inhibitor, wherein the use comprises (a) obtaining a sample of the human tumor; (b) measuring the expression level of each biomarker of a biomarker signature in the sample, wherein the biomarker signature comprises one or more of the biomarkers FBXW2, CCND2, RHOU, CRBP2, WIF1, and DKK1; and (c) calculating a decision value based upon the standardized expression of the biomarkers in the biomarker signature; wherein a positive decision value indicates the tumor is predicted to be responsive to the Wnt pathway inhibitor and a negative decision value indicates the tumor is predicted to be non-responsive to the Wnt pathway inhibitor.

Provided herein is a use for classifying a human tumor as likely to be responsive or non-responsive to treatment with a Wnt pathway inhibitor, wherein the use comprises (a) obtaining a sample of the human tumor; (b) measuring the expression level of each biomarker of a biomarker signature in the sample, wherein the biomarker signature comprises one or more of the biomarkers FBXW2, CCND2, RHOU, CTBP2, WIF1, and DKK1; and (c) calculating a decision value based upon the standardized expression of the biomarkers in the biomarker signature; wherein a positive decision value indicates the tumor is predicted to be responsive to the Wnt pathway inhibitor and a negative decision value indicates the tumor is predicted to be non-responsive to the Wnt pathway inhibitor.

Provided herein is a use for determining the sensitivity of a human tumor to treatment with a Wnt pathway inhibitor, wherein the use comprises (a) obtaining a sample of the human tumor; (b) measuring the expression level of each biomarker of a biomarker signature in the sample, wherein the biomarker signature comprises one or more of the genes FBXW2, CCND2, RHOU, CTBP2, WIF1, and DKK1; and (c) calculating a decision value based upon the standardized expression of the biomarkers in the biomarker signature; wherein a positive decision value indicates the tumor is predicted to be responsive to the Wnt pathway inhibitor.

Provided herein is a use for identifying a patient with cancer who is likely to respond to treatment with a Wnt pathway inhibitor, wherein the use comprises (a) obtaining a sample of the patient's tumor; (b) measuring the expression level of each biomarker of a biomarker signature in the sample, wherein the biomarker signature comprises one or more of the biomarkers FBXW2, CCND2, RHOU, CTBP2, WIF1, and DKK1; and (c) calculating a decision value based upon the standardized expression of the biomarkers in the biomarker signature; wherein a positive decision value indicates that the patient is predicted to respond to treatment with the Wnt pathway inhibitor. In some embodiments, the use further comprises selecting the patient for treatment when their tumor sample has a positive decision value. In some embodiments, the use further comprises administering a therapeutically effective amount of the Wnt pathway inhibitor to the patient.

Provided herein is a use for selecting a patient with cancer for treatment with a Wnt pathway inhibitor, wherein the use comprises (a) obtaining a sample of the patient's tumor; (b) measuring the expression level of each biomarker of a biomarker signature in the sample, wherein the biomarker signature comprises one or more of the biomarkers FBXW2, CCND2, RHOU, CTBP2, WIF1, and DKK1; (c) calculating a decision value based upon the standardized expression of the biomarkers in the biomarker signature; and (d) selecting the patient for treatment when their tumor sample has a positive decision value. In some embodiments, the use further comprises administering a therapeutically effective amount of the Wnt pathway inhibitor to the patient.

Provided herein is a Wnt pathway inhibitor for use in treating cancer in a patient, the use comprising: (a) identifying if the patient is likely to respond to treatment with a Wnt pathway inhibitor, wherein the identification comprises: (i) obtaining a sample of the patient's cancer; (ii) measuring the expression level of each biomarker of a biomarker signature in the sample, wherein the biomarker signature comprises one or more of the biomarkers FBXW2, CCND2, RHOU, CTBP2, WIF1, and DKK1; and (iii) calculating a decision value based upon the standardized expression of the biomarkers in the signature; wherein a positive decision value indicates that the patient is predicted to respond to treatment; and (b) administering to the patient who is predicted to response to treatment an effective amount of the Wnt pathway inhibitor.

Provided herein is a use for increasing the likelihood of effective treatment with a Wnt pathway inhibitor, the use comprising: (a) identifying if a patient has a tumor that is likely to respond to treatment with a Wnt pathway inhibitor, wherein the identification comprises: (i) obtaining a sample of the patient's cancer; (ii) measuring the expression level of each biomarker of a biomarker signature in the sample, wherein the biomarker signature comprises one or more of the biomarkers FBXW2, CCND2, RHOU, CTBP2, WIF1, and DKK1; and (iii) calculating a decision value based upon the standardized expression of the biomarkers in the biomarker signature; wherein a positive decision value indicates that the patient is predicted to respond to treatment; and (b) administering an effective amount of the Wnt pathway inhibitor to the patient whose tumor has a positive decision value.

Provided herein is a Wnt pathway inhibitor for use in treating cancer in a patient identified to likely to respond to treatment with a Wnt pathway inhibitor wherein the identification of the patient comprises: (i) measuring the expression level of each biomarker of a biomarker signature in the cancer sample obtained from the patient, wherein the biomarker signature comprises one or more of the biomarkers FBXW2, CCND2, RHOU, CTBP2, WIF1, and DKK1; and (ii) calculating a decision value based upon the standardized expression of the biomarkers in the signature; wherein a positive decision value indicates that the patient is predicted to respond to treatment.

Provided herein is a Wnt pathway inhibitor for use in treating cancer in a patient, wherein the patient is one for whom a positive decision value is calculated based upon the standardized expression of each biomarker of the biomarker signature in a cancer sample of the patient, wherein the biomarker signature comprises one or more of the biomarkers FBXW2, CCND2, RHOU, CTBP2, WIF1, and DKK1.

In some embodiments of the methods described herein, the biomarker signature comprises two or more of the biomarkers FBXW2, CCND2, RHOU, CTBP2, WIF1, and DKK1. In some embodiments, the biomarker signature comprises three or more of the biomarkers FBXW2, CCND2, RHOU, CTBP2, WIF1, and DKK1. In some embodiments, the biomarker signature comprises four or more of the biomarkers FBXW2, CCND2, RHOU, CTBP2, WIF1, and DKK1. In some embodiments, the biomarker signature comprises five or more of the biomarkers FBXW2, CCND2, RHOU, CTBP2, WIF1, and DKK1. In some embodiments, the biomarker signature comprises FBXW2, CCND2, RHOU, CTBP2, WIF1, and DKK1. In some embodiments, the biomarker signature consists of FBXW2, CCND2, RHOU, CTBP2, WIF1, and DKK1.

In some embodiments, the biomarker signature comprises one or more additional biomarkers, in addition to at least one of the biomarkers FBXW2, CCND2, RHOU, CTBP2, WIF1, and DKK1. In some embodiments, the biomarker signature comprises one or more additional biomarkers selected from the genes listed in Table 2, in addition to at least one of the biomarkers FBXW2, CCND2, RHOU, CTBP2, WIF1, and DKK1. In some embodiments, the biomarker signature comprises one or more of the biomarkers EP300, CTBP1, WNT6, WNT9A, SNT3, FZD2, FZD7, APC, TLE2, DVL2, PITX2, WISP1, GSK3B, and LEF1, in addition to at least one of the biomarkers FBXW2, CCND2, RHOU, CTBP2, WIF1, and DKK1. In some embodiments, the biomarker signature comprises one or more of the biomarkers FBXW2, CCND2, RHOU, CTBP2, WIF1, DKK1, EP300, and CTBP1. In some embodiments, the biomarker signature comprises one or more of the biomarkers FBXW2, CCND2, RHOU, CTBP2, WIF1, DKK1, EP300, CTBP1, WNT6, WNT3, FZD2, APC, TLE2, DVL2, PITX2, WISP1, GSK3B, WNT9A, FZD7, and LEF1.

In some embodiments of the methods described herein, the biomarker signature comprises FBXW2. In some embodiments, the biomarker signature comprises CCND2. In some embodiments, the biomarker signature comprises RHOU. In some embodiments, the biomarker signature comprises CTBP2. In some embodiments, the biomarker signature comprises WIF1. In some embodiments, the biomarker signature comprises DKK1.

In some embodiments of the methods described herein, the biomarker signature comprises FBXW2 and CCND2. In some embodiments, the biomarker signature comprises FBXW2 and RHOU. In some embodiments, the biomarker signature comprises FBXW2 and CTBP2. In some embodiments, the biomarker signature comprises FBXW2 and WIF1. In some embodiments, the biomarker signature comprises FBXW2 and DKK1. In some embodiments, the biomarker signature comprises CCND2 and RHOU. In some embodiments, the biomarker signature comprises CCND2 and CTBP2. In some embodiments, the biomarker signature comprises CCND2 and WIF1. In some embodiments, the biomarker signature comprises CCND2 and DKK1. In some embodiments, the biomarker signature comprises RHOU and CTBP2. In some embodiments, the biomarker signature comprises RHOU and WIF1. In some embodiments, the biomarker signature comprises RHOU and DKK1. In some embodiments, the biomarker signature comprises CTBP2 and WIF1. In some embodiments, the biomarker signature comprises CTBP2 and DKK1. In some embodiments, the biomarker signature comprises WIF1 and DKK1.

In some embodiments of the methods described herein, the biomarker signature comprises FBXW2, CCND2, and RHOU. In some embodiments, the biomarker signature comprises FBXW2, CCND2, and CTBP2. In some embodiments, the biomarker signature comprises FBXW2, CCND2, and WIF1. In some embodiments, the biomarker signature comprises FBXW2, CCND2, and DKK1. In some embodiments, the biomarker signature comprises FBXW2, RHOU, and CTBP2. In some embodiments, the biomarker signature comprises FBXW2, RHOU, and WIF1. In some embodiments, the biomarker signature comprises FBXW2, RHOU, and DKK1. In some embodiments, the biomarker signature comprises FBXW2, CTBP2, and WIF1. In some embodiments, the biomarker signature comprises FBXW2, CTBP2, and DKK1. In some embodiments, the biomarker signature comprises FBXW2, WIF1, and DKK1. In some embodiments, the biomarker signature comprises CCND2, RHOU, and CTBP2. In some embodiments, the biomarker signature comprises CCND2, RHOU, and WIF1. In some embodiments, the biomarker signature comprises CCND2, RHOU, and DKK1. In some embodiments, the biomarker signature comprises CCND2, CTBP2, and WIF1. In some embodiments, the biomarker signature comprises CCND2, CTBP2, and DKK1. In some embodiments, the biomarker signature comprises CCND2, WIF1, and DKK1. In some embodiments, the biomarker signature comprises RHOU, CTBP2, and WIF1. In some embodiments, the biomarker signature comprises RHOU, CTBP2, and DKK1. In some embodiments, the biomarker signature comprises RHOU, WIF1, and DKK1. In some embodiments, the biomarker signature comprises CTBP2, WIF1, and DKK1.

In some embodiments of the methods described herein, the biomarker signature comprises FBXW2, CCND2, RHOU, and CTBP2. In some embodiments, the biomarker signature comprises FBXW2, CCND2, RHOU, and WIF1. In some embodiments, the biomarker signature comprises FBXW2, CCND2, RHOU, and DKK1. In some embodiments, the biomarker signature comprises FBXW2, RHOU, CTBP2, and WIF1. In some embodiments, the biomarker signature comprises FBXW2, RHOU, CTBP2, and DKK1. In some embodiments, the biomarker signature comprises FBXW2, CTBP2, WIF1, and DKK1. In some embodiments, the biomarker signature comprises CCND2, RHOU, CTBP2, and WIF1. In some embodiments, the biomarker signature comprises CCND2, RHOU, CTBP2, and DKK1. In some embodiments, the biomarker signature comprises CCND2, CTBP2, WIF1, and DKK1. In some embodiments, the biomarker signature comprises RHOU, CTBP2, WIF1, and DKK1. In some embodiments, any of these signatures may comprise one or more additional biomarkers.

In some embodiments of the methods described herein, the biomarker signature comprises FBXW2, CCND2, RHOU, CTBP2, and WIF1. In some embodiments, the biomarker signature comprises FBXW2, CCND2, RHOU, CTBP2, and DKK1. In some embodiments, the biomarker signature comprises FBXW2, CCND2, CTBP2, WIF1, and DKK1. In some embodiments, the biomarker signature comprises FBXW2, CCND2, RHOU, WIF1, and DKK1. In some embodiments, the biomarker signature comprises FBXW2, RHOU, CTBP2, WIF1, and DKK1. In some embodiments, the biomarker signature comprises CCND2, RHOU, CTBP2, WIF1, and DKK1.

In some embodiments, the sample includes, but is not limited to, any clinically relevant tissue sample, such as a tumor biopsy, a core biopsy tissue sample, a fine needle aspirate, a hair follicle, or a sample of bodily fluid, such as blood, plasma, serum, lymph, ascitic fluid, cystic fluid, or urine. In some embodiments, the sample is taken from a patient having a tumor or cancer. In some embodiments, the sample is a primary tumor. In some embodiments, the sample is a metastasis. The sample may be taken from a human, or from non-human mammals such as, mice, rats, non-human primates, canines, felines, ruminants, swine, or sheep. In some embodiments, samples are taken from a subject at multiple time points, for example, before treatment, during treatment, and/or after treatment. In some embodiments, samples are taken from different locations in the subject, for example, a sample from a primary tumor and a sample from a metastasis in a distant location.

In some embodiments, the sample is a paraffin-embedded fixed tissue sample. In some embodiments, the sample is a formalin-fixed paraffin embedded (FFPE) tissue sample. In some embodiments, the sample is a fresh tissue (e.g., tumor) sample. In some embodiments, the sample is a frozen tissue sample. In some embodiments, the sample is a fresh frozen (FF) tissue (e.g., tumor) sample. In some embodiments, the sample is a cell isolated from a fluid. In some embodiments, the sample comprises circulating tumor cells (CTCs). In some embodiments, the sample is an archival tissue sample. In some embodiments, the sample is an archival tissue sample with known diagnosis, treatment, and/or outcome history. In some embodiments, the sample is a block of tissue. In some embodiments, the sample is dispersed cells. In some embodiments, the sample size is from about 1 cell to about 1×10⁶ cells or more. In some embodiments, the sample size is about 10 cells to about 1×10⁵ cells. In some embodiments, the sample size is about 10 cells to about 10,000 cells. In some embodiments, the sample size is about 10 cells to about 1,000 cells. In some embodiments, the sample size is about 10 cells to about 100 cells. In some embodiments, the sample size is about 1 cell to about 10 cells. In some embodiments, the sample size is a single cell.

In some embodiments, the sample is processed to DNA or RNA. In some embodiments, RNA is isolated from the sample. In some embodiments, mRNA is isolated from the sample. In some embodiments, RNA is isolated from cells by procedures that involve cell lysis and denaturation of the proteins contained therein. In some embodiments, DNase is added to remove DNA. In some embodiments, RNase inhibitors are added to the lysis buffer. In some embodiments, a protein denaturation/digestion step is added to the protocol. Methods for preparing total and mRNA are well known in the art and RNA isolation kits are commercially available (e.g., RNeasy mini kit, Qiagen, USA). In some embodiments, the RNA is amplified by PCR-based techniques.

Determination of biomarker expression levels may be performed by any suitable method including, but are not limited to, methods based on analyses of polynucleotide expression, sequencing of polynucleotides, and/or analyses of protein expression. For example, determination of biomarker expression levels may be performed by detecting the expression of mRNA expressed from the genes of interest, and/or by detecting the expression of a polypeptide encoded by the genes.

Commonly used methods for the analysis of polynucleotides, include Southern blot analysis, Northern blot analysis, and in situ hybridization, RNAse protection assays, and polymerase chain reaction (PCR)-based methods, such as reverse transcription polymerase chain reaction (RT-PCR), quantitative PCR (qPCR) as known as real-time PCR, TaqMan™, TaqMan™ low density array (TLDA), anchored PCR, competitive PCR, rapid amplification of cDNA ends (RACE), and microarray analyses. RT-PCR is a quantitative method that can be used to compare mRNA levels in different samples to examine gene expression profiles. A variation of RT-PCR is real time quantitative PCR, which measures PCR product accumulation through a dual-labeled fluorigenic probe (e.g., TaqMan™ probe). There are many other PCR-based techniques known to one of skill in the art, including but not limited to, differential display, amplified fragment length polymorphism, BeadArray™ technology, high coverage expression profiling (HiCEP) and digital PCR. Representative methods for sequencing-based gene expression analyses include Serial Analysis of Gene Expression (SAGE), Massively Parallel Signature Sequencing (MPSS), and NexGen sequencing analysis, including mRNA sequencing.

In certain embodiments, the biomarker expression is determined using a qPCR assay. For example, total RNA is extracted from a fresh frozen (FF) tissue sample or total RNA is extracted from a macro-dissected formalin-fixed paraffin embedded (FFPE) tissue sample. The quantity and quality of the total RNA is assessed by standard spectrophotometry and/or any other appropriate method (e.g., an Agilent Bioanalyzer). Following RNA extraction, the RNA sample is reverse transcribed using standard methods and/or a commercially available cDNA synthesis kit (e.g., Roche Transcriptor First Strand cDNA synthesis kit). The resultant cDNA is pre-amplified using, for example, an ABI pre-amplification kit. Expression of the biomarker(s) (e.g., FBXW2, CCND2, RHOU, CTBP2, WIF1, and/or DKK1) are assessed on, for example, a Roche Lightcycler 480 system (Roche Diagnostics) using an ABI TaqMan Gene Expression Mastermix. qPCR reactions are performed in triplicate. For each assay a subset of the samples is run without reverse transcription (the RT-neg control), as well as, control samples run without template. A universal human reference RNA sample is included on each plate to act as a positive control. Suitable reference genes are identified from a standard panel of reference genes. Candidate reference genes are selected with different cellular functions to eliminate risk of co-regulation. The most suitable reference genes are evaluated and selected using specific software and algorithms (e.g., Genex software; GeNorm and Normfinder algorithms). The expression level of each biomarker is normalized using the selected optimum reference genes. In some embodiments, these normalized (or standardized) expression values for each biomarker are used to calculate the decision value of the sample. In some embodiments, these normalized (or standardized) expression values for each biomarker are used to calculate an expression level.

In some embodiments, biomarker expression is determined using a PCR-based assay comprising specific primers and/or probes for each biomarker (e.g., FBXW2, CCND2, RHOU, CTBP2, WIF1, and/or DKK1). As used herein, the term “probe” refers to any molecule that is capable of selectively binding a specifically intended target biomolecule. Probes can be synthesized by one of skill in the art using known techniques, or derived from biological preparations. Probes may include but are not limited to, RNA, DNA, proteins, peptides, aptamers, antibodies, and organic molecules. The term “primer” or “probe” encompasses oligonucleotides that have a sequence of a specific SEQ ID NO or oligonucleotides that have a sequence complementary to a specific SEQ ID NO. In some embodiments, the probe is modified. In some embodiments, the probe is modified with a quencher. In some embodiments, the probe is labeled. Labels can include, but are not limited to, colorimetric, fluorescent, chemiluminescent, or bioluminescent labels.

In some embodiments, biomarker expression of each biomarker is determined using a specific primer set and probe. In some embodiments, a specific primer set consists of a forward primer and a reverse primer. In some embodiments, CCND2 expression is determined using a polynucleotide comprising the sequence of GCTGTCTCTGATCCGCAAGC (SEQ ID NO:62), a polynucleotide comprising the sequence of GACGGTGGGTACATGGCAAAC (SEQ ID NO:63), and a polynucleotide comprising the sequence of CCTTCATTGCTCTGTGTGCCACCGAC (SEQ ID NO:64), or complements thereof. In some embodiments, CCND2 expression is determined using a forward primer of sequence GCTGTCTCTGATCCGCAAGC (SEQ ID NO:62) and a reverse primer of sequence GACGGTGGGTACATGGCAAAC (SEQ ID NO:63). In some embodiments, CCND2 expression is determined using a probe of sequence CCTTCATTGCTCTGTGTGCCACCGAC (SEQ ID NO:64).

In some embodiments, CTBP2 expression is determined using isolated a polynucleotide comprising the sequence of ATCCGTGGGGAGACGCTG (SEQ ID NO:65), a polynucleotide comprising the sequence of CTCGAACTGCAACCGCCTG (SEQ ID NO:66), and a polynucleotide comprising the sequence of CCCGTGCGACCAAAGCCAATGAGG (SEQ ID NO:67), or complements thereof. In some embodiments, CTBP2 expression is determined using a forward primer of sequence ATCCGTGGGGAGACGCTG (SEQ ID NO:65) and a reverse primer of sequence of CTCGAACTGCAACCGCCTG (SEQ ID NO:66). In some embodiments, CTBP2 expression is determined using a probe of sequence CCCGTGCGACCAAAGCCAATGAGG (SEQ ID NO:67).

In some embodiments, DKK1 expression is determined using isolated a polynucleotide comprising the sequence of GACCATTGACAACTACCAGCCGTA (SEQ ID NO:68), a polynucleotide comprising the sequence of TGGGACTAGCGCAGTACTCATC (SEQ ID NO:69), and a polynucleotide comprising the sequence of TGCCGCACTCCTCGTCCTCTG (SEQ ID NO:70), or complements thereof. In some embodiments, DKK1 expression is determined using a forward primer of sequence GACCATTGACAACTACCAGCCGTA (SEQ ID NO:68) and a reverse primer of sequence of TGGGACTAGCGCAGTACTCATC (SEQ ID NO:69). In some embodiments, DKK1 expression is determined using a probe of sequence TGCCGCACTCCTCGTCCTCTG (SEQ ID NO:70).

In some embodiments, FBXW2 expression is determined using a polynucleotide comprising the sequence of GCCAGTTATGATATTCTCAGGGTCA (SEQ ID NO:71), a polynucleotide comprising the sequence of AGCAGGGCAAAGATATCTCCAAA (SEQ ID NO:72), and a polynucleotide comprising the sequence of AGACTCCTGAGATAGCAAACTTGGCCT (SEQ ID NO:73), or complements thereof. In some embodiments, FBXW2 expression is determined using a forward primer of sequence GCCAGTTATGATATTCTCAGGGTCA (SEQ ID NO:71) and a reverse primer of sequence AGCAGGGCAAAGATATCTCCAAA (SEQ ID NO:72). In some embodiments, FBXW2 expression is determined using a probe of sequence AGACTCCTGAGATAGCAAACTTGGCCT (SEQ ID NO:73).

In some embodiments, RHOU1 expression is determined using a polynucleotide comprising the sequence of CCCACCGAGTACATCCCTACTG (SEQ ID NO:74), a polynucleotide comprising the sequence of CAGTGTCACAGAGTTGGAGTCTCA (SEQ ID NO:75), and a polynucleotide comprising the sequence of CGCCCATCCACAGACACCACCG (SEQ ID NO:76), or complements thereof. In some embodiments, RHOU1 expression is determined using a forward primer of sequence CCCACCGAGTACATCCCTACTG (SEQ ID NO:74) and a reverse primer of sequence CAGTGTCACAGAGTTGGAGTCTCA (SEQ ID NO:75). In some embodiments, RHOU1 expression is determined using a probe of sequence CGCCCATCCACAGACACCACCG (SEQ ID NO:76).

In some embodiments, WIF1 expression is determined using a polynucleotide comprising the sequence of GTTCCAAAGGTTACCAGGGAGAC (SEQ ID NO:77), a polynucleotide comprising the sequence of GTTGGGTTCATGGCAGGTTCC (SEQ ID NO:78), and a polynucleotide comprising the sequence of CCAGGCTCGCAGACAGGCTTTGAAC (SEQ ID NO:79), or complements thereof. In some embodiments, WIF1 expression is determined using a forward primer of sequence GTTCCAAAGGTTACCAGGGAGAC (SEQ ID NO:77) and a reverse primer of sequence GTTGGGTTCATGGCAGGTTCC (SEQ ID NO:78). In some embodiments, WIF1 expression is determined using a probe of sequence CCAGGCTCGCAGACAGGCTTTGAAC (SEQ ID NO:79).

In some embodiments of any of the methods described herein, the expression levels of FBXW2, CCND2, RHOU, CTBP2, WIF1, and DKK1 are measured using polynucleotides selected from the group consisting of SEQ ID NOs:62-79. In some embodiments of any of the methods described herein, the expression levels of FBXW2, CCND2, RHOU, CTBP2, WIF1, and DKK1 are measured using (a) a forward primer of SEQ ID NO:62, a reverse primer of SEQ ID NO:63, and a probe comprising SEQ ID NO:64; (b) a forward primer of SEQ ID NO:65, a reverse primer of SEQ ID NO:66, and a probe comprising SEQ ID NO:67; (c) a forward primer of SEQ ID NO:68, a reverse primer of SEQ ID NO:69, and a probe comprising SEQ ID NO:70; (d) a forward primer of SEQ ID NO:71, a reverse primer of SEQ ID NO:72, and a probe comprising SEQ ID NO:73; (e) a forward primer of SEQ ID NO:74, a reverse primer of SEQ ID NO:75, and a probe comprising SEQ ID NO:76; and (f) a forward primer of SEQ ID NO:77, a reverse primer of SEQ ID NO:78, and a probe comprising SEQ ID NO:79.

In some embodiments, the expression level of each biomarker (e.g., FBXW2, CCND2, RHOU, CTBP2, WIF1, and/or DKK1) is determined in a separate assay (e.g., 6 assays). In some embodiments, the reference gene(s) and normalization methods for each assay are the same for all 6 assays. In some embodiments, the expression levels of several biomarkers (e.g., FBXW2, CCND2, RHOU, CTBP2, WIF1, and/or DKK1) are detected in a single multiplex assay.

Alternatively, biomarker expression levels may be determined by amplifying complementary DNA (cDNA) or complementary RNA (cRNA) produced from mRNA and analyzing it using a microarray. Microarray technology allows for simultaneous analysis of the expression of thousands of genes. A number of different array configurations and methods for their production are known to those skilled in the art. In addition, microarrays are commercially available (e.g., Affymetrix GeneChips) or can be custom-produced. Microarrays currently in wide use include cDNA arrays and oligonucleotide arrays. In general, polynucleotides of interest (e.g., probes or probe sets) are plated, or arrayed, on a microchip substrate. In some embodiments, probes to at least 10, 25, 50, 100, 500, 1000, 5000, 10,000, 20,000, or 25,000 or more genes are immobilized on an array substrate. The substrate may be a porous or nonporous support, such as a glass, plastic or gel surface. The probes can include DNA, RNA, copolymer sequences of DNA and RNA, DNA and/or RNA analogues, or combinations thereof. In some embodiments, a microarray includes a support with an ordered array of binding sites for each individual gene. The microarrays can be addressable arrays or positionally addressable arrays, e.g., each probe of the array is located at a known, predetermined position on the solid support such that the identity of each probe can be determined from its position of the array.

Each probe on the microarray can be between 10-50,000 nucleotides in length. In some embodiments, the probes of the microarray can consist of nucleotide sequences with lengths of less than about 1,000 nucleotides, less than about 750 nucleotides, less than about 500 nucleotides, less than about 250 nucleotides, less than about 100 nucleotides, or less than about 50 nucleotides in length. Generally, an array includes positive control probes and negative control probes.

In certain embodiments, the biomarker expression is determined using a microarray. For example, total RNA is extracted from a fresh frozen (FF) tissue sample or total RNA is extracted from a macro-dissected formalin-fixed paraffin embedded (FFPE) tissue sample. The quantity and quality of the total RNA is assessed by standard spectrophotometry and/or any other appropriate technology (e.g., an Agilent Bioanalyzer). Following RNA extraction, the RNA sample is amplified using standard methods and/or a commercially available amplification system (e.g., NuGEN Ovation RNA Amplification System V2). The amplified cDNA is fragmented, labeled, and hybridized to a microarray (e.g., using NuGEN Encore Biotin Module and Affymetrix GeneChip array) following standard procedures. The array is washed, stained, and scanned in accordance with the instructions for the microarray. The microarray data is pre-processed, the probe-level intensity measurements are background corrected, normalized, and summarized as expression measurements using the Robust Multichip algorithm (RMA). The probe level data is summarized to get the expression level of each biomarker (e.g., FBXW2, CCND2, RHOU, CTBP2, WIF1, and/or DKK1). A combination of quality parameter threshold and data reduction techniques (e.g., principal component analysis) is applied to the data set to establish profile quality and identify potential outlying samples. These normalized (or standardized) expression values for each biomarker are used to calculate the decision value of the sample.

In some embodiments, biomarker expression is analyzed by studying the protein expression of the gene or genes of interest. Commonly used methods for the analysis of protein expression, include but are not limited to, immunohistochemistry (IHC)-based, antibody-based, and mass spectrometry-based methods. Antibodies, generally monoclonal antibodies, may be used to detect expression of a gene product (e.g., protein). In some embodiments, the antibodies can be detected by direct labeling of the antibodies themselves. In other embodiments, an unlabeled primary antibody is used in conjunction with a labeled secondary antibody Immunohistochemistry methods and/or kits are well known in the art and are commercially available.

In some embodiments, biomarker expression is determined by an assay known to those of skill in the art, including but not limited to, multi-analyte profile test, enzyme-linked immunosorbent assay (ELISA), radioimmunoassay, Western blot assay, immunofluorescent assay, enzyme immunoassay, immunoprecipitation assay, chemiluminescent assay, immunohistochemical assay, dot blot assay or slot blot assay. In some embodiments, wherein an antibody is used in the assay the antibody is detectably labeled. The antibody labels may include, but are not limited to, immunofluorescent label, chemiluminescent label, phosphorescent label, enzyme label, radiolabel, avidin/biotin, colloidal gold particles, colored particles and magnetic particles.

Other suitable methods for analyzing biomarker expression include proteomics-based methods. Proteomics includes, among other things, study of the global changes of protein expression in a sample. In some embodiments, a proteomic method comprises the following steps: (1) separation of individual proteins in a sample by 2-D electrophoresis (2-D PAGE), (2) identification of individual proteins recovered from the gel (e.g., by mass spectrometry or N-terminal sequencing), and (3) analysis of the data using bioinformatics. In some embodiments, a proteomic method comprises using a tissue microarray (TMA). Tissue arrays may be constructed according to a variety of techniques known to one of skill in the art. In certain embodiments, a manual tissue arrayer is used to remove a “core” from a paraffin block prepared from a tissue sample. The core is then inserted into a separate paraffin block in a designated location on a grid. Cores from as many as about 400 samples can be inserted into a single recipient block. The resulting tissue array may be processed into thin sections for analysis. In some embodiments, a proteomic method comprises an antibody microarray. In some embodiments, a proteomic method comprises using mass spectrometry, including but not limited to, SELDI, MALDI, electro spray, and surface plasmon resonance methods. In some embodiments, a proteomic method comprises bead-based technology, including but not limited to, antibodies on beads in an array format. In some embodiments, the proteomic method comprises a reverse phase protein microarray (RPPM). In some embodiments, the proteomic method comprises multiplexed protein profiling, including but not limited to, the Global Proteome Survey (GPS) method.

In some embodiments, the biomarker signature is identified by differential gene expression between two samples. In some embodiments, the biomarker signature is identified by differential gene expression between two samples which comprise genes differentially expressed in cancer cells as compared to normal cells. In some embodiments, the biomarker signature comprises genes differentially expressed in tumorigenic cancer stem cells as compared to non-tumorigenic cancer cells. In some embodiments, the biomarker signature comprises genes differentially expressed in cells from a tumor which is responsive to a specific treatment as compared to cells from a tumor which is non-responsive to the same treatment.

In some embodiments, expression profiles are determined using microarray analysis. The microarray data identifies gene profiles comprising similarly and differentially expressed genes between two samples. In some embodiments, the expression profiles are refined, filtered, and/or subdivided into biomarker signatures based on fold expression change. In some embodiments, all genes above a certain fold expression change are included in the biomarker signature. The fold expression change may be elevated, reduced or both elevated and reduced. In some embodiments, all genes with a 2-fold or more expression change are included in the biomarker signature. In some embodiments, all genes with a 2.5-fold or more expression change are included in the biomarker signature. In some embodiments, all genes with a 3-fold or more expression change are included in the biomarker signature. In some embodiments, all genes with a 3.5-fold or more expression change are included in the biomarker signature. In some embodiments, all genes with a 4-fold or more expression change are included in the biomarker signature.

In some embodiments, the gene expression profiles are refined, filtered, and/or subdivided into biomarker signatures based on statistical analyses. The statistical methods may include, but are not limited to, cluster analysis, supported vector machines (SVM) analysis, supported vector machines-recursive feature elimination (SVM-RFE) analysis, Platt scaling, neural networks, and other algorithms. In some embodiments, the gene expression profiles are analyzed using a t-test analysis. In some embodiments, the gene expression profiles are analyzed using paired-sample empirical Baysian analysis. In some embodiments, a combination of statistical analyses is used. In some embodiments, SVM models are used to obtain decision values based on the training data. In some embodiments, the decision values are calculated by a weighted sum of the standardized expression of a set of biomarkers. In some embodiments, a positive decision value indicates a tumor predicted to be a responder while a negative decision value indicates a tumor predicted to be a non-responder. In some embodiments, classification probabilities for responders and non-responders are obtained using Platt scaling (Platt, 1999, Advances in Large Margin Classifiers, pp. 61-74, MIT Press). Platt scaling may comprise fitting a logistic distribution using maximum likelihood to decision values obtained, for example, by SVM models. In some embodiments, tumors associated with probabilities higher than 0.5 would be predicted to be a responder while tumors with probabilities lower than 0.5 would be predicted to be a non-responder.

In some embodiments of any of the methods or uses described herein, classification probabilities of a tumor (in regard to responder or non-responder status) are obtained based on the decision values. In some embodiments, the probabilities are obtained by fitting a logistic regression on the decision values. In some embodiments, tumors associated with probabilities higher than 0.5 are predicted to be a responder while tumors with probabilities lower than 0.5 are predicted to be a non-responder.

In some embodiments, a biomarker signature is obtained by a series of analytical steps. For example, expression data from a training set of samples are obtained from microarray analyses. The data are preprocessed to get an expression matrix with specific genes. Genes with near zero variance are removed, as are genes with expression values below a pre-determined level. The remaining genes are ranked using SVM-RFE analysis. Leave-one-out cross-validation (LOOCV) methods are used to identify and select the best predictive genes and also to measure positive predictive value (PPV), negative predictive value (NPV), sensitivity, and specificity.

In some embodiments, all genes with elevated expression, reduced expression, or both, with a P value across samples of 0.01 or less are included in the biomarker signature. In some embodiments, all genes with elevated expression, reduced expression or both, with a P value across samples of 0.005 or less are included in the biomarker signature. In some embodiments, all genes with elevated expression, reduced expression or both, with a P value across samples of 0.001 or less are included in the biomarker signature. In some embodiments, all genes with elevated expression, reduced expression or both, with a FDR (False Discovery Rate) of 0.25 or less are included in the biomarker signature. In some embodiments, all genes with elevated expression, reduced expression or both, with a FDR of 0.1 or less, 0.01 or less, or 0.001 or less are included in the biomarker signature.

In some embodiments, the gene expression profiles and/or biomarker signatures are refined, filtered, and/or subdivided based on statistical models. In some embodiments, the gene expression profiles and/or biomarker signatures are refined, filtered, and/or subdivided based on survival analysis models. These models may include, but are not limited to, Kaplan-Meier survival models, Cox proportional models, Cox proportional hazard models, chi-square analysis, univariate logistic regression models, multivariate competing risk models, linear discriminate analysis models, parametric regression models and correlation analysis models.

In some embodiments, the gene expression profiles and/or biomarker signatures are refined, filtered, subdivided and/or tested using gene expression array datasets that have associated clinical outcomes. There are several databases that contain datasets that are available to the public, for example, Gene Expression Omnibus (GEO) and ArrayExpress.

In some embodiments, the gene expression profiles and/or biomarker signatures are refined using biological function parameters, and/or gene sets. For example, in some embodiments, gene expression profiles, and/or biomarker signatures are refined using Gene Set Enrichment Analysis (GSEA) (Subramanian et al., 2005, PNAS, 102: 15545-15550). In some embodiments, the gene expression profiles are refined based on their ability to predict clinical outcome.

In some of the embodiments of the methods described herein, the Wnt pathway inhibitor is an anti-FZD antibody as described herein. In some of the embodiments of the methods described herein, the Wnt pathway inhibitor is an antibody that specifically binds at least one Frizzled (FZD) protein or portion thereof. In some embodiments, the anti-FZD antibody specifically binds at least one FZD protein selected from the group consisting of: FZD1, FZD2, FZD5, FZD7, and FZD8. In other embodiments, the anti-FZD antibody comprises: (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6). In some embodiments, the anti-FZD antibody comprises a heavy chain variable region comprising the amino acids of SEQ ID NO:7. In some embodiments, the anti-FZD antibody comprises a light chain variable region comprising the amino acids of SEQ ID NO:8. In some embodiments, the anti-FZD antibody comprises a heavy chain variable region comprising the amino acids of SEQ ID NO:7 and a light chain variable region comprising the amino acids of SEQ ID NO:8. In some embodiments, the anti-FZD antibody is antibody OMP-18R5. In some embodiments, the anti-FZD antibody is encoded by the plasmid having ATCC deposit no. PTA-9541. In other embodiments, the anti-FZD antibody competes for specific binding to at least one human FZD protein with an antibody encoded by the plasmid deposited with ATCC having deposit no. PTA-9541.

In some embodiments of the methods described herein, the tumor is selected from the group consisting of a breast tumor, lung tumor, a colon tumor, glioma, a gastrointestinal tumor, a renal tumor, an ovarian tumor, a liver tumor, a colorectal tumor, an endometrial tumor, a kidney tumor, a prostate tumor, a thyroid tumor, a neuroblastoma, a pancreatic tumor, a glioblastoma multiforme, a cervical tumor, a stomach tumor, a bladder tumor, a hepatoma, melanoma, and a head and neck tumor. In some embodiments, the tumor is a breast tumor. In some embodiments, the tumor is a HER2-negative breast tumor. In some embodiments, the tumor is a triple negative breast cancer (TNBC) tumor.

In some embodiments of the methods described herein, the cancer is selected from the group consisting of a breast cancer, lung cancer, a colon cancer, glioma, a gastrointestinal cancer, a renal cancer, an ovarian cancer, a liver cancer, a colorectal cancer, an endometrial cancer, a kidney cancer, a prostate cancer, a thyroid cancer, a neuroblastoma, a pancreatic cancer, a glioblastoma multiforme, a cervical cancer, a stomach cancer, a bladder cancer, a hepatoma, melanoma, and a head and neck cancer. In some embodiments, the cancer is breast cancer. In some embodiments, the cancer is a HER2-negative breast cancer. In some embodiments, the cancer is a triple negative breast cancer (TNBC).

In some of the embodiments of the methods described herein, the method comprises treating a patient with a Wnt pathway inhibitor described herein (e.g., an anti-FZD antibody), particularly after the patient has been identified as being responsive to treatment with the Wnt pathway inhibitor. In some embodiments, the treatment comprises administering at least one additional therapeutic agent in combination with the Wnt pathway inhibitor. An additional therapeutic agent can be administered prior to, concurrently with, and/or subsequently to, administration of the Wnt pathway inhibitor. In some embodiments, the at least one additional therapeutic agent comprises 1, 2, 3, or more additional therapeutic agents.

Useful classes of therapeutic agents include, for example, antitubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Therapeutic agents that may be administered in combination with the Wnt pathway inhibitors include chemotherapeutic agents. Thus, in some embodiments, the method or treatment involves the administration of a Wnt pathway inhibitor of the present invention in combination with a chemotherapeutic agent or cocktail of multiple different chemotherapeutic agents. Treatment with a Wnt pathway inhibitor (e.g., an anti-FZD antibody) can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4th Edition, 2008, M. C. Perry, Editor, Lippincott, Williams & Wilkins, Philadelphia, PA.

Chemotherapeutic agents useful in the instant invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is paclitaxel (taxol).

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HC1, daunorubicin citrate, mitoxantrone HC1, actinomycin D, etoposide, topotecan HC1, teniposide (VM-26), and irinotecan, as well as pharmaceutically acceptable salts, acids, or derivatives of any of these.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (nab-paclitaxel; ABRAXANE), DHA-paclitaxel, or PG-paclitaxel. In certain alternative embodiments, the antimitotic agent comprises a vinca alkaloid, such as vincristine, binblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, where the chemotherapeutic agent administered in combination with a Wnt pathway inhibitor is an anti-mitotic agent, the cancer or tumor being treated is breast cancer or a breast tumor. In certain embodiments, the additional therapeutic agent is paclitaxel (taxol) or albumin-bound paclitaxel.

In some embodiments, an additional therapeutic agent comprises an agent such as a small molecule. For example, treatment can involve the combined administration of a Wnt pathway inhibitor of the present invention with a small molecule that acts as an inhibitor against additional tumor-associated antigens including, but not limited to, EGFR, ErbB2, HER2, and/or VEGF. In certain embodiments, the additional therapeutic agent is a small molecule that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway.

Certain embodiments of the present invention comprise a method of identifying a human breast tumor that is likely to be responsive to or non-responsive to treatment with an antibody that specifically binds at least one human frizzled (FZD) selected from the group consisting of FZD 1, FZD2, FZD5, FZD7, and FZD8, the method comprising (a) obtaining a sample of the human breast tumor; (b) measuring the expression level of each biomarker of a biomarker signature in the sample, wherein the biomarker signature comprises one or more of the biomarkers FBXW2, CCND2, RHOU, CTBP2, WIF1, and DKK1; and (c) calculating a decision value based upon the standardized expression of the biomarkers in the biomarker signature; wherein a positive decision value indicates the breast tumor is predicted to be responsive to treatment with the antibody and a negative decision value indicates the tumor is predicted to be non-responsive to treatment with the antibody. Some embodiments comprise a method of identifying a patient with breast cancer that is likely to be responsive to treatment with an antibody that specifically binds at least one human frizzled (FZD) selected from the group consisting of FZD1, FZD2, FZD5, FZD7, and FZD8, the method comprising: (a) obtaining a sample of the breast cancer; (b) measuring the expression level of each biomarker of a biomarker signature in the sample, wherein the biomarker signature comprises one or more of the biomarkers FBXW2, CCND2, RHOU, CTBP2, WIF1, and DKK1; and (c) calculating a decision value based upon the standardized expression of the biomarkers in the biomarker signature; wherein a positive decision value indicates the breast cancer is predicted to be responsive to treatment with the antibody. Some embodiments comprise a method of selecting a patient with breast cancer for treatment with an antibody that specifically binds at least one human frizzled (FZD) selected from the group consisting of FZD1, FZD2, FZD5, FZD7, and FZD8, the method comprising: (a) obtaining a sample of the breast cancer; (b) measuring the expression level of each biomarker of a biomarker signature in the sample, wherein the biomarker signature comprises one or more of the biomarkers FBXW2, CCND2, RHOU, CTBP2, WIF1, and DKK1; (c) calculating a decision value based upon the standardized expression of the biomarkers in the biomarker signature; wherein a positive decision value indicates the breast cancer is predicted to be responsive to treatment with the antibody; and selecting the patient for treatment when their tumor sample has a positive decision value.

Some embodiments of the present invention comprise a method of treating breast cancer in a patient, comprising: (a) identifying if the patient is likely to respond to treatment with an antibody that specifically binds at least one human frizzled (FZD) selected from the group consisting of FZD 1, FZD2, FZD5, FZD7, and FZD8, wherein the identification comprises: (i) obtaining a sample of the patient's breast cancer; (ii) measuring the expression level of each biomarker of a biomarker signature in the sample, wherein the biomarker signature comprises one or more of the biomarkers FBXW2, CCND2, RHOU, CTBP2, WIF1, and DKK1; and (iii) calculating a decision value based upon the standardized expression of the biomarkers in the signature; wherein a positive decision value indicates that the patient is predicted to respond to treatment; and (b) administering to the patient who is predicted to response to treatment an effective amount of the antibody.

Certain embodiments of the present invention comprise a method of identifying a human breast tumor that is likely to be responsive to or non-responsive to treatment with anti-FZD antibody OMP-18R5 in combination with paclitaxel, the method comprising (a) obtaining a sample of the human breast tumor; (b) measuring the expression level of each biomarker of a biomarker signature in the sample, wherein the biomarker signature comprises the biomarkers FBXW2, CCND2, RHOU, CTBP2, WIF1, and DKK1; and (c) calculating a decision value based upon the standardized expression of the biomarkers in the biomarker signature; wherein a positive decision value indicates the breast tumor is predicted to be responsive to treatment and a negative decision value indicates the tumor is predicted to be non-responsive to treatment. Some embodiments comprise a method of identifying a patient with breast cancer that is likely to be responsive to treatment with the anti-FZD antibody OMP-18R5 in combination with paclitaxel, the method comprising: (a) obtaining a sample of the breast cancer; (b) measuring the expression level of each biomarker of a biomarker signature in the sample, wherein the biomarker signature comprises the biomarkers FBXW2, CCND2, RHOU, CTBP2, WIF1, and DKK1; and (c) calculating a decision value based upon the standardized expression of the biomarkers in the biomarker signature; wherein a positive decision value indicates the breast cancer is predicted to be responsive to treatment. Some embodiments comprise a method of selecting a patient with breast cancer for treatment with the anti-FZD antibody OMP-18R5 in combination with paclitaxel, the method comprising: (a) obtaining a sample of the breast cancer; (b) measuring the expression level of each biomarker of a biomarker signature in the sample, wherein the biomarker signature comprises the biomarkers FBXW2, CCND2, RHOU, CTBP2, WIF1, and DKK1; (c) calculating a decision value based upon the standardized expression of the biomarkers in the biomarker signature; wherein a positive decision value indicates the breast cancer is predicted to be responsive to treatment; and selecting the patient for treatment when their tumor sample has a positive decision value.

Some embodiments of the present invention comprise a method of treating breast cancer in a patient, comprising: (a) identifying if the patient is likely to respond to treatment with the anti-FZD antibody OMP-18R5 in combination with paclitaxel, wherein the identification comprises: (i) obtaining a sample of the patient's breast cancer; (ii) measuring the expression level of each biomarker of a biomarker signature in the sample, wherein the biomarker signature comprises the biomarkers FBXW2, CCND2, RHOU, CTBP2, WIF1, and DKK1; and (iii) calculating a decision value based upon the standardized expression of the biomarkers in the signature; wherein a positive decision value indicates that the patient is predicted to respond to treatment; and (b) administering to the patient who is predicted to response to treatment an effective amount of the antibody and paclitaxel.

### III. Wnt Pathway Inhibitors

The present invention provides methods for identifying tumors and/or patients with cancer that are likely to be responsive to or sensitive to treatment with Wnt pathway inhibitors. As used herein “Wnt pathway inhibitor” includes, but is not limited to, Frizzled (FZD) binding agents and Wnt-binding agents. FZD-binding agents may include antibodies that specifically bind to FZD proteins. Wnt-binding agents may include antibodies that specifically bind to Wnt proteins as well as soluble FZD receptors that bind to Wnt proteins.

In certain embodiments, the Wnt pathway inhibitors are agents that bind one or more human FZD proteins. In some embodiments, the FZD-binding agents specifically bind one, two, three, four, five, six, seven, eight, nine, or ten FZD proteins. In some embodiments, the FZD-binding agent binds one or more FZD proteins selected from the group consisting of FZD1, FZD2, FZD3, FZD4, FZD5, FZD6, FZD7, FZD8, FZD9, and FZD10. In some embodiments, FZD-binding agent binds one or more FZD proteins comprising FZD1, FZD2, FZD5, FZD7, and/or FZD8. In certain embodiments, FZD-binding agent binds FZD7. In certain embodiments, FZD-binding agent binds FZD5 and/or FZD8. In certain embodiments, the FZD-binding agent specifically binds FZD1, FZD2, FZD5, FZD7, and FZD8. Non-limiting examples of FZD-binding agents can be found in U.S. Pat. No. 7,982,013.

In certain embodiments, the FZD-binding agent is a FZD antagonist. In certain embodiments, the FZD-binding agent is a Wnt pathway antagonist. In certain embodiments, the FZD-binding agent inhibits Wnt signaling. In some embodiments, the FZD-binding agent inhibits canonical Wnt signaling.

In some embodiments, the FZD-binding agents are antibodies. In some embodiments, the FZD-binding agents are polypeptides. In certain embodiments, the FZD-binding agent is an antibody or a polypeptide comprising an antigen-binding site. In certain embodiments, an antigen-binding site of a FZD-binding antibody or polypeptide described herein is capable of binding (or binds) one, two, three, four, five, or more human FZD proteins. In certain embodiments, an antigen-binding site of the FZD-binding antibody or polypeptide is capable of specifically binding one, two, three, four, or five human FZD proteins selected from the group consisting of FZD1, FZD2, FZD3, FZD4, FZD5, FZD6, FZD7, FZD8, FZD9 and FZD10. In some embodiments, when the FZD-binding agent is an antibody that binds more than one FZD protein, it may be referred to as a “pan-FZD antibody”.

In certain embodiments, the FZD-binding agent (e.g., antibody) specifically binds the extracellular domain (ECD) of the one or more human FZD proteins to which it binds. In certain embodiments, the FZD-binding agent specifically binds within the Fri domain (also known as the cysteine-rich domain (CRD)) of the human FZD protein to which it binds. Sequences of the Fri domain of each of the human FZD proteins are known in the art and are provided as SEQ ID NO:13 (FZD1), SEQ ID NO:14 (FZD2), SEQ ID NO:15 (FZD3), SEQ ID NO:16 (FZD4), SEQ ID NO:17 (FZD5), SEQ ID NO:18 (FZD6), SEQ ID NO:19 (FZD7), SEQ ID NO:20 (FZD), SEQ ID NO:21 (FZD9), and SEQ ID NO:22 (FZD10).

In certain embodiments, the FZD-binding agent binds one, two, three, four, five, or more FZD proteins. In some embodiments, the FZD-binding agent specifically binds one, two, three, four, or five FZD proteins selected from the group consisting of FZD1, FZD2, FZD5, FZD7, and FZD8. In some embodiments, the FZD-binding agent specifically binds at least FZD5 and FZD8.

In some embodiments, the FZD-binding agent binds at least one human FZD protein with a dissociation constant (K_{D}) of about 1 μM or less, about 100 nM or less, about 40 nM or less, about 20 nM or less, about 10 nM or less, about 1 nM or less, or about 0.1 nM or less. In some embodiments, a FZD-binding agent binds at least one FZD protein with a K_{D} of about 10 nM or less. In some embodiments, a FZD-binding agent binds at least one FZD protein with a K_{D} of about 1 nM or less. In some embodiments, a FZD-binding agent binds at least one FZD protein with a K_{D} of about 0.1 nM or less. In certain embodiments, a FZD-binding agent binds each of one or more (e.g., 1, 2, 3, 4, or 5) of FZD1, FZD2, FZD5, FZD7, and FZD8 with a K_{D} of about 40 nM or less. In certain embodiments, the FZD-binding agent binds to each of one or more of FZD 1, FZD2, FZD5, FZD7, and FZD8 with a K_{D} of about 10 nM or less. In certain embodiments, the FZD-binding agent binds each of FZD 1, FZD2, FZD5, FZD7, and FZD8 with a K_{D} of about 10 nM. In some embodiments, the K_{D} of the binding agent (e.g., an antibody) to a FZD protein is the K_{D} determined using a FZD-Fc fusion protein comprising at least a portion of the FZD extracellular domain or FZD-Fri domain immobilized on a Biacore chip.

In certain embodiments, the FZD-binding agent binds one or more (for example, two or more, three or more, or four or more) human FZD proteins with an EC₅₀ of about 1 μM or less, about 100 nM or less, about 40 nM or less, about 20 nM or less, about 10 nM or less, or about 1 nM or less. In certain embodiments, a FZD-binding agent binds to more than one FZD protein with an EC₅₀ of about 40 nM or less, about 20 nM or less, or about 10 nM or less. In certain embodiments, the FZD-binding agent has an EC₅₀ of about 20 nM or less with respect to one or more (e.g., 1, 2, 3, 4, or 5) of the following FZD proteins: FZD1, FZD2, FZD5, FZD7, and FZD8. In certain embodiments, the FZD-binding agent has an EC₅₀ of about 10 nM or less with respect to one or more (e.g., 1, 2, 3, 4, or 5) of the following FZD proteins: FZD1, FZD2, FZD5, FZD7, and FZD8. In certain embodiments, the FZD-binding agent has an EC₅₀ of about 40 nM or less or 20 nM or less with respect to binding of FZD5 and/or FZD8.

In certain embodiments, the Wnt pathway inhibitor is a FZD-binding agent which is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is monovalent, monospecific, or bivalent. In some embodiments, the antibody is a bispecific antibody or a multispecific antibody. In some embodiments, the antibody is conjugated to a cytotoxic moiety. In some embodiments, the antibody is isolated. In some embodiments, the antibody is substantially pure.

The FZD-binding agents (e.g., antibodies) of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blot analysis, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well-known in the art (see, e.g., Ausubel et al., Editors, 1994-present, Current Protocols in Molecular Biology, John Wiley & Sons, Inc., New York, N.Y.).

In certain embodiments, the invention provides a Wnt pathway inhibitor which is a FZD-binding agent (e.g., an antibody) that comprises a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3). In some embodiments, the FZD-binding agent further comprises a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6). In some embodiments, the FZD-binding agent comprises a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6). In certain embodiments, the FZD-binding agent comprises: (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6).

In certain embodiments, the invention provides a FZD-binding agent (e.g., an antibody) that comprises: (a) a heavy chain CDR1 comprising GFTFSHYTLS (SEQ ID NO:1), or a variant thereof comprising 1, 2, 3, or 4 amino acid substitutions; (b) a heavy chain CDR2 comprising VISGDGSYTYYADSVKG (SEQ ID NO:2), or a variant thereof comprising 1, 2, 3, or 4 amino acid substitutions; (c) a heavy chain CDR3 comprising NFIKYVFAN (SEQ ID NO:3), or a variant thereof comprising 1, 2, 3, or 4 amino acid substitutions; (d) a light chain CDR1 comprising SGDNIGSFYVH (SEQ ID NO:4), or a variant thereof comprising 1, 2, 3, or 4 amino acid substitutions; (e) a light chain CDR2 comprising DKSNRPSG (SEQ ID NO:5), or a variant thereof comprising 1, 2, 3, or 4 amino acid substitutions; and (f) a light chain CDR3 comprising QSYANTLSL (SEQ ID NO:6), or a variant thereof comprising 1, 2, 3, or 4 amino acid substitutions. In certain embodiments, the amino acid substitutions are conservative substitutions.

In certain embodiments, the invention provides a FZD-binding agent (e.g., an antibody) that comprises a heavy chain variable region having at least about 80% sequence identity to SEQ ID NO:7, and/or a light chain variable region having at least 80% sequence identity to SEQ ID NO:8. In certain embodiments, the FZD-binding agent comprises a heavy chain variable region having at least about 85%, at least about 90%, at least about 95%, at least about 97%, or at least about 99% sequence identity to SEQ ID NO:7. In certain embodiments, the FZD-binding agent comprises a light chain variable region having at least about 85%, at least about 90%, at least about 95%, at least about 97%, or at least about 99% sequence identity to SEQ ID NO:8. In certain embodiments, the FZD-binding agent comprises a heavy chain variable region having at least about 95% sequence identity to SEQ ID NO:7, and/or a light chain variable region having at least about 95% sequence identity to SEQ ID NO:8. In certain embodiments, the FZD-binding agent comprises a heavy chain variable region comprising SEQ ID NO:7 and/or a light chain variable region comprising SEQ ID NO:8. In certain embodiments, the FZD-binding agent comprises a heavy chain variable region comprising SEQ ID NO:7 and a light chain variable region comprising SEQ ID NO:8. In certain embodiments, the FZD-binding agent comprises a heavy chain variable region consisting essentially of SEQ ID NO:7 and a light chain variable region consisting essentially of SEQ ID NO:8.

In certain embodiments, the invention provides a FZD-binding agent (e.g., an antibody) that comprises: (a) a heavy chain having at least 90% sequence identity to SEQ ID NO:9 (with or without the signal sequence) or SEQ ID NO:11; and/or (b) a light chain having at least 90% sequence identity to SEQ ID NO:10 (with or without the signal sequence) or SEQ ID NO:12. In some embodiments, the FZD-binding agent comprises: (a) a heavy chain having at least 95% sequence identity to SEQ ID NO:9 (with or without the signal sequence) or SEQ ID NO:11; and/or (b) a light chain having at least 95% sequence identity to SEQ ID NO:10 (with or without the signal sequence) or SEQ ID NO:12. In some embodiments, the FZD-binding agent comprises a heavy chain comprising SEQ ID NO:9 (with or without the signal sequence) or SEQ ID NO:11, and/or a light chain comprising SEQ ID NO:10 (with or without the signal sequence) or SEQ ID NO:12. In some embodiments, the FZD-binding agent comprises a heavy chain comprising SEQ ID NO:11 and a light chain comprising SEQ ID NO:12. In some embodiments, the FZD-binding agent comprises a heavy chain consisting essentially of amino acids 20-463 of SEQ ID NO:9 and a light chain consisting essentially of amino acids 20-232 of SEQ ID NO:10. In some embodiments, the FZD-binding agent comprises a heavy chain consisting essentially of SEQ ID NO:11 and a light chain consisting essentially of SEQ ID NO:12.

In certain embodiments, the invention provides a Wnt pathway inhibitor which is a FZD-binding agent (e.g., an antibody) that specifically binds at least one of FZD1, FZD2, FZD5, FZD7, and/or FZD8, wherein the FZD-binding agent (e.g., an antibody) comprises one, two, three, four, five, and/or six of the CDRs of antibody OMP-18R5. Antibody OMP-18R5 (also known as 18R5 and vantictumab), as well as other FZD-binding agents, has been previously described in U.S. Pat. No. 7,982,013. DNA encoding the heavy chain and light chain of the OMP-18R5 IgG2 antibody was deposited with the ATCC, under the conditions of the Budapest Treaty on Sep. 29, 2008, and assigned ATCC deposit designation number PTA-9541. In some embodiments, the FZD-binding agent comprises one or more of the CDRs of OMP-18R5, two or more of the CDRs of OMP-18R5, three or more of the CDRs of OMP-18R5, four or more of the CDRs of OMP-18R5, five or more of the CDRs of OMP-18R5, or all six of the CDRs of OMP-18R5.

The invention provides polypeptides which are Wnt pathway inhibitors. The polypeptides include, but are not limited to, antibodies that specifically bind human FZD proteins. In some embodiments, a polypeptide binds one or more FZD proteins selected from the group consisting of FZD1, FZD2, FZD3, FZD4, FZD5, FZD6, FZD7, FZD8, FZD9, and FZD10. In some embodiments, a polypeptide binds FZD1, FZD2, FZD5, FZD7, and/or FZD8. In some embodiments, a polypeptide binds FZD1, FZD2, FZD5, FZD7, and FZD8.

In certain embodiments, a polypeptide comprises one, two, three, four, five, and/or six of the CDRs of antibody OMP-18R5. In some embodiments, a polypeptide comprises CDRs with up to four (i.e., 0, 1, 2, 3, or 4) amino acid substitutions per CDR. In certain embodiments, the heavy chain CDR(s) are contained within a heavy chain variable region. In certain embodiments, the light chain CDR(s) are contained within a light chain variable region.

In some embodiments, the invention provides a polypeptide that specifically binds one or more human FZD proteins, wherein the polypeptide comprises an amino acid sequence having at least about 80% sequence identity to SEQ ID NO:7, and/or an amino acid sequence having at least about 80% sequence identity to SEQ ID NO:8. In certain embodiments, the polypeptide comprises an amino acid sequence having at least about 85%, at least about 90%, at least about 95%, at least about 97%, or at least about 99% sequence identity to SEQ ID NO:7. In certain embodiments, the polypeptide comprises an amino acid sequence having at least about 85%, at least about 90%, at least about 95%, at least about 97%, or at least about 99% sequence identity to SEQ ID NO:8. In certain embodiments, the polypeptide comprises an amino acid sequence having at least about 95% sequence identity to SEQ ID NO:7, and/or an amino acid sequence having at least about 95% sequence identity to SEQ ID NO:8. In certain embodiments, the polypeptide comprises an amino acid sequence comprising SEQ ID NO:7, and/or an amino acid sequence comprising SEQ ID NO:8.

In some embodiments, a FZD-binding agent comprises a polypeptide comprising a sequence selected from the group consisting of: SEQ ID NO:7, SEQ ID NO:8, SEQ ID NO:9, SEQ ID NO:10, SEQ ID NO:11, and SEQ ID NO:12.

In certain embodiments, a FZD-binding agent comprises the heavy chain variable region and light chain variable region of the OMP-18R5 antibody. In certain embodiments, a FZD-binding agent comprises the heavy chain and light chain of the OMP-18R5 antibody (with or without the leader sequence).

In certain embodiments, a FZD-binding agent comprises, consists essentially of, or consists of, the antibody OMP-18R5.

In certain embodiments, a FZD-binding agent (e.g., antibody) competes for specific binding to one or more human FZD proteins with an antibody that comprises a heavy chain variable region comprising SEQ ID NO:7 and a light chain variable region comprising SEQ ID NO:8. In certain embodiments, a FZD-binding agent (e.g., antibody) competes for specific binding to one or more human FZD proteins with an antibody that comprises a heavy chain comprising SEQ ID NO:9 (with or without the signal sequence) and a light chain comprising SEQ ID NO:10 (with or without the signal sequence). In certain embodiments, a FZD-binding agent (e.g., antibody) competes for specific binding to one or more human FZD proteins with an antibody that comprises a heavy chain comprising SEQ ID NO:11 and a light chain comprising SEQ ID NO:12. In certain embodiments, a FZD-binding agent competes with antibody OMP-18R5 for specific binding to one or more human FZD proteins. In some embodiments, a FZD-binding agent or antibody competes for specific binding to one or more human FZD proteins in an in vitro competitive binding assay.

In certain embodiments, a FZD-binding agent (e.g., an antibody) binds the same epitope, or essentially the same epitope, on one or more human FZD proteins as an antibody of the invention. In another embodiment, a FZD-binding agent is an antibody that binds an epitope on one or more human FZD proteins that overlaps with the epitope on a FZD protein bound by an antibody of the invention. In certain embodiments, a FZD-binding agent (e.g., an antibody) binds the same epitope, or essentially the same epitope, on one or more FZD proteins as antibody OMP-18R5. In another embodiment, the FZD-binding agent is an antibody that binds an epitope on one or more human FZD proteins that overlaps with the epitope on a FZD protein bound by antibody OMP-18R5.

In certain embodiments, the Wnt pathway inhibitors are agents that bind one or more human Wnt proteins. In certain embodiments, the agents specifically bind one, two, three, four, five, six, seven, eight, nine, ten, or more Wnt proteins. In some embodiments, the Wnt-binding agents bind one or more human Wnt proteins selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In certain embodiments, a Wnt-binding agent binds one or more (or two or more, three or more, four or more, five or more, etc.) Wnt proteins selected from the group consisting of Wnt1, Wnt2, Wnt2b, Wnt3, Wnt3a, Wnt7a, Wnt7b, Wnt8a, Wnt8b, Wnt10a, and Wnt10b. In certain embodiments, the one or more (or two or more, three or more, four or more, five or more, etc.) Wnt proteins are selected from the group consisting of Wnt1, Wnt2, Wnt2b, Wnt3, Wnt3a, Wnt8a, Wnt8b, Wnt10a, and Wnt10b.

In certain embodiments, the Wnt-binding agent is a Wnt antagonist. In certain embodiments, the Wnt-binding agent is a Wnt pathway antagonist. In certain embodiments, the Wnt-binding agent inhibits Wnt signaling. In some embodiments, the Wnt-binding agent inhibits canonical Wnt signaling.

In some embodiments, the Wnt-binding agent is an antibody. In some embodiments, the Wnt-binding agent is a polypeptide. In certain embodiments, the Wnt-binding agent is an antibody or a polypeptide comprising an antigen-binding site. In certain embodiments, an antigen-binding site of a Wnt-binding antibody or polypeptide described herein is capable of binding (or binds) one, two, three, four, five, or more human Wnt proteins. In certain embodiments, an antigen-binding site of the Wnt-binding antibody or polypeptide is capable of specifically binding one, two, three, four, or five human Wnt proteins selected from the group consisting of Wnt1, Wnt2, Wnt2b, Wnt3, Wnt3a, Wnt7a, Wnt7b, Wnt8a, Wnt8b, Wnt10a, and Wnt10b. Non-limiting examples of Wnt-binding agents can be found in International Publication WO 2011/088127.

In certain embodiments, a Wnt-binding agent binds to the C-terminal cysteine rich domain of one or more human Wnt proteins. In certain embodiments, the Wnt-binding agent binds a domain within the one or more Wnt proteins selected from the group consisting of: SEQ ID NO:46 (Wnt1), SEQ ID NO:47 (Wnt2), SEQ ID NO:48 (Wnt2b), SEQ ID NO:49 (Wnt3), SEQ ID NO:50 (Wnt3a), SEQ ID NO:51 (Wnt7a), SEQ ID NO:52 (Wnt7b), SEQ ID NO:53 (Wnt8a), SEQ ID NO:54 (Wnt8b), SEQ ID NO:55 (Wnt10a), and SEQ ID NO:56 (Wnt10b).

In certain embodiments, the Wnt-binding agent binds one or more (e.g., two or more, three or more, or four or more) Wnt proteins with a K_{D} of about 1 μM or less, about 100 nM or less, about 40 nM or less, about 20 nM or less, or about 10 nM or less. For example, in certain embodiments, a Wnt-binding agent described herein that binds more than one Wnt protein, binds those Wnt proteins with a K_{D} of about 100 nM or less, about 20 nM or less, or about 10 nM or less. In certain embodiments, the Wnt-binding agent binds each of one or more (e.g., 1, 2, 3, 4, or 5) Wnt proteins with a K_{D} of about 40 nM or less, wherein the Wnt proteins are selected from the group consisting of: Wnt1, Wnt2, Wnt2b, Wnt3, Wnt3a, Wnt7a, Wnt7b, Wnt8a, Wnt8b, Wnt10a, and Wnt10b. In some embodiments, the K_{D} of the binding agent (e.g., an antibody) to a Wnt protein is the K_{D} determined using a Wnt fusion protein comprising at least a portion of the Wnt C-terminal cysteine rich domain immobilized on a Biacore chip.

In certain embodiments, the Wnt-binding agent binds one or more (for example, two or more, three or more, or four or more) human Wnt proteins with an EC₅₀ of about 1 μM or less, about 100 nM or less, about 40 nM or less, about 20 nM or less, about 10 nM or less, or about 1 nM or less. In certain embodiments, a Wnt-binding agent binds to more than one Wnt with an EC₅₀ of about 40 nM or less, about 20 nM or less, or about 10 nM or less. In certain embodiments, the Wnt-binding agent has an EC₅₀ of about 20 nM or less with respect to one or more (e.g., 1, 2, 3, 4, or 5) of Wnt proteins Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and/or Wnt16. In certain embodiments, the Wnt-binding agent has an EC₅₀ of about 10 nM or less with respect to one or more (e.g., 1, 2, 3, 4, or 5) of the following Wnt proteins Wnt1, Wnt2, Wnt2b, Wnt3, Wnt3a, Wnt8a, Wnt8b, Wnt10a, and/or Wnt10b.

In certain embodiments, the Wnt pathway inhibitor is a Wnt-binding agent which is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is monovalent, monospecific, or bivalent. In some embodiments, the antibody is a bispecific antibody or a multispecific antibody. In some embodiments, the antibody is conjugated to a cytotoxic moiety. In some embodiments, the antibody is isolated. In some embodiments, the antibody is substantially pure.

The Wnt-binding agents (e.g., antibodies) of the present invention can be assayed for specific binding by any method known in the art as described herein for FZD-binding agents.

In certain embodiments, the Wnt-binding agent is a soluble receptor. In certain embodiments, the Wnt-binding agent comprises the extracellular domain of a FZD receptor protein. In some embodiments, the Wnt-binding agent comprises a Fri domain of a FZD protein. In some embodiments, a soluble receptor comprising a FZD Fri domain can demonstrate altered biological activity (e.g., increased protein half-life) compared to a soluble receptor comprising the entire FZD ECD. Protein half-life can be further modified (i.e., increased) by covalent modification with polyethylene glycol (PEG) or polyethylene oxide (PEO). In certain embodiments, the FZD protein is a human FZD protein. In certain embodiments, the human FZD protein is FZD1, FZD2, FZD3, FZD4, FZD5, FZD6, FZD7, FZD8, FZD9, or FZD10. Non-limiting examples of soluble FZD receptors can be found in U.S. Pat. Nos. 7,723,477 and 7,947,277 and U.S. Patent Publication No. 2013/0034551.

The predicted Fri domains for each of the human FZD1-10 proteins are provided as SEQ ID NOs:13-22. The predicted minimal Fri domains for each of the human FZD1-10 proteins are provided as SEQ ID NOs:23-32. Those of skill in the art may differ in their understanding of the exact amino acids corresponding to the various Fri domains. Thus, the N-terminus and/or C-terminus of the domains outlined above and herein may extend or be shortened by 1, 2, 3, 4, 5, 6, 7, 8, 9, or even 10 amino acids.

In certain embodiments, the Wnt-binding agent comprises a Fri domain of a human FZD protein, or a fragment or variant of the Fri domain that binds one or more human Wnt proteins. In certain embodiments, the human FZD protein is FZD1, FZD2, FZD3, FZD4, FZD5, FZD6, FZD7, FZD8, FZD9, or FZD 10. In certain embodiments, the human FZD protein is FZD4. In certain embodiments, the human FZD protein is FZD5. In certain embodiments, the human FZD protein is FZD8. In certain embodiments, the human FZD protein is FZD10. In certain embodiments, the FZD protein is FZD4 and the Wnt-binding agent comprises SEQ ID NO:16. In certain embodiments, the FZD protein is FZD5 and the Wnt-binding agent comprises SEQ ID NO:17. In certain embodiments, the FZD protein is FZD7 and the Wnt-binding agent comprises SEQ ID NO:19. In certain embodiments, the FZD protein is FZD8 and the Wnt-binding agent comprises SEQ ID NO:20. In certain embodiments, the FZD protein is FZD 10 and the Wnt-binding agent comprises SEQ ID NO:22. In certain embodiments, the FZD protein is FZD8 and the Wnt-binding agent comprises SEQ ID NO:33.

In some embodiments, the Wnt-binding agent comprises a Fri domain comprising the minimal Fri domain of FZD1 (SEQ ID NO:23), the minimal Fri domain of FZD2 (SEQ ID NO:24), the minimal Fri domain of FZD3 (SEQ ID NO:25), the minimal Fri domain of FZD4 (SEQ ID NO:26), the minimal Fri domain of FZD5 (SEQ ID NO:27), the minimal Fri domain of FZD6 (SEQ ID NO:28), the minimal Fri domain of FZD7 (SEQ ID NO:29), the minimal Fri domain of FZD8 (SEQ ID NO:30), the minimal Fri domain of FZD9 (SEQ ID NO:31), or the minimal Fri domain of FZD10 (SEQ ID NO:32). In some embodiments, the Wnt-binding agent comprises a Fri domain comprising the minimal Fri domain of FZD8 (SEQ ID NO:30).

In some embodiments, the Wnt-binding agent comprises a Fri domain consisting essentially of the Fri domain of FZD1, the Fri domain of FZD2, the Fri domain of FZD3, the Fri domain of FZD4, the Fri domain of FZD5, the Fri domain of FZD6, the Fri domain of FZD7, the Fri domain of FZD8, the Fri domain of FZD9, or the Fri domain of FZD10. In some embodiments, the Wnt-binding agent comprises a Fri domain consisting essentially of the Fri domain of FZD8.

In some embodiments, the Wnt-binding agent comprises a sequence selected from the group consisting of: SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, and SEQ ID NO:33. In some embodiments, the Wnt-binding agent comprises a Fri domain consisting essentially of SEQ ID NO:20. In some embodiments, the Wnt-binding agent comprises a Fri domain consisting essentially of SEQ ID NO:33.

In certain embodiments, the Wnt-binding agent comprises a variant of any one of the aforementioned FZD Fri domain sequences that comprises one or more (e.g., one, two, three, four, five, six, seven, eight, nine, ten, etc.) conservative substitutions and is capable of binding Wnt protein(s).

In certain embodiments, a Wnt-binding agent, such as an agent comprising a Fri domain of a human FZD receptor, further comprises a non-FZD polypeptide. In some embodiments, a FZD soluble receptor may include FZD ECD or Fri domains linked to other non-FZD functional and structural polypeptides including, but not limited to, a human Fc region, protein tags (e.g., myc, FLAG, GST), other endogenous proteins or protein fragments, or any other useful protein sequence including any linker region between a FZD ECD or Fri domain and a second polypeptide. In certain embodiments, the non-FZD polypeptide comprises a human Fc region. The Fc region can be obtained from any of the classes of immunoglobulin, IgG, IgA, IgM, IgD and IgE. In some embodiments, the Fc region is a human IgG1 Fc region. In some embodiments, the Fc region is a human IgG2 Fc region. In some embodiments, the Fc region is a wild-type Fc region. In some embodiments, the Fc region is a mutated Fc region. In some embodiments, the Fc region is truncated at the N-terminal end by 1, 2, 3, 4, 5, 6, 7, 8, 9, or 10 amino acids, (e.g., in the hinge domain). In some embodiments, an amino acid in the hinge domain is changed to hinder undesirable disulfide bond formation. In some embodiments, a cysteine is replaced with a serine to hinder or block undesirable disulfide bond formation. In some embodiments, the Fc region is truncated at the C-terminal end by 1, 2, 3, or more amino acids. In some embodiments, the Fc region is truncated at the C-terminal end by 1 amino acid. In certain embodiments, the non-FZD polypeptide comprises SEQ ID NO:34, SEQ ID NO:35, SEQ ID NO:36, SEQ ID NO:37, or SEQ ID NO:38. In certain embodiments, the non-FZD polypeptide consists essentially of SEQ ID NO:34, SEQ ID NO:35, SEQ ID NO:36, SEQ ID NO:37, or SEQ ID NO:38. In certain embodiments, the non-FZD polypeptide consists essentially of SEQ ID NO:36 or SEQ ID NO:37.

In certain embodiments, a Wnt-binding agent is a fusion protein comprising at least a minimal Fri domain of a FZD receptor and a Fc region. As used herein, a “fusion protein” is a hybrid protein expressed by a nucleic acid molecule comprising nucleotide sequences of at least two genes. In some embodiments, the C-terminus of the first polypeptide is linked to the N-terminus of the immunoglobulin Fc region. In some embodiments, the first polypeptide (e.g., a FZD Fri domain) is directly linked to the Fc region (i.e. without an intervening linker). In some embodiments, the first polypeptide is linked to the Fc region via a linker.

As used herein, the term “linker” refers to a linker inserted between a first polypeptide (e.g., a FZD component) and a second polypeptide (e.g., a Fc region). In some embodiments, the linker is a peptide linker. Linkers should not adversely affect the expression, secretion, or bioactivity of the polypeptide. Linkers should not be antigenic and should not elicit an immune response. Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. Linkers may include, but are not limited to, SerGly, GGSG, GSGS, GGGS, S(GGS)n where n is 1-7, GRA, poly(Gly), poly(Ala), ESGGGGVT (SEQ ID NO:57), LESGGGGVT (SEQ ID NO:58), GRAQVT (SEQ ID NO:59), WRAQVT (SEQ ID NO:60), and ARGRAQVT (SEQ ID NO:61). As used herein, a “linker” is an intervening peptide sequence that does not include amino acid residues from either the C-terminus of the first polypeptide (e.g., a FZD Fri domain) or the N-terminus of the second polypeptide (e.g., the Fc region).

In some embodiments, the Wnt-binding agent comprises a FZD Fri domain, a Fc region, and a linker connecting the FZD Fri domain to the Fc region. In some embodiments, the FZD Fri domain comprises SEQ ID NO:20, SEQ ID NO:30, or SEQ ID NO:33. In some embodiments, the linker comprises ESGGGGVT (SEQ ID NO:57) or LESGGGGVT (SEQ ID NO:58).

In some embodiments, the Wnt-binding agent comprises a first polypeptide comprising SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33; and a second polypeptide comprising SEQ ID NO:34, SEQ ID NO:35, SEQ ID NO:36, SEQ ID NO:37, or SEQ ID NO:38, wherein the first polypeptide is directly linked to the second polypeptide. In some embodiments, the Wnt-binding agent comprises a first polypeptide comprising SEQ ID NO:20 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide comprising SEQ ID NO:20 and a second polypeptide comprising SEQ ID NO:36 or SEQ ID NO:37. In some embodiments, the Wnt-binding agent comprises a first polypeptide consisting essentially of SEQ ID NO:20 and a second polypeptide consisting essentially of SEQ ID NO:36 or SEQ ID NO:37. In some embodiments, the Wnt-binding agent comprises a first polypeptide comprising SEQ ID NO:30 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide comprising SEQ ID NO:30 and a second polypeptide comprising SEQ ID NO:36 or SEQ ID NO:37. In some embodiments, the Wnt-binding agent comprises a first polypeptide comprising SEQ ID NO:33 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide comprising SEQ ID NO:33 and a second polypeptide comprising SEQ ID NO:36, SEQ ID NO:37, or SEQ ID NO:35. In some embodiments, the Wnt-binding agent comprises a first polypeptide consisting essentially of SEQ ID NO:33 and a second polypeptide consisting essentially of SEQ ID NO:36, SEQ ID NO:37, or SEQ ID NO:35.

In some embodiments, the Wnt-binding agent comprises a first polypeptide comprising SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33; and a second polypeptide comprising SEQ ID NO:34, SEQ ID NO:35, SEQ ID NO:36, SEQ ID NO:37, or SEQ ID NO:38, wherein the first polypeptide is connected to the second polypeptide by a linker. In some embodiments, the Wnt-binding agent comprises a first polypeptide comprising SEQ ID NO:20 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide comprising SEQ ID NO:20 and a second polypeptide comprising SEQ ID NO:36 or SEQ ID NO:37. In some embodiments, the Wnt-binding agent comprises a first polypeptide consisting essentially of SEQ ID NO:20 and a second polypeptide consisting essentially of SEQ ID NO:36 or SEQ ID NO:37. In some embodiments, the Wnt-binding agent comprises a first polypeptide comprising SEQ ID NO:30 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide comprising SEQ ID NO:33 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide comprising SEQ ID NO:33 and a second polypeptide comprising SEQ ID NO:36, SEQ ID NO:37, or SEQ ID NO:35. In some embodiments, the Wnt-binding agent comprises a first polypeptide consisting essentially of SEQ ID NO:33 and a second polypeptide consisting essentially of SEQ ID NO:36, SEQ ID NO:37, or SEQ ID NO:35.

In some embodiments, the Wnt-binding agent comprises a first polypeptide that is at least 95% identical to SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33; and a second polypeptide comprising SEQ ID NO:34, SEQ ID NO:35, SEQ ID NO:36, SEQ ID NO:37, or SEQ ID NO:38, wherein the first polypeptide is directly linked to the second polypeptide. In some embodiments, the Wnt-binding agent comprises a first polypeptide that is at least 95% identical to SEQ ID NO:20 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide that is at least 95% identical to SEQ ID NO:30 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide that is at least 95% identical to SEQ ID NO:33 and a second polypeptide comprising SEQ ID NO:34, SEQ ID NO:35, SEQ ID NO:36, SEQ ID NO:37, or SEQ ID NO:38.

In some embodiments, the Wnt-binding agent comprises a first polypeptide that is at least 95% identical to SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33; and a second polypeptide comprising SEQ ID NO:34, SEQ ID NO:35, SEQ ID NO:36, SEQ ID NO:37, or SEQ ID NO:38, wherein the first polypeptide is connected to the second polypeptide by a linker. In some embodiments, the Wnt-binding agent comprises a first polypeptide that is at least 95% identical to SEQ ID NO:20 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide that is at least 95% identical to SEQ ID NO:30 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide that is at least 95% identical to SEQ ID NO:33 and a second polypeptide comprising SEQ ID NO:34, SEQ ID NO:35, SEQ ID NO:36, SEQ ID NO:37, or SEQ ID NO:38.

FZD proteins contain a signal sequence that directs the transport of the proteins. Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides described herein may comprise a mixture of polypeptides with different N-termini. In some embodiments, the N-termini differ in length by 1, 2, 3, 4, 5, 6, 7, 8, 9, 10, or more amino acids. In some embodiments, the N-termini differ in length by 1, 2, 3, 4, or 5 amino acids. In some embodiments, the polypeptide is substantially homogeneous, i.e., the polypeptides have the same N-terminus In some embodiments, the signal sequence of the polypeptide comprises one or more (e.g., one, two, three, four, five, six, seven, eight, nine, ten, etc.) amino acid substitutions and/or deletions. In some embodiments, the signal sequence of the polypeptide comprises amino acid substitutions and/or deletions that allow one cleavage site to be dominant, thereby resulting in a substantially homogeneous polypeptide with one N-terminus.

In some embodiments, the Wnt-binding agent comprises an amino acid sequence selected from the group consisting of: SEQ ID NO:39, SEQ ID NO:40, SEQ ID NO:41, SEQ ID NO:42, SEQ ID NO:43, SEQ ID NO:44, and SEQ ID NO:45.

In certain embodiments, the Wnt-binding agent comprises the sequence of SEQ ID NO:39. In certain embodiments, the agent comprises the sequence of SEQ ID NO:39, comprising one or more (e.g., one, two, three, four, five, six, seven, eight, nine, ten, etc.) conservative substitutions. In certain embodiments, the agent comprises a sequence having at least about 90%, about 95%, or about 98% sequence identity with SEQ ID NO:39. In certain embodiments, the variants of SEQ ID NO:39 maintain the ability to bind one or more human Wnt proteins.

In certain embodiments, the Wnt-binding agent comprises the sequence of SEQ ID NO:40. In some embodiments, the Wnt-binding agent is SEQ ID NO:40. In certain alternative embodiments, the agent comprises the sequence of SEQ ID NO:40, comprising one or more (e.g., one, two, three, four, five, six, seven, eight, nine, ten, etc.) conservative substitutions. In certain embodiments, the agent comprises a sequence having at least about 90%, about 95%, or about 98% sequence identity with SEQ ID NO:40. In certain embodiments, the variants of SEQ ID NO:40 maintain the ability to bind one or more human Wnt proteins.

In certain embodiments, the Wnt-binding agent comprises the sequence of SEQ ID NO:41. In some embodiments, the Wnt-binding agent is SEQ ID NO:41. In certain alternative embodiments, the agent comprises the sequence of SEQ ID NO:41, comprising one or more (e.g., one, two, three, four, five, six, seven, eight, nine, ten, etc.) conservative substitutions. In certain embodiments, the agent comprises a sequence having at least about 90%, about 95%, or about 98% sequence identity with SEQ ID NO:41. In certain embodiments, the variants of SEQ ID NO:41 maintain the ability to bind one or more human Wnt proteins.

In some embodiments, the Wnt-binding agent is OMP-54F28.

In certain embodiments, a Wnt-binding agent is a polypeptide comprising an amino acid sequence selected from the group consisting of: SEQ ID NO:39, SEQ ID NO:40, SEQ ID NO:41, SEQ ID NO:42, SEQ ID NO:43, SEQ ID NO:44, and SEQ ID NO:45. In certain embodiments, the polypeptide comprises an amino acid sequence selected from the group consisting of SEQ ID NO:39, SEQ ID NO:40, and SEQ ID NO:41. In some embodiments, a polypeptide consists essentially of an amino acid sequence selected from the group consisting of: SEQ ID NO:39, SEQ ID NO:40, and SEQ ID NO:41. In certain embodiments, the polypeptide comprises the amino acid sequence of SEQ ID NO:39. In some embodiments, the polypeptide comprises the amino acid sequence of SEQ ID NO:40. In certain embodiments, the polypeptide comprises the amino acid sequence of SEQ ID NO:41. In certain embodiments, the polypeptide comprises the amino acid sequence of SEQ ID NO:42. In certain embodiments, the polypeptide comprises the amino acid sequence of SEQ ID NO:43. In certain embodiments, the polypeptide comprises the amino acid sequence of SEQ ID NO:44. In certain embodiments, the polypeptide comprises the amino acid sequence of SEQ ID NO:45.

In some embodiments, the polypeptide is a substantially purified polypeptide comprising an amino acid sequence selected from the group consisting of SEQ ID NO:39, SEQ ID NO:40, and SEQ ID NO:41. In some embodiments, the polypeptide is a substantially purified polypeptide comprising SEQ ID NO:41. In certain embodiments, the substantially purified polypeptide consists of at least 90% of a polypeptide that has an N-terminal sequence of ASA. In some embodiments, the nascent polypeptide comprises a signal sequence that results in a substantially homogeneous polypeptide product with one N-terminal sequence.

In certain embodiments, a Wnt-binding agent comprises a Fc region of an immunoglobulin. Those skilled in the art will appreciate that some of the binding agents of this invention will comprise fusion proteins in which at least a portion of the Fc region has been deleted or otherwise altered so as to provide desired biochemical characteristics, such as increased cancer cell localization, increased tumor penetration, reduced serum half-life, or increased serum half-life, when compared with a fusion protein of approximately the same immunogenicity comprising a native or unaltered constant region. Modifications to the Fc region may include additions, deletions, or substitutions of one or more amino acids in one or more domains. The modified fusion proteins disclosed herein may comprise alterations or modifications to one or more of the two heavy chain constant domains (CH2 or CH3) or to the hinge region. In other embodiments, the entire CH2 domain may be removed (ΔCH2 constructs). In some embodiments, the omitted constant region domain is replaced by a short amino acid spacer (e.g., 10 aa residues) that provides some of the molecular flexibility typically imparted by the absent constant region domain.

In some embodiments, the modified fusion proteins are engineered to link the CH3 domain directly to the hinge region. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the fusion protein.

In some embodiments, the modified fusion proteins may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete that part of one or more constant region domains that control a specific effector function (e.g., complement Clq binding). Such partial deletions of the constant regions may improve selected characteristics of the binding agent (e.g., serum half-life) while leaving other desirable functions associated with the subject constant region domain intact. Moreover, as alluded to above, the constant regions of the disclosed fusion proteins may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified fusion protein. In certain embodiments, the modified fusion proteins comprise the addition of one or more amino acids to the constant region to enhance desirable characteristics such as decreasing or increasing effector function, or provide for more cytotoxin or carbohydrate attachment sites.

It is known in the art that the constant region mediates several effector functions. For example, binding of the Cl component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an immunoglobulin can bind to a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells, release of inflammatory mediators, placental transfer, and control of immunoglobulin production.

In some embodiments, the modified fusion proteins provide for altered effector functions that, in turn, affect the biological profile of the administered agent. For example, in some embodiments, the deletion or inactivation (through point mutations or other means) of a constant region domain may reduce Fc receptor binding of the circulating modified agent, thereby increasing cancer cell localization and/or tumor penetration. In other embodiments, the constant region modifications increase or reduce the serum half-life of the agent. In some embodiments, the constant region is modified to eliminate disulfide linkages or oligosaccharide moieties.

In certain embodiments, a modified fusion protein does not have one or more effector functions normally associated with an Fc region. In some embodiments, the agent has no antibody-dependent cell-mediated cytotoxicity (ADCC) activity, and/or no complement-dependent cytotoxicity (CDC) activity. In certain embodiments, the agent does not bind to the Fc receptor and/or complement factors. In certain embodiments, the agent has no effector function.

In some embodiments, the Wnt-binding agent (e.g., a soluble receptor) described herein is modified to reduce immunogenicity. In general, immune responses against completely normal human proteins are rare when these proteins are used as therapeutics. However, although many fusion proteins comprise polypeptides sequences that are the same as the sequences found in nature, several therapeutic fusion proteins have been shown to be immunogenic in mammals. In some studies, a fusion protein comprising a linker has been found to be more immunogenic than a fusion protein that does not contain a linker. Accordingly, in some embodiments, the polypeptides of the invention are analyzed by computation methods to predict immunogenicity. In some embodiments, the polypeptides are analyzed for the presence of T-cell and/or B-cell epitopes. If any T-cell or B-cell epitopes are identified and/or predicted, modifications to these regions (e.g., amino acid substitutions) may be made to disrupt or destroy the epitopes. Various algorithms and software that can be used to predict T-cell and/or B-cell epitopes are known in the art. For example, the software programs SYFPEITHI, HLA Bind, PEPVAC, RANKPEP, DiscoTope, ElliPro, and Antibody Epitope Prediction are all publicly available.

In some embodiments, a cell producing any of the Wnt-binding agents (e.g., soluble receptors) or polypeptides described herein is provided. In some embodiments, a composition comprising any of the Wnt-binding agents (e.g., soluble receptors) or polypeptides described herein is provided. In some embodiments, the composition comprises a polypeptide wherein at least 80%, 90%, 95%, 97%, 98%, or 99% of the polypeptide has an N-terminal sequence of ASA. In some embodiments, the composition comprises a polypeptide wherein 100% of the polypeptide has an N-terminal sequence of ASA. In some embodiments, the composition comprises a polypeptide wherein at least 80% of the polypeptide has an N-terminal sequence of ASA. In some embodiments, the composition comprises a polypeptide wherein at least 90% of the polypeptide has an N-terminal sequence of ASA. In some embodiments, the composition comprises a polypeptide wherein at least 95% of the polypeptide has an N-terminal sequence of ASA.

The polypeptides described herein can be recombinant polypeptides, natural polypeptides, or synthetic polypeptides. It will be recognized in the art that some amino acid sequences of the invention can be varied without significant effect on the structure or function of the protein. If such differences in sequence are contemplated, it should be remembered that there will be critical areas on the protein which determine activity. Thus, the invention further includes variations of the polypeptides which show substantial activity or which include regions of FZD proteins, such as the protein portions discussed herein. Such mutants include deletions, insertions, inversions, repeats, and type substitutions.

Of course, the number of amino acid substitutions a skilled artisan would make depends on many factors, including those described above. In certain embodiments, the number of substitutions for any given soluble receptor polypeptide will not be more than 50, 40, 30, 25, 20, 15, 10, 5 or 3.

Fragments or portions of the polypeptides of the present invention can be employed for producing the corresponding full-length polypeptide by peptide synthesis; therefore, the fragments can be employed as intermediates for producing the full-length polypeptides. These fragments or portion of the polypeptides can also be referred to as “protein fragments” or “polypeptide fragments”.

A “protein fragment” of this invention is a portion or all of a protein which is capable of binding to one or more human Wnt proteins or one or more human FZD proteins. In some embodiments, the fragment has a high affinity for one or more human Wnt proteins. In some embodiments, the fragment has a high affinity for one or more human FZD proteins. Some fragments of Wnt-binding agents described herein are protein fragments comprising at least part of the extracellular portion of a FZD protein linked to at least part of a constant region of an immunoglobulin (e.g., a Fc region). The binding affinity of the protein fragment can be in the range of about 10⁻¹¹ to 10⁻¹²M, although the affinity can vary considerably with fragments of different sizes, ranging from 10⁻⁷ to 10⁻¹³M. In some embodiments, the fragment is about 100 to about 200 amino acids in length and comprises a binding domain linked to at least part of a constant region of an immunoglobulin.

In some embodiments, the Wnt pathway inhibitors are polyclonal antibodies. Polyclonal antibodies can be prepared by any known method. In some embodiments, polyclonal antibodies are raised by immunizing an animal (e.g., a rabbit, rat, mouse, goat, donkey) by multiple subcutaneous or intraperitoneal injections of an antigen of interest (e.g., a purified peptide fragment, full-length recombinant protein, or fusion protein). The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood and/or ascites of the immunized animal. The polyclonal antibodies can be purified from serum or ascites according to standard methods in the art including, but not limited to, affinity chromatography, ion-exchange chromatography, gel electrophoresis, and dialysis.

In some embodiments, the Wnt pathway inhibitors are monoclonal antibodies. Monoclonal antibodies can be prepared using hybridoma methods known to one of skill in the art (see e.g., Kohler and Milstein, 1975, Nature, 256:495-497). In some embodiments, using the hybridoma method, a mouse, hamster, or other appropriate host animal, is immunized as described above to elicit from lymphocytes the production of antibodies that will specifically bind the immunizing antigen. In some embodiments, lymphocytes can be immunized in vitro. In some embodiments, the immunizing antigen can be a human protein or a portion thereof. In some embodiments, the immunizing antigen can be a mouse protein or a portion thereof.

Following immunization, lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen may be identified by a variety of methods including, but not limited to, immunoprecipitation, immunoblotting, and in vitro binding assay (e.g., flow cytometry, FACS, ELISA, and radioimmunoassay). The hybridomas can be propagated either in in vitro culture using standard methods (J. W. Goding, 1996, Monoclonal Antibodies: Principles and Practice, 3rd Edition, Academic Press, San Diego, Calif.) or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins. In other embodiments, recombinant monoclonal antibodies, or fragments thereof, can be isolated from phage display libraries.

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted for those regions of, for example, a human antibody to generate a chimeric antibody, or for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In some embodiments, the Wnt pathway inhibitor is a humanized antibody. Typically, humanized antibodies are human immunoglobulins in which amino acid residues of the CDRs are replaced by amino acid residues of a CDR from an immunoglobulin of a non-human species (e.g., mouse, rat, rabbit, hamster, etc.) that have the desired specificity, affinity, and/or binding capability using methods known to one skilled in the art. In some embodiments, Fv framework region amino acid residues of a human immunoglobulin are replaced with corresponding amino acid residues from an antibody of a non-human species that has the desired specificity, affinity, and/or binding capability. In some embodiments, the humanized antibody can be further modified by the substitution of additional amino acid residues either in the Fv framework region and/or within the replaced non-human amino acid residues to refine and optimize antibody specificity, affinity, and/or capability. In general, the humanized antibody will comprise substantially all of at least one, and typically two, variable domain regions containing all, or substantially all, of the CDRs that correspond to the non-human immunoglobulin whereas all, or substantially all, of the framework regions are those of a human immunoglobulin sequence. In some embodiments, the humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject. Methods used to generate humanized antibodies are well known in the art.

In certain embodiments, the Wnt pathway inhibitor is a human antibody. Human antibodies can be directly prepared using various techniques known in the art. In some embodiments, immortalized human B lymphocytes immunized in vitro or isolated from an immunized individual that produces an antibody directed against a target antigen can be generated. In some embodiments, the human antibody can be selected from a phage library, where that phage library expresses human antibodies. Alternatively, phage display technology can be used to produce human antibodies and antibody fragments in vitro, from immunoglobulin variable domain gene repertoires from unimmunized donors. Techniques for the generation and use of antibody phage libraries are well-known in the art. Affinity maturation strategies including, but not limited to, chain shuffling (Marks et al., 1992, Bio/Technology, 10:779-783) and site-directed mutagenesis, are known in the art and may be employed to generate high affinity human antibodies.

In some embodiments, human antibodies can be made in transgenic mice that contain human immunoglobulin loci. These mice are capable, upon immunization, of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

This invention also encompasses bispecific antibodies that specifically recognize at least one human FZD protein or at least one Wnt protein. Bispecific antibodies are capable of specifically recognizing and binding at least two different epitopes. The different epitopes can either be within the same molecule (e.g., two different epitopes on human FZD5) or on different molecules (e.g., one epitope on FZD5 and a different epitope on a second protein). In some embodiments, the bispecific antibodies are monoclonal human or humanized antibodies. In some embodiments, the antibodies can specifically recognize and bind a first antigen target, (e.g., a FZD protein) as well as a second antigen target, such as an effector molecule on a leukocyte (e.g., CD2, CD3, CD28, CD80, or CD86) or a Fc receptor (e.g., CD64, CD32, or CD16) so as to focus cellular defense mechanisms to the cell expressing the first antigen target. In some embodiments, the antibodies can be used to direct cytotoxic agents to cells which express a particular target antigen. These antibodies possess an antigen-binding arm and an arm which binds a cytotoxic agent or a radionuclide chelator, such as EOTUBE, DPTA, DOTA, or TETA.

Bispecific antibodies can be intact antibodies or antibody fragments. Antibodies with more than two valencies are also contemplated. For example, trispecific antibodies can be prepared (Tutt et al., 1991, J. Immunol., 147:60). Thus, in certain embodiments the antibodies are multispecific. Techniques for making bispecific and multispecific antibodies are known by those skilled in the art.

In certain embodiments, the antibodies (or other polypeptides) described herein may be monospecific. For example, in certain embodiments, each of the one or more antigen-binding sites that an antibody contains is capable of binding (or binds) a homologous epitope on different proteins. In certain embodiments, an antigen-binding site of a monospecific antibody described herein is capable of binding (or binds), for example, FZD5 and FZD7 (i.e., the same epitope is found on both FZD5 and FZD7 proteins).

In certain embodiments, the Wnt pathway inhibitor is an antibody fragment comprising an antigen-binding site. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries to allow rapid and effective identification of monoclonal Fab fragments with the desired specificity for a FZD or Wnt protein or derivatives, fragments, analogs or homologs thereof. In some embodiments, antibody fragments are linear antibody fragments. In certain embodiments, antibody fragments are monospecific or bispecific. In certain embodiments, the Wnt pathway inhibitor is a scFv. Various techniques can be used for the production of single-chain antibodies specific to one or more human FZD proteins or one or more human Wnt proteins.

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis). In some embodiments, an antibody is modified to decrease its serum half-life.

Heteroconjugate antibodies are also within the scope of the present invention. Heteroconjugate antibodies are composed of two covalently joined antibodies. Such antibodies have, for example, been proposed to target immune cells to unwanted cells. It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies can comprise any type of variable region that provides for the association of the antibody with the target (i.e., a human FZD protein or a human Wnt protein). In this regard, the variable region may comprise or be derived from any type of mammal that can be induced to mount a humoral response and generate immunoglobulins against the desired tumor-associated antigen. As such, the variable region of the modified antibodies can be, for example, of human, murine, non-human primate (e.g. cynomolgus monkeys, macaques, etc.) or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs will be derived preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the activity of the antigen-binding site.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics such as increased tumor localization and/or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In some embodiments, the modified antibodies will comprise domain deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete the part of one or more constant region domains that control a specific effector function (e.g. complement Clq binding). Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment sites.

It is known in the art that the constant region mediates several effector functions. For example, binding of the Cl component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells, release of inflammatory mediators, placental transfer, and control of immunoglobulin production.

In certain embodiments, the Wnt pathway inhibitors are antibodies that provide for altered effector functions. These altered effector functions may affect the biological profile of the administered antibody. For example, in some embodiments, the deletion or inactivation (through point mutations or other means) of a constant region domain may reduce Fc receptor binding of the circulating modified antibody (e.g., anti-FZD antibody) thereby increasing cancer cell localization and/or tumor penetration. In other embodiments, the constant region modifications increase or reduce the serum half-life of the antibody. In some embodiments, the constant region is modified to eliminate disulfide linkages or oligosaccharide moieties. Modifications to the constant region in accordance with this invention may easily be made using well known biochemical or molecular engineering techniques well within the purview of the skilled artisan.

In certain embodiments, a Wnt pathway inhibitor is an antibody does not have one or more effector functions. For instance, in some embodiments, the antibody has no ADCC activity, and/or no CDC activity. In certain embodiments, the antibody does not bind an Fc receptor, and/or complement factors. In certain embodiments, the antibody has no effector function.

The present invention further embraces variants and equivalents which are substantially homologous to the chimeric, humanized, and human antibodies, or antibody fragments thereof, set forth herein. These can contain, for example, conservative substitution mutations, i.e. the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art and described herein.

In certain embodiments, the antibodies described herein are isolated. In certain embodiments, the antibodies described herein are substantially pure.

In some embodiments of the present invention, the Wnt pathway inhibitors are polypeptides. The polypeptides can be recombinant polypeptides, natural polypeptides, or synthetic polypeptides comprising an antibody, or fragment thereof, that bind at least one human FZD protein or at least one Wnt protein. It will be recognized in the art that some amino acid sequences of the invention can be varied without significant effect on the structure or function of the protein. Thus, the invention further includes variations of the polypeptides which show substantial activity or which include regions of an antibody, or fragment thereof, against a human FZD protein or a Wnt protein. In some embodiments, amino acid sequence variations of FZD-binding polypeptides or Wnt-binding polypeptides include deletions, insertions, inversions, repeats, and/or other types of substitutions.

The polypeptides, analogs and variants thereof, can be further modified to contain additional chemical moieties not normally part of the polypeptide. The derivatized moieties can improve the solubility, the biological half-life, and/or absorption of the polypeptide. The moieties can also reduce or eliminate any undesirable side effects of the polypeptides and variants. An overview for chemical moieties can be found in Remington: The Science and Practice of Pharmacy, 22st Edition, 2012, Pharmaceutical Press, London.

The isolated polypeptides that can be used in the methods described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding binding agents (e.g., antibodies or soluble receptors), or fragments thereof, against a human FZD protein or a Wnt protein. For example, recombinant expression vectors can be replicable DNA constructs which have synthetic or cDNA-derived DNA fragments encoding a polypeptide chain of a FZD-binding agent, a Wnt-binding agent, an anti-FZD antibody or fragment thereof, an anti-Wnt antibody or fragment thereof, or a FZD-Fc soluble receptor operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of a FZD-binding or Wnt-binding agent (or a protein to use as an antigen) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are well-known in the art. Additional information regarding methods of protein production, including antibody production, can be found, e.g., in U.S. Patent Publication No. 2008/0187954, U.S. Pat. Nos. 6,413,746 and 6,660,501, and International Patent Publication No. WO 2004/009823.

Various mammalian culture systems are used to express recombinant polypeptides. Expression of recombinant proteins in mammalian cells may be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art (see, e.g., Luckow and Summers, 1988, Bio/Technology, 6:47).

Thus, the present invention provides cells comprising the FZD-binding agents or the Wnt-binding agents described herein. In some embodiments, the cells produce the binding agents (e.g., antibodies or soluble receptors) described herein. In certain embodiments, the cells produce an antibody. In certain embodiments, the cells produce antibody OMP-18R5. In some embodiments, the cells produce a soluble receptor. In some embodiments, the cells produce a FZD-Fc soluble receptor. In some embodiments, the cells produce a FZD8-Fc soluble receptor. In some embodiments, the cells produce FZD8-Fc soluble receptor 54F28.

The proteins produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Isolated proteins can also be physically characterized using such techniques as proteolysis, mass spectrometry (MS), nuclear magnetic resonance (NMR), high performance liquid chromatography (HPLC), and x-ray crystallography.

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a binding agent. Some or all of the foregoing purification steps, in various combinations, can also be employed to provide a homogeneous recombinant protein.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005.

In certain embodiments, the Wnt-binding agent or the FZD-binding agent is a polypeptide that is not an antibody. A variety of methods for identifying and producing non-antibody polypeptides that bind with high affinity to a protein target are known in the art. See, e.g., Skerra, 2007, Curr. Opin. Biotechnol., 18:295-304; Hosse et al., 2006, Protein Science, 15:14-27; Gill et al., 2006, Curr. Opin. Biotechnol., 17:653-658; Nygren, 2008, FEBS J., 275:2668-76; and Skerra, 2008, FEBS J., 275:2677-83. In certain embodiments, phage display technology may be used to produce and/or identify a FZD-binding or Wnt-binding polypeptide. In certain embodiments, the polypeptide comprises a protein scaffold of a type selected from the group consisting of protein A, protein G, a lipocalin, a fibronectin domain, an ankyrin consensus repeat domain, and thioredoxin.

In certain embodiments, the binding agents can be used in any one of a number of conjugated (i.e. an immunoconjugate or radioconjugate) or non-conjugated forms. In certain embodiments, antibodies can be used in a non-conjugated form to harness the subject's natural defense mechanisms including complement-dependent cytotoxicity and antibody dependent cellular toxicity to eliminate the malignant or cancer cells.

In some embodiments, the binding agent is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated antibody. A variety of radionuclides are available for the production of radioconjugated antibodies including, but not limited to, ⁹⁰ Y, ¹²⁵I,¹³¹I, ¹²³I, ¹¹¹In, ¹³¹In, ¹⁰⁵Rh, ¹⁵³Sm, ⁶⁷Cu, ⁶⁷Ga, ¹⁶⁶Ho,¹⁷⁷Lu, ¹⁸⁶Re, ¹⁸⁸Re and ²¹²Bi. In some embodiments, conjugates of an antibody and one or more small molecule toxins, such as a calicheamicin, maytansinoids, a trichothene, and CC1065, and the derivatives of these toxins that have toxin activity, can be produced. In certain embodiments, conjugates of an antibody and a cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In certain embodiments, the Wnt pathway inhibitor (e.g., antibody or soluble receptor) is an antagonist of at least one Wnt protein (i.e., 1, 2, 3, 4, 5, 6, 7, 8, 9, or 10 Wnt proteins). In certain embodiments, the Wnt pathway inhibitor inhibits activity of the Wnt protein(s) to which it binds. In certain embodiments, the Wnt pathway inhibitor inhibits at least about 10%, at least about 20%, at least about 30%, at least about 50%, at least about 75%, at least about 90%, or about 100% of the activity of the human Wnt protein(s) to which it binds.

In certain embodiments, the Wnt pathway inhibitor (e.g., antibody or soluble receptor) inhibits binding of at least one human Wnt to an appropriate receptor. In certain embodiments, the Wnt pathway inhibitor inhibits binding of at least one human Wnt protein to one or more human FZD proteins. In some embodiments, the at least one Wnt protein is selected from the group consisting of: Wnt1, Wnt2, Wnt2b/13, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In some embodiments, the one or more human FZD proteins are selected from the group consisting of: FZD1, FZD2, FZD3, FZD4, FZD5, FZD6, FZD7, FZD8, FZD9, and FZD 10. In certain embodiments, the Wnt pathway inhibitor inhibits binding of one or more Wnt proteins to FZD1, FZD2, FZD4, FZD5, FZD7, and/or FZD8. In certain embodiments, the Wnt pathway inhibitor inhibits binding of one or more Wnt proteins to FZD8. In certain embodiments, the inhibition of binding of a particular Wnt to a FZD protein by a Wnt pathway inhibitor is at least about 10%, at least about 25%, at least about 50%, at least about 75%, at least about 90%, or at least about 95%. In certain embodiments, an agent that inhibits binding of a Wnt to a FZD protein, also inhibits Wnt pathway signaling. In certain embodiments, a Wnt pathway inhibitor that inhibits human Wnt pathway signaling is an antibody. In certain embodiments, a Wnt pathway inhibitor that inhibits human Wnt pathway signaling is a FZD-Fc soluble receptor. In certain embodiments, a Wnt pathway inhibitor that inhibits human Wnt pathway signaling is a FZD8-Fc soluble receptor. In certain embodiments, a Wnt pathway inhibitor that inhibits human Wnt pathway signaling is soluble receptor 54F28.

In certain embodiments, the Wnt pathway inhibitors (e.g., antibody or soluble receptor) described herein are antagonists of at least one human Wnt protein and inhibit Wnt activity. In certain embodiments, the Wnt pathway inhibitor inhibits Wnt activity by at least about 10%, at least about 20%, at least about 30%, at least about 50%, at least about 75%, at least about 90%, or about 100%. In some embodiments, the Wnt pathway inhibitor inhibits activity of one, two, three, four, five or more Wnt proteins. In some embodiments, the Wnt pathway inhibitor inhibits activity of at least one human Wnt protein selected from the group consisting of: Wnt1, Wnt2, Wnt2b, Wnt3, Wnt3 a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In some embodiments, the Wnt-binding agent binds at least one Wnt protein selected from the group consisting of Wnt1, Wnt2, Wnt2b, Wnt3, Wnt3a, Wnt7a, Wnt7b, Wnt8a, Wnt8b, Wnt10a, and Wnt10b. In certain embodiments, the at least one Wnt protein is selected from the group consisting of Wnt1, Wnt2, Wnt2b, Wnt3, Wnt3a, Wnt8a, Wnt8b, Wnt10a, and Wnt10b. In certain embodiments, a Wnt pathway inhibitor that inhibits human Wnt activity is an antibody. In certain embodiments, a Wnt pathway inhibitor that inhibits human Wnt activity is a FZD-Fc soluble receptor. In certain embodiments, a Wnt pathway inhibitor that inhibits human Wnt activity is a FZD8-Fc soluble receptor. In certain embodiments, a Wnt pathway inhibitor that inhibits human Wnt activity is soluble receptor 54F28.

In certain embodiments, the Wnt pathway inhibitor described herein is an antagonist of at least one human FZD protein and inhibits FZD activity. In certain embodiments, the Wnt pathway inhibitor inhibits FZD activity by at least about 10%, at least about 20%, at least about 30%, at least about 50%, at least about 75%, at least about 90%, or about 100%. In some embodiments, the Wnt pathway inhibitor inhibits activity of one, two, three, four, five or more FZD proteins. In some embodiments, the Wnt pathway inhibitor inhibits activity of at least one human FZD protein selected from the group consisting of: FZD1, FZD2, FZD3, FZD4, FZD5, FZD6, FZD7, FZD8, FZD9, and FZD 10. In certain embodiments, the Wnt pathway inhibitor inhibits activity of FZD 1, FZD2, FZD4, FZD5, FZD7, and/or FZD8. In certain embodiments, the Wnt pathway inhibitor inhibits activity of FZD8. In some embodiments, the Wnt pathway inhibitor is an anti-FZD antibody. In certain embodiments, the Wnt pathway inhibitor is anti-FZD antibody OMP-18R5.

In certain embodiments, the Wnt pathway inhibitor described herein is an antagonist of at least one human Wnt protein and inhibits Wnt signaling. In certain embodiments, the Wnt pathway inhibitor inhibits Wnt signaling by at least about 10%, at least about 20%, at least about 30%, at least about 50%, at least about 75%, at least about 90%, or about 100%. In some embodiments, the Wnt pathway inhibitor inhibits signaling by one, two, three, four, five or more Wnt proteins. In some embodiments, the Wnt pathway inhibitor inhibits signaling of at least one Wnt protein selected from the group consisting of Wnt1, Wnt2, Wnt2b, Wnt3, Wnt3a, Wnt7a, Wnt7b, Wnt8a, Wnt8b, Wnt10a, and Wnt10b. In certain embodiments, a Wnt pathway inhibitor that inhibits Wnt signaling is an antibody. In certain embodiments, a Wnt pathway inhibitor that inhibits Wnt signaling is a soluble receptor. In certain embodiments, a Wnt pathway inhibitor that inhibits Wnt signaling is a FZD-Fc soluble receptor. In certain embodiments, a Wnt pathway inhibitor that inhibits Wnt signaling is a FZD8-Fc soluble receptor. In certain embodiments, a Wnt pathway inhibitor that inhibits Wnt signaling is soluble receptor 54F28.

In certain embodiments, a Wnt pathway inhibitor described herein is an antagonist of β-catenin signaling. In certain embodiments, the Wnt pathway inhibitor inhibits β-catenin signaling by at least about 10%, at least about 20%, at least about 30%, at least about 50%, at least about 75%, at least about 90%, or about 100%. In certain embodiments, a Wnt pathway inhibitor that inhibits β-catenin signaling is an antibody. In certain embodiments, a Wnt pathway inhibitor that inhibits β-catenin signaling is an anti-FZD antibody. In certain embodiments, a Wnt pathway inhibitor that inhibits β-catenin signaling is antibody OMP-18R5. In certain embodiments, a Wnt pathway inhibitor that inhibits β-catenin signaling is a soluble receptor. In certain embodiments, a Wnt pathway inhibitor that inhibits β-catenin signaling is a FZD-Fc soluble receptor. In certain embodiments, a Wnt pathway inhibitor that inhibits β-catenin signaling is a FZD8-Fc soluble receptor.

In certain embodiments, the Wnt pathway inhibitor described herein inhibits binding of at least one Wnt protein to a receptor. In certain embodiments, the Wnt pathway inhibitor inhibits binding of at least one human Wnt protein to one or more of its receptors. In some embodiments, the Wnt pathway inhibitor inhibits binding of at least one Wnt protein to at least one FZD protein. In some embodiments, the Wnt-binding agent inhibits binding of at least one Wnt protein to FZD 1, FZD2, FZD3, FZD4, FDZ5, FDZ6, FDZ7, FDZ8, FDZ9, and/or FZD10. In certain embodiments, the inhibition of binding of at least one Wnt to at least one FZD protein is at least about 10%, at least about 25%, at least about 50%, at least about 75%, at least about 90%, or at least about 95%. In certain embodiments, a Wnt pathway inhibitor that inhibits binding of at least one Wnt to at least one FZD protein further inhibits Wnt pathway signaling and/or β-catenin signaling. In certain embodiments, a Wnt pathway inhibitor that inhibits binding of at least one human Wnt to at least one FZD protein is an antibody. In certain embodiments, a Wnt pathway inhibitor that inhibits binding of at least one human Wnt to at least one FZD protein is an anti-FZD antibody. In certain embodiments, a Wnt pathway inhibitor that inhibits binding of at least one human Wnt to at least one FZD protein is antibody OMP-18R5. In certain embodiments, a Wnt pathway inhibitor that inhibits binding of at least one human Wnt to at least one FZD protein is a soluble receptor. In certain embodiments, a Wnt pathway inhibitor that inhibits binding of at least one human Wnt to at least one FZD protein is a FZD-Fc soluble receptor. In certain embodiments, a Wnt pathway inhibitor that inhibits binding of at least one human Wnt to at least one FZD protein is a FZD8-Fc soluble receptor. In certain embodiments, a Wnt pathway inhibitor that inhibits binding of at least one human Wnt to at least one FZD protein is FZD8-Fc soluble receptor 54F28.

In certain embodiments, the Wnt pathway inhibitor described herein blocks binding of at least one Wnt to a receptor. In certain embodiments, the Wnt pathway inhibitor blocks binding of at least one human Wnt protein to one or more of its receptors. In some embodiments, the Wnt pathway inhibitor blocks binding of at least one Wnt to at least one FZD protein. In some embodiments, the Wnt pathway inhibitor blocks binding of at least one Wnt protein to FZD1, FZD2, FZD3, FZD4, FDZ5, FDZ6, FDZ7, FDZ8, FDZ9, and/or FZD10. In certain embodiments, the blocking of binding of at least one Wnt to at least one FZD protein is at least about 10%, at least about 25%, at least about 50%, at least about 75%, at least about 90%, or at least about 95%. In certain embodiments, a Wnt pathway inhibitor that blocks binding of at least one Wnt protein to at least one FZD protein further inhibits Wnt pathway signaling and/or β-catenin signaling. In certain embodiments, a Wnt pathway inhibitor that blocks binding of at least one human Wnt to at least one FZD protein is an antibody. In certain embodiments, a Wnt pathway inhibitor that blocks binding of at least one human Wnt to at least one FZD protein is an anti-FZD antibody. In certain embodiments, a Wnt pathway inhibitor that blocks binding of at least one human Wnt to at least one FZD protein is antibody OMP-18R5. In certain embodiments, a Wnt pathway inhibitor that blocks binding of at least one human Wnt to at least one FZD protein is a soluble receptor. In certain embodiments, a Wnt pathway inhibitor that blocks binding of at least one human Wnt to at least one FZD protein is a FZD-Fc soluble receptor. In certain embodiments, a Wnt pathway inhibitor that blocks binding of at least one human Wnt to at least one FZD protein is a FZD8-Fc soluble receptor. In certain embodiments, a Wnt pathway inhibitor that blocks binding of at least one human Wnt to at least one FZD protein is soluble receptor 54F28.

In certain embodiments, the Wnt pathway inhibitor described herein inhibits Wnt pathway signaling. It is understood that a Wnt pathway inhibitor that inhibits Wnt pathway signaling may, in certain embodiments, inhibit signaling by one or more receptors in the Wnt signaling pathway but not necessarily inhibit signaling by all receptors. In certain alternative embodiments, Wnt pathway signaling by all human receptors may be inhibited. In certain embodiments, Wnt pathway signaling by one or more receptors selected from the group consisting of FZD 1, FZD2, FZD3, FZD4, FDZ5, FDZ6, FDZ7, FDZ8, FDZ9, and FZD10 is inhibited. In certain embodiments, the inhibition of Wnt pathway signaling by a Wnt pathway inhibitor is a reduction in the level of Wnt pathway signaling of at least about 10%, at least about 25%, at least about 50%, at least about 75%, at least about 90%, or at least about 95%. In some embodiments, a Wnt pathway inhibitor that inhibits Wnt pathway signaling is an antibody. In some embodiments, a Wnt pathway inhibitor that inhibits Wnt pathway signaling is an anti-FZD antibody. In some embodiments, a Wnt pathway inhibitor that inhibits Wnt pathway signaling is antibody OMP-18R5. In some embodiments, a Wnt pathway inhibitor that inhibits Wnt pathway signaling is a soluble receptor. In some embodiments, a Wnt pathway inhibitor that inhibits Wnt pathway signaling is a FZD-Fc soluble receptor. In some embodiments, a Wnt pathway inhibitor that inhibits Wnt pathway signaling is a FZD8-Fc soluble receptor. In some embodiments, a Wnt pathway inhibitor that inhibits Wnt pathway signaling is soluble receptor 54F28.

In certain embodiments, the Wnt pathway inhibitor described herein inhibits activation of β-catenin. It is understood that a Wnt pathway inhibitor that inhibits activation of β-catenin may, in certain embodiments, inhibit activation of β-catenin by one or more receptors, but not necessarily inhibit activation of β-catenin by all receptors. In certain alternative embodiments, activation of β-catenin by all human receptors may be inhibited. In certain embodiments, activation of β-catenin by one or more receptors selected from the group consisting of FZD1, FZD2, FZD3, FZD4, FDZ5, FDZ6, FDZ7, FDZ8, FDZ9, and FZD10 is inhibited. In certain embodiments, the inhibition of activation of β-catenin by a Wnt-binding agent is a reduction in the level of activation of β-catenin of at least about 10%, at least about 25%, at least about 50%, at least about 75%, at least about 90%, or at least about 95%. In some embodiments, a Wnt pathway inhibitor that inhibits activation of β-catenin is an antibody. In some embodiments, a Wnt pathway inhibitor that inhibits activation of β-catenin is an anti-FZD antibody. In some embodiments, a Wnt pathway inhibitor that inhibits activation of β-catenin is antibody OMP-18R5. In some embodiments, a Wnt pathway inhibitor that inhibits activation of β-catenin is a soluble receptor. In some embodiments, a Wnt pathway inhibitor that inhibits activation of β-catenin is a FZD-Fc soluble receptor. In some embodiments, a Wnt pathway inhibitor that inhibits activation of β-catenin is a FZD8-Fc soluble receptor. In some embodiments, a Wnt pathway inhibitor that inhibits activation of β-catenin is soluble receptor 54F28.

In vivo and in vitro assays for determining whether a Wnt pathway inhibitor inhibits β-catenin signaling are known in the art. For example, cell-based, luciferase reporter assays utilizing a TCF/Luc reporter vector containing multiple copies of the TCF-binding domain upstream of a firefly luciferase reporter gene may be used to measure β-catenin signaling levels in vitro (Gazit et al., 1999, Oncogene, 18; 5959-66; TOPflash, Millipore, Billerica MA). The level of β-catenin signaling in the presence of one or more Wnt proteins (e.g., Wnt(s) expressed by transfected cells or provided by Wnt-conditioned media) in the presence of a binding agent is compared to the level of signaling without the binding agent present. In addition to the TCF/Luc reporter assay, the effect of a binding agent (or candidate agent) on β-catenin signaling may be measured in vitro or in vivo by measuring the effect of the agent on the level of expression of β-catenin-regulated genes, such as c-myc (He et al., 1998, Science, 281:1509-12), cyclin D1 (Tetsu et al., 1999, Nature, 398:422-6), and/or fibronectin (Gradl et al. 1999, Mol. Cell Biol., 19:5576-87). In certain embodiments, the effect of a binding agent on β-catenin signaling may also be assessed by measuring the effect of the agent on the phosphorylation state of Dishevelled-1, Dishevelled-2, Dishevelled-3, LRP5, LRP6, and/or β-catenin.

In certain embodiments, a Wnt pathway inhibitor has one or more of the following effects: inhibit proliferation of tumor cells, inhibit tumor growth, reduce the frequency of cancer stem cells in a tumor, reduce the tumorigenicity of a tumor, reduce the tumorigenicity of a tumor by reducing the frequency of cancer stem cells in the tumor, trigger cell death of tumor cells, induce cells in a tumor to differentiate, differentiate tumorigenic cells to a non-tumorigenic state, induce expression of differentiation markers in the tumor cells, prevent metastasis of tumor cells, or decrease survival of tumor cells.

In certain embodiments, a Wnt pathway inhibitor is capable of inhibiting tumor growth. In certain embodiments, a Wnt pathway inhibitor is capable of inhibiting tumor growth in vivo (e.g., in a xenograft mouse model, and/or in a human having cancer). In some embodiments, the tumor is a tumor selected from the group consisting of colorectal tumor, colon tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In certain embodiments, the tumor is melanoma. In certain embodiments, the tumor is a colorectal tumor. In certain embodiments, the tumor is a pancreatic tumor. In certain embodiments, the tumor is a breast tumor. In certain embodiments, the tumor is a Wnt-dependent tumor.

In certain embodiments, a Wnt pathway inhibitor is capable of reducing the tumorigenicity of a tumor. In certain embodiments, a Wnt pathway inhibitor is capable of reducing the tumorigenicity of a tumor comprising cancer stem cells in an animal model, such as a mouse xenograft model. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the number or frequency of cancer stem cells is determined by limiting dilution assay using an animal model. Additional examples and guidance regarding the use of limiting dilution assays to determine a reduction in the number or frequency of cancer stem cells in a tumor can be found, e.g., in International Publication No. WO 2008/042236, and U.S. Patent Publication Nos. 2008/0064049 and 2008/0178305.

In certain embodiments, the Wnt pathway inhibitors described herein are active in vivo for at least 1 hour, at least about 2 hours, at least about 5 hours, at least about 10 hours, at least about 24 hours, at least about 2 days, at least about 3 days, at least about 1 week, or at least about 2 weeks. In certain embodiments, the Wnt pathway inhibitor is an IgG (e.g., IgG1 or IgG2) antibody that is active in vivo for at least 1 hour, at least about 2 hours, at least about 5 hours, at least about 10 hours, at least about 24 hours, at least about 2 days, at least about 3 days, at least about 1 week, or at least about 2 weeks. In certain embodiments, the Wnt pathway inhibitor is a fusion protein that is active in vivo for at least 1 hour, at least about 2 hours, at least about 5 hours, at least about 10 hours, at least about 24 hours, at least about 2 days, at least about 3 days, at least about 1 week, or at least about 2 weeks.

In certain embodiments, the Wnt pathway inhibitors described herein have a circulating half-life in mice, cynomolgus monkeys, or humans of at least about 5 hours, at least about 10 hours, at least about 24 hours, at least about 2 days, at least about 3 days, at least about 1 week, or at least about 2 weeks. In certain embodiments, the Wnt pathway inhibitor is an IgG (e.g., IgG1 or IgG2) antibody that has a circulating half-life in mice, cynomolgus monkeys, or humans of at least about 5 hours, at least about 10 hours, at least about 24 hours, at least about 2 days, at least about 3 days, at least about 1 week, or at least about 2 weeks. In certain embodiments, the Wnt pathway inhibitor is a fusion protein that has a circulating half-life in mice, cynomolgus monkeys, or humans of at least about 5 hours, at least about 10 hours, at least about 24 hours, at least about 2 days, at least about 3 days, at least about 1 week, or at least about 2 weeks. Methods of increasing (or decreasing) the half-life of agents such as polypeptides and antibodies are known in the art. For example, known methods of increasing the circulating half-life of IgG antibodies include the introduction of mutations in the Fc region which increase the pH-dependent binding of the antibody to the neonatal Fc receptor (FcRn) at pH 6.0 (see, e.g., U.S. Patent Publication Nos. 2005/0276799, 2007/0148164, and 2007/0122403). Known methods of increasing the circulating half-life of antibody fragments lacking the Fc region include such techniques as PEGylation.

### IV. Kits

Kits for practicing the methods of the invention are further provided. By “kit” is intended any manufacture (e.g., a package or a container) comprising at least one reagent, e.g., an antibody, a nucleic acid probe, etc. for specifically detecting the expression of at least one biomarker of the invention. The kit may be promoted, distributed, and/or sold as a unit for performing the methods of the present invention. Additionally, the kits may contain a package insert describing the kit and including instructional material for its use.

In some embodiments, a kit comprises reagents for practicing the methods of the invention using microarray technology. In some embodiments, a kit comprises reagents for practicing the methods of the invention using qPCR assays. Positive and/or negative controls may be included in the kits to validate the activity and correct usage of reagents employed in accordance with the invention. Controls may include samples known to be either positive or negative for the presence of the biomarker of interest, or other samples comprising the biomarkers of interest. The design and use of controls is standard and well within the routine capabilities of those in the art.

In some embodiments, a kit comprises polynucleotides selected from the group consisting of SEQ ID NOs:62-79. In some embodiments, a kit comprises (a) a forward primer of SEQ ID NO:62, a reverse primer of SEQ ID NO:63, and a probe comprising SEQ ID NO:64; (b) a forward primer of SEQ ID NO:65, a reverse primer of SEQ ID NO:66, and a probe comprising SEQ ID NO:67; (c) a forward primer of SEQ ID NO:68, a reverse primer of SEQ ID NO:69, and a probe comprising SEQ ID NO:70; (d) a forward primer of SEQ ID NO:71, a reverse primer of SEQ ID NO:72, and a probe comprising SEQ ID NO:73; (e) a forward primer of SEQ ID NO:74, a reverse primer of SEQ ID NO:75, and a probe comprising SEQ ID NO:76; and (f) a forward primer of SEQ ID NO:77, a reverse primer of SEQ ID NO:78, and a probe comprising SEQ ID NO:79.

It will be further appreciated that any or all steps in the methods of the invention could be implemented by personnel or, alternatively, performed in an automated fashion. Thus, the steps of sample preparation, detection of biomarker expression, etc. may be automated.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, may be practiced without departing from the scope of the present disclosure.

### EXAMPLES

### Example 1

Identification of Tumors Responsive to Treatment with a Combination of OMP-18R5 and Taxol

The breast tumor xenograft models OMP-B34, OMP-B39, OMP-B44, OMP-B59, OMP-B60, UM-T01, UM-T03, and UM-PE13 were established at OncoMed Pharmaceuticals or the University of Michigan from minimally passaged, patient-derived tumor specimens. Six- to 8-week-old NOD/SCID mice were subcutaneously injected with 2-4×10⁴ cells of OMP-B34, OMP-B39, OMP-B44, OMP-B59, OMP-B60, UM-T01, UM-T03, or UM-PE13 tumors. Tumors were allowed to grow until they reached an average volume of 100 to 150mm³ Tumor-bearing mice were randomized into four groups (n=10 per group) and treated with control antibody 1B711 (15 mg/kg), anti-FZD antibody OMP-18R5 (15 mg/kg), taxol (10 mg/kg), or OMP-18R5 (15 mg/kg) in combination with taxol (10 mg/kg). Treatment with antibodies and/or taxol was administered on a weekly basis. Tumor growth was monitored and tumor volumes were measured with electronic calipers at the indicated time points. Data are expressed as mean ±S.E.M.

To determine if a tumor was responsive to anti-FZD antibody OMP-18R5, single agent tumor volume data was compared with the control while combination treatment with OMP-18R5 and taxol was compared with taxol as a single agent. For this study a “responder” tumor was defined as a tumor showing significantly greater tumor growth inhibition with the combination of OMP-18R5 and taxol as compared to tumor growth inhibition with taxol as single agent.

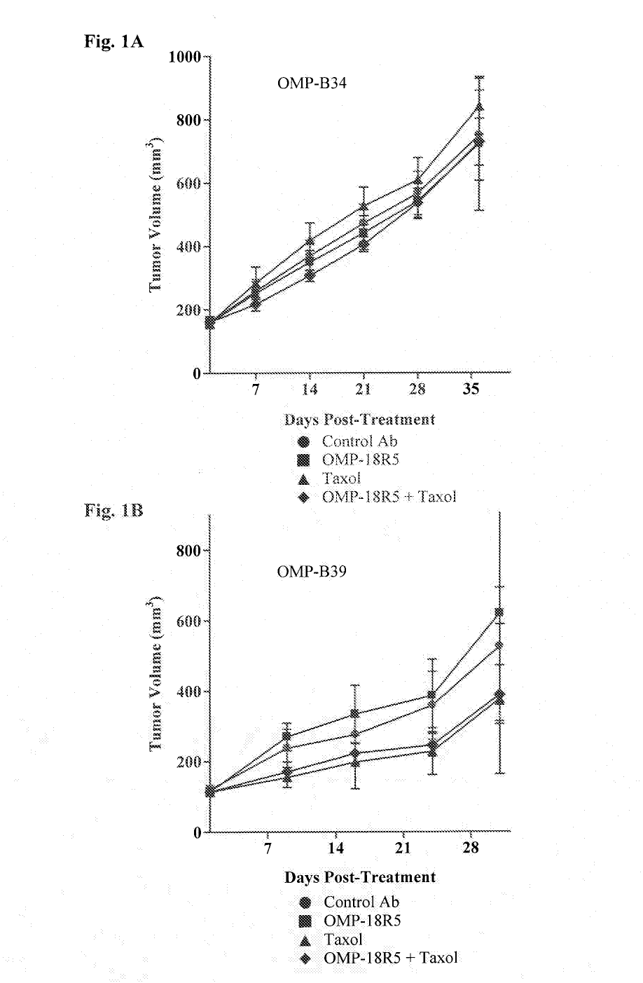

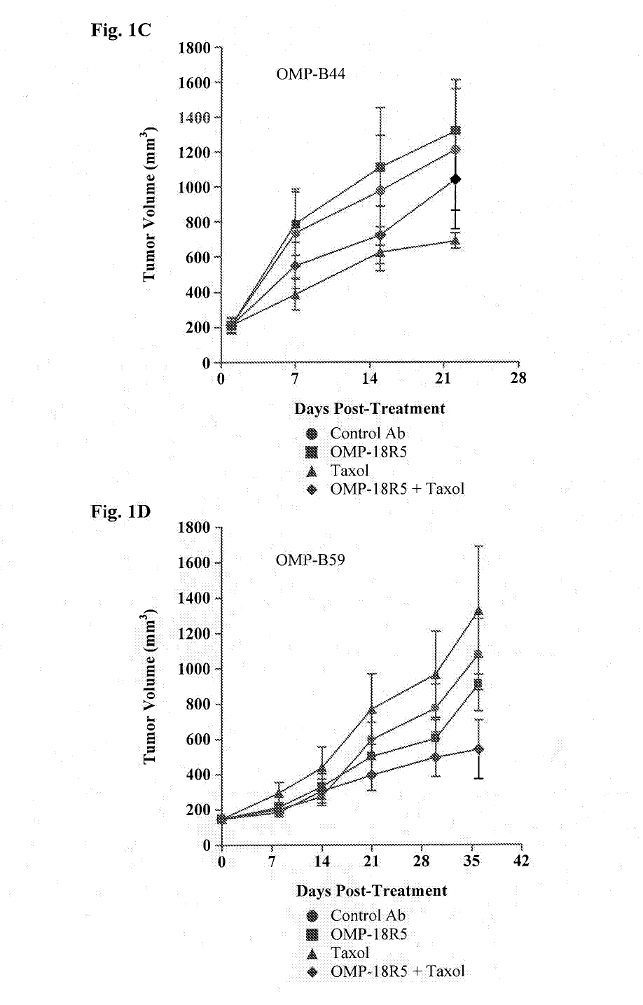

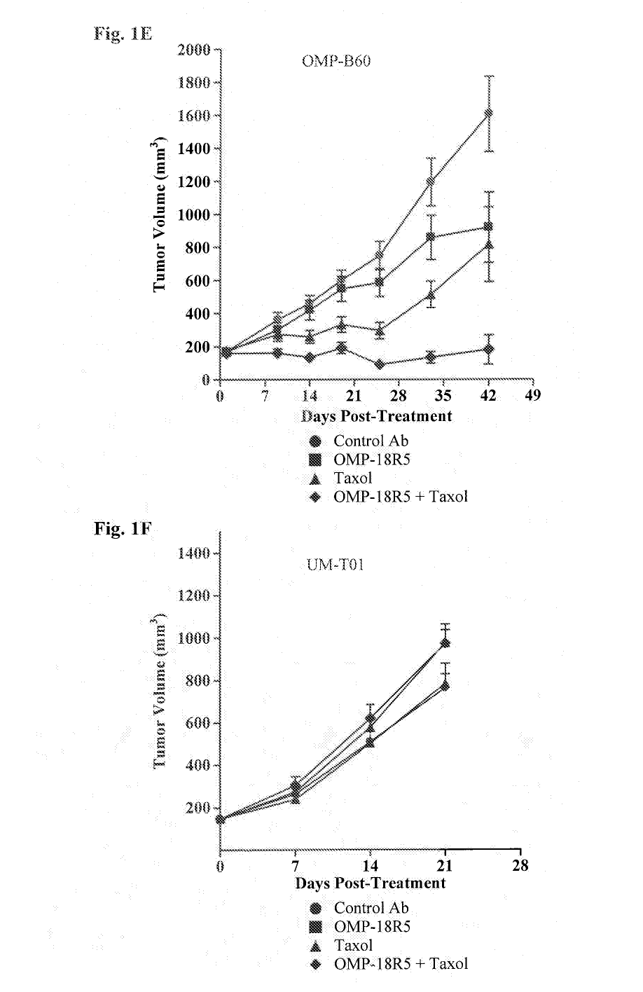

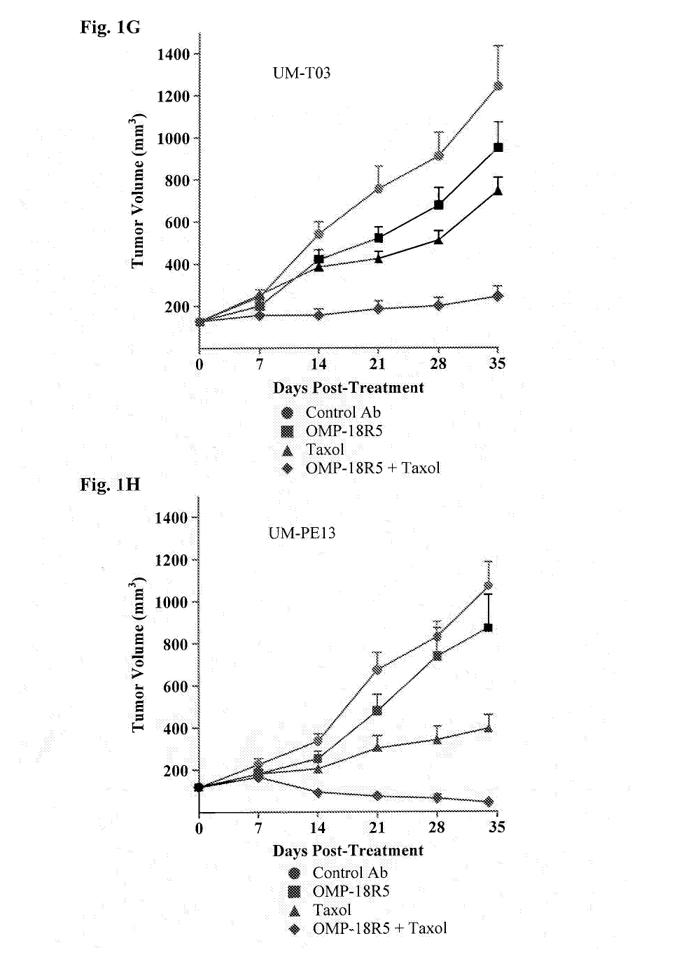

The results for each xenograft model are shown in FIGS. 1A-H. T-tests were conducted at each time point. Multiple comparisons used 2-way repeated measurement ANOVA followed by Bonferroni corrections. The t-tests and 2-way repeated measurement ANOVA were performed using GraphPad Prism5 (GraphPad Software Inc.). The tumors OMP-B59, OMP-B60, UM-T03, and UM-PE13 were shown to be responders, while tumors OMP-B34, OMP-B39, OMP-B44, and UM-T01 were shown to be non-responders. The results are summarized in Table 1.

TABLE 1

| Tumor | Tumor Subtype | Classification |
|---|---|---|
| OMP-B34 | TNBC | Non-Responder |
| OMP-B39 | TNBC | Non-Responder |
| OMP-B44 | TNBC | Non-Responder |
| OMP-B59 | TNBC | Responder |
| OMP-B60 | TNBC | Responder |
| UM-T01 | TNBC | Non-Responder |
| UM-T03 | ER+PR+HER2+ | Responder |
| UM-PE13 | TNBC | Responder |

### Example 2

### Identification of Predictive Biomarkers

Microarray analyses were performed on untreated breast tumors OMP-B34, OMP-B39, OMP-B44 which did not respond to treatment with a combination of OMP-18R5 and taxol, (“non-responders”), and UM-T01 and untreated tumors OMP-B59, OMP-B60, UM-T03, and UM-PE13 which did respond to treatment with a combination of OMP-18R5 and taxol (“responders”). RNA was isolated from each tumor using a RNeasy Fibrous Tissue Mini Kit (Qiagen, Valencia CA) with DNAse treatment following the manufacturer's instructions. Samples were stored at -80° C. RNA was visualized on an Agilent 2100 Bioanalyzer and integrity was confirmed by the presence of intact 28S and 18S ribosomal peaks. All RNA samples had 260/280 ratios>1.8. Total RNA isolated from each tumor was amplified using the Ovation RNA Amplification System V2 (NuGEN, San Carlos, Calif.). Amplified, anti-sense single stranded-cDNA was fragmented and biotinylated using the FL-Ovation cDNA Biotin Module V2 (NuGEN). The quality of the cDNA and the fragmented cDNA was assessed by a spectrophotometer and a Bioanalyzer before hybridization to the array. The processed RNA was hybridized to Affymetrix HG-U133 plus 2.0 microarrays (Affymetrix, Santa Clara, Calif.) as outlined in the manufacturer's technical manuals. After hybridization, the microarrays were washed, scanned, and analyzed. Microarray data were processed to probe set level data by using GeneChip-RMA (Wu et al., 2004, J. Amer. Stat. Assn., 99:909-917). Probe sets that were likely to cross-hybridize with murine markers were removed. To summarize the data to gene level and make sure the probe set with the strongest signals were chosen, maximum expression was used across all probe sets mapping to one gene. Genes with low expression (<5 on log2 scale) or near-zero variance (<0.01) were removed. Genes were standardized to N(0,1) by subtracting the log2 scale expression from the mean and dividing by the standard deviation of each gene.

Analyses were performed using genes from several signaling pathways including canonical, planar cell polarity, Wnt/Ca+2, Wnt signaling negative regulation, cell fate, tissue polarity, cell growth and proliferation, cell migration, cell cycle, and cellular homeostasis (see Table 2).

TABLE 2

| Gene Symbol | Protein Name |
|---|---|
| AES | Amino-terminal enhancer of split |
| APC | Adenomatous polyposis coli protein |
| AXIN1 | Axin-1 |
| BCL9 | B-cell CLL/lymphoma 9 protein |
| BTRC | F-box/WD repeat-containing protein 1A |
| CCND1 | G1/S-specific cyclin-D1 |
| CCND2 | G1/S-specific cyclin-D2 |
| CCND3 | G1/S-specific cyclin-D3 |
| CSNK1A1 | Casein kinase I isoform alpha |
| CSNK1D | Casein kinase I isoform delta |
| CSNK1G1 | Casein kinase I isoform gamma-1 |
| CSNK2A1 | Casein kinase II subunit alpha |
| CTBP1 | C-terminal-binding protein 1 |
| CTBP2 | C-terminal-binding protein 2 |
| CTNNB1 | Catenin beta-1 |
| CTNNBIP1 | Beta-catenin-interacting protein 1 |
| CXXC4 | CXXC-type zinc finger protein 4 |
| DAAM1 | Disheveled-associated activator of morphogenesis 1 |
| DIXDC1 | Dixin |
| DKK1 | Dickkopf-related protein 1 |
| DVL1 | Segment polarity protein disheveled homolog DVL-1 |
| DVL2 | Segment polarity protein disheveled homolog DVL-2 |
| EP300 | Histone acetyltransferase p300 |
| FBXW11 | F-box/WD repeat-containing protein 11 |
| FBXW2 | F-box/WD repeat-containing protein 2 |
| FBXW4 | F-box/WD repeat-containing protein 4 |
| FGF4 | Fibroblast growth factor 4 |
| FOSL1 | Fos-related antigen 1 |
| FOXN1 | Forkhead box protein N1 |
| FRAT1 | Proto-oncogene FRAT1 |
| FRZB | Secreted frizzled-related protein 3 |
| FSHB | Follitropin subunit beta |
| FZD1 | Frizzled-1 |
| FZD2 | Frizzled-2 |
| FZD3 | Frizzled-3 |
| FZD4 | Frizzled-4 |
| FZD5 | Frizzled-5 |
| FZD6 | Frizzled-6 |
| FZD7 | Frizzled-7 |
| FZD8 | Frizzled-8 |
| GSK3A | Glycogen synthase kinase-3 alpha |
| GSK3B | Glycogen synthase kinase-4 alpha |
| JUN | Transcription factor AP-1 |
| KREMEN1 | Kremen protein 1 |
| LEF1 | Lymphoid enhancer-binding factor 1 |
| LRP5 | Low-density lipoprotein receptor-related protein 5 |
| LRP6 | Low-density lipoprotein receptor-related protein 6 |
| MYC | Myc proto-oncogene protein |
| NKD1 | Protein naked cuticle homolog |
| NLK | Serine/threonine-protein kinase NLK |
| PITX2 | Pituitary homeobox 2 |
| PORCN | Protein-cysteine N-palmitoyl transferase porcupine |
| PPP2CA | Serine/threonine-protein phosphatase 2A catalytic |
| | subunit alpha isoform |
| PPP2R1A | Serine/threonine-protein phosphatase 2A 65 kDa |
| | regulatory subunit A alpha isoform |
| PYGO1 | Pygopus homolog 1 |
| RHOU | Rho-related GTP-binding protein RhoU |
| SENP2 | Sentrin-specific protease 2 |
| SFRP1 | Secreted frizzled-related protein 1 |
| SFRP4 | Secreted frizzled-related protein 4 |
| SLC9A3R1 | Na(+)/H(+) exchange regulatory cofactor NHE-RF1 |
| SOX17 | Transcription factor SOX-17 |
| T | Brachyury protein |
| TCF7 | Transcription factor 7 |
| TCF7L1 | Transcription factor 7-like 1 |
| TLE1 | Transducin-like enhancer protein 1 |
| TLE2 | Transducin-like enhancer protein 2 |
| WIF1 | Wnt inhibitory factor 1 |
| WISP1 | WNT1-inducible signaling pathway protein 1 |
| WNT1 | Proto-oncogene Wnt-1 |
| WNT2 | Protein Wnt-2 |
| WNT2B | Protein Wnt-2B |
| WNT3 | Protein Wnt-3 |
| WNT3A | Protein Wnt-3a |
| WNT4 | Protein Wnt-4 |
| WNT5A | Protein Wnt-5a |
| WNT5B | Protein Wnt-5b |
| WNT6 | Protein Wnt-6 |
| WNT7A | Protein Wnt-7a |
| WNT7B | Protein Wnt-7b |
| WNT8A | Protein Wnt-8a |
| WNT9A | Protein Wnt-9a |
| WNT10A | Protein Wnt-10a |
| WNT11 | Protein Wnt-11 |
| WNT16 | Protein Wnt-16 |

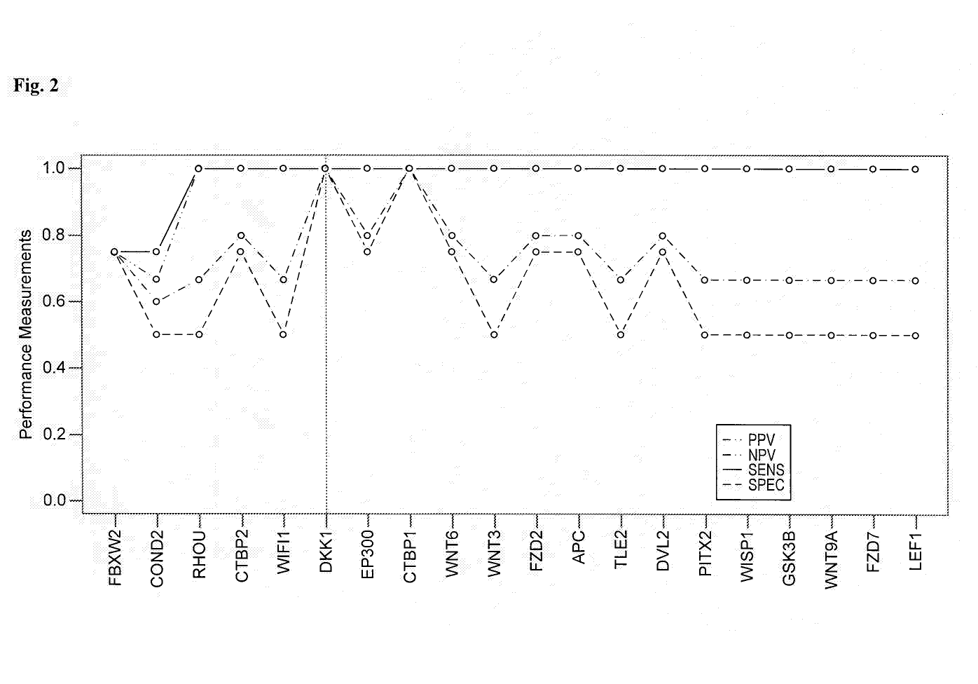

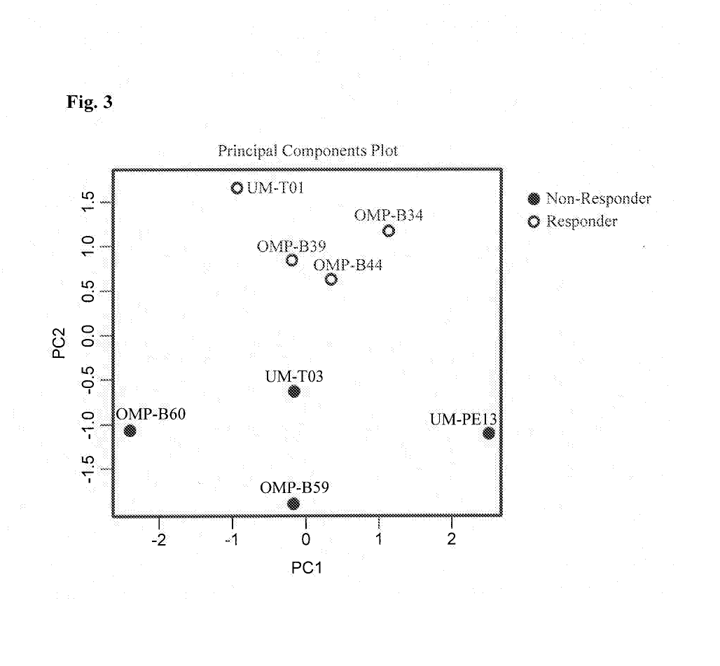

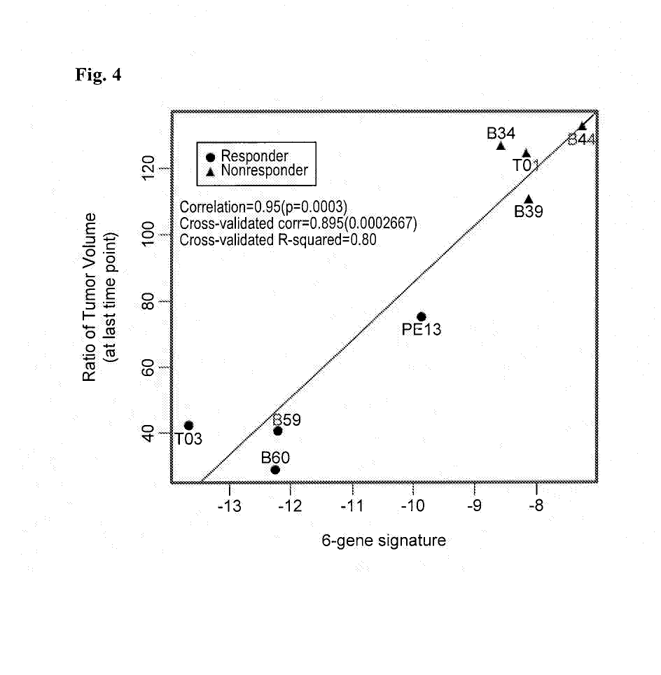

Support Vector Machines-Recursive Feature Elimination (SVM-RFE) methods (Guyon et al, 2002, Machine Learning, 46:389-422) were used to identify genes that could distinguish between the responder and non-responder tumors and Support Vector Machine (SVM) methods (Cortes and Vapnik, 1995, Machine Learning, 20:273-297) were used for classification. A leave-one-out cross-validation (LOOCV) method was used to select the number of genes and also to measure positive predictive value (PPV), negative predictive value (NPV), sensitivity, and specificity of the models. A biomarker signature comprising FBXW2, CCND2, RHOU, CTBP2, WIF1, and DKK1 achieved the best performance with PPV=NPV=sensitivity=specificity=100% using the 8 breast tumors (see FIG. 2). As shown in FIG. 3, principal component analysis (PCA) illustrated that the 6-gene biomarker signature resulted in a near perfect separation of the 8 breast tumors. In addition, strong correlation was observed between the 6-gene biomarker signature and the ratio of tumor volume (RTV) from the in vivo experiments described in Example 1 (correlation=0.95, p-value=0.0003; cross-validated correlation=0.89, p-value=0.00027; FIG. 4).

Decision values were determined from the SVM model based on the training data. For the 6-gene biomarker signature, decision values can be calculated by a weighted sum of the standardized expression of the 6 genes: 0.4560427*FBXW2+0.3378467*CCND2−0.4809354*RHOU+0.409029*CTBP2+0.3291529*WIF1+02926374*DKK1+0.04662682. A positive decision value indicated a tumor predicted to be a responder while a negative decision value indicated a tumor predicted to be a non-responder. In addition, classification probabilities can be obtained by fitting a logistic regression on the decision values. Tumors associated with probabilities higher than 0.5 would be predicted to be a responder while tumors with probabilities lower than 0.5 would be predicted to be a non-responder.

### Example 3

### In Vivo Validation of Predictive Biomarkers

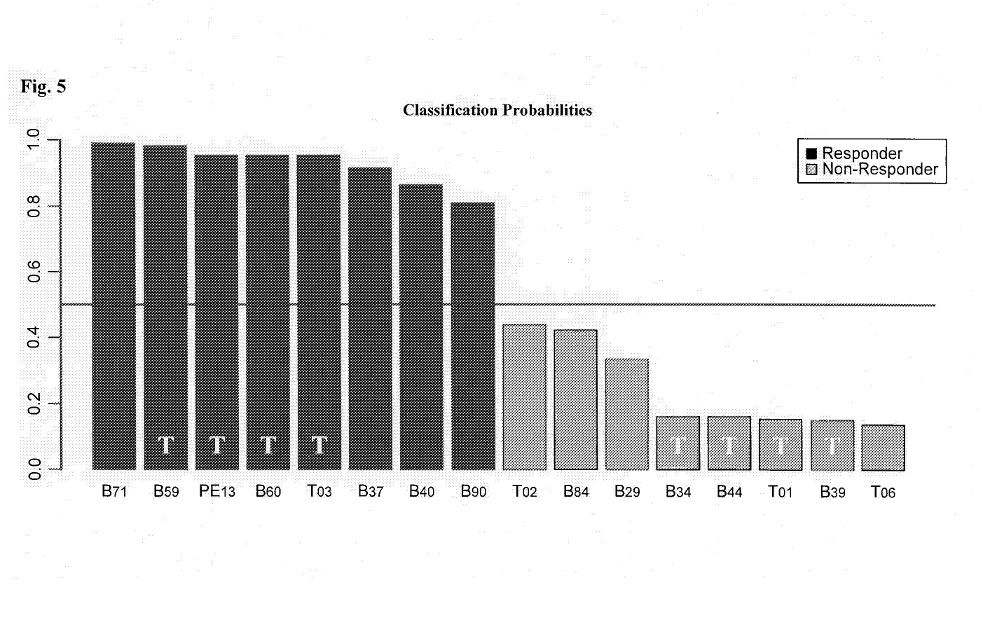

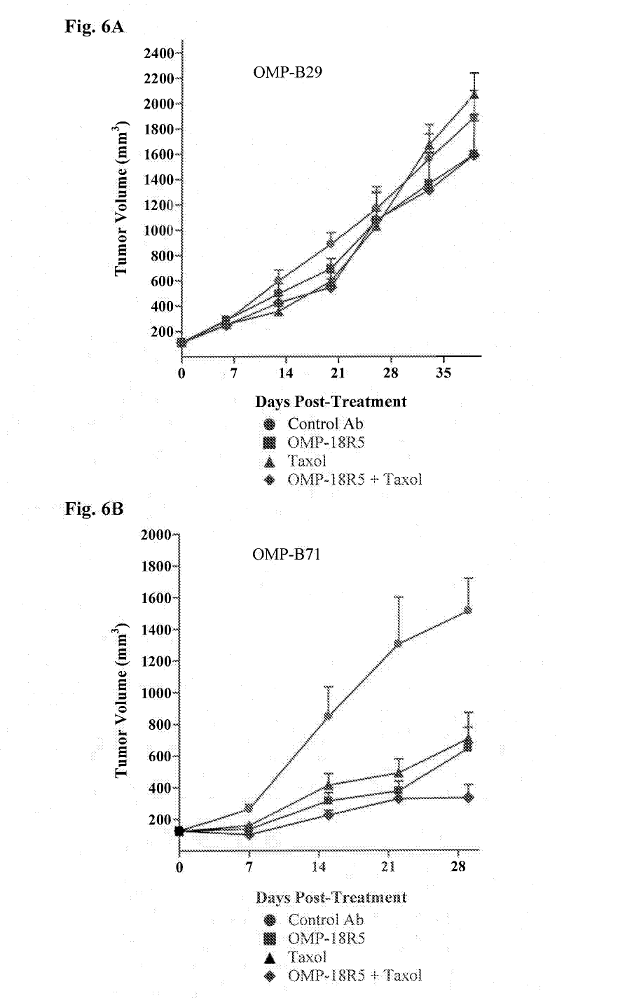

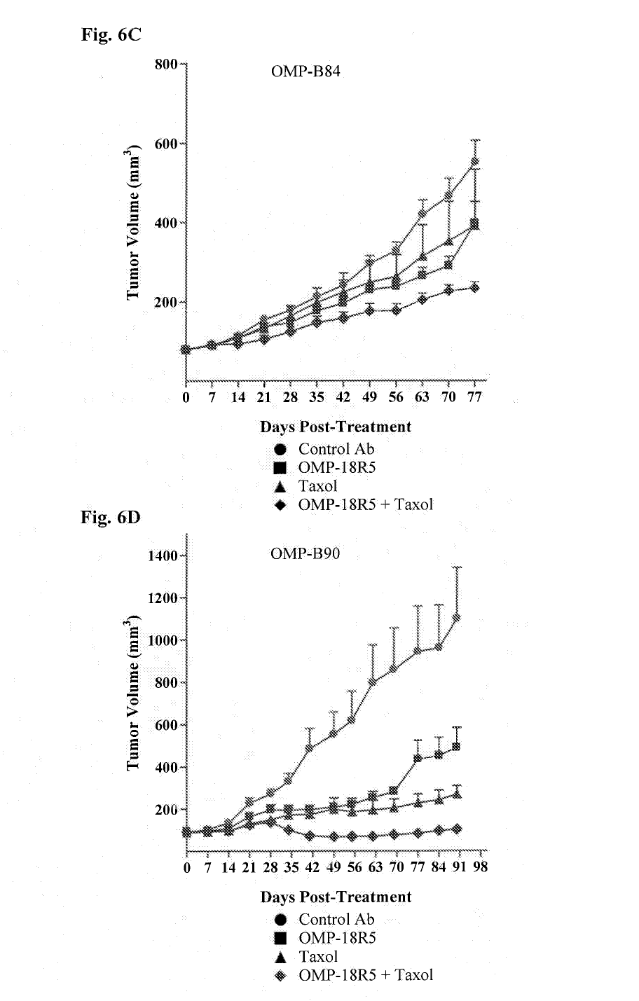

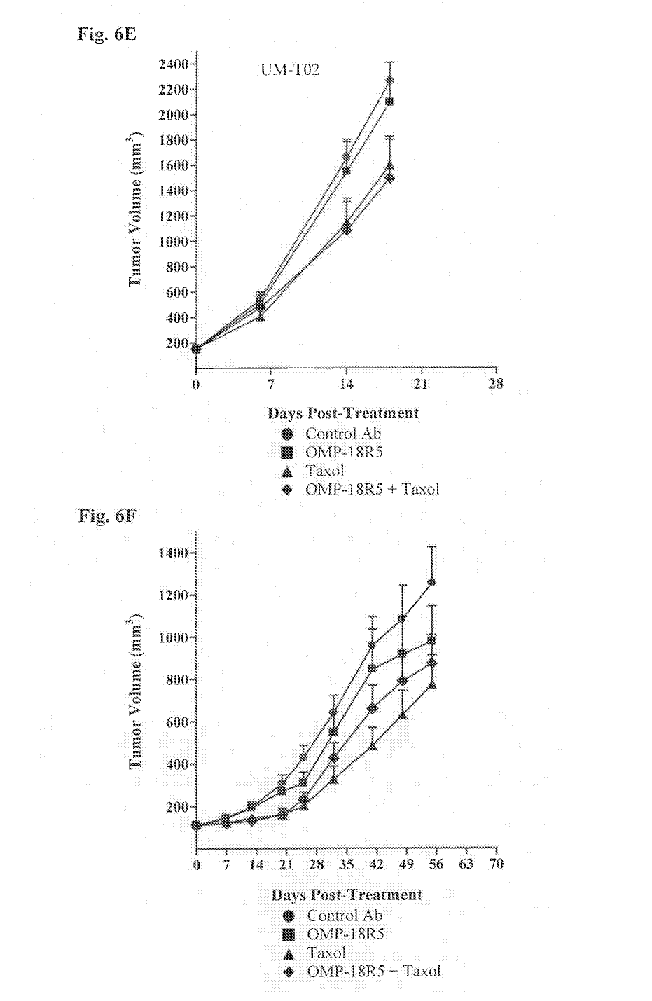

Six additional breast cancer tumors were selected from the OncoMed Tumor Bank and microarray analyses were performed as described in Example 1. The six breast cancer tumors were OMP-B29, OMP-B71, OMP-B84, OMP-B90, UM-T02, and UM-T06. As described herein, classification probability analysis was used with the 6-gene biomarker signature to predict the response of each of these tumors to treatment with anti-FZD antibody OMP-18R5 in combination with taxol (see FIG. 5). In parallel the six tumors were evaluated in in vivo xenograft models as described in Example 1 (see FIGS. 6A-F). As described in Example 1 a “responder” in the in vivo models is a tumor showing significantly greater tumor growth inhibition with the combination of OMP-18R5 and taxol as compared to tumor growth inhibition with taxol as single agent. The predictions based on classification probabilities were compared to the results of the in vivo xenograft models. The results are summarized in Table 3.

TABLE 3

| | | Classi- | | | |
|---|---|---|---|---|---|
| | | fication | | | |
| | | Proba- | Decision | | In vivo |
| Tumor | Tumor subtype | bility | Value | Prediction | Response |
| OMP-B29 | ER+PR+HER2− | 0.3344 | −0.5928 | Non- | Non- |
| | | | | responder | responder |
| OMP-B71 | ER+PR+HER2− | 0.9897 | 1.6789 | Responder | Responder |
| OMP-B84 | ER+PR+HER2− | 0.4324 | −0.4002 | Non- | Non- |
| | | | | responder | responder |
| OMP-B90 | TNBC | 0.8152 | 0.492 | Responder | Responder |
| UM-T02 | TNBC | 0.4387 | −0.3972 | Non- | Non- |
| | | | | responder | responder |
| UM-T06 | ER+PR+HER2− | 0.1385 | −1.0778 | Non- | Non- |
| | | | | responder | responder |

As shown in Table 3, the response of each of the six breast cancer tumors was accurately predicted by the 6-gene biomarker signature using the decision values and the classification probabilities.

### Example 4

### Prevalence Estimation of the 6-Gene Biomarker Signature

Prevalence of a biomarker signature can be defined as the proportion of a population predicted to be a responder based upon the biomarker signature. The prevalence of the 6-gene biomarker signature in HER2 negative (HER2−) and triple negative breast cancer (TNBC) populations was estimated by applying the 6-gene biomarker signature to three publicly available breast cancer microarray data sets. The Cremoux2001 dataset was compiled from Affymetrix U133p1us2 microarrays with 226 patients, including 145 HER2− and 81 HER2+, where 51 TNBC were included within the HER2− group. The Wang2011 dataset was compiled from Affymetrix U133p1us2 microarrays with 115 patients, including 79 HER2− and 36 HER2+, where 28 TNBC were included within the HER2− group. The Prat2010 dataset was compiled from Agilent Human 1A microarrays with 333 patients, including 215 HER2− and 118 HER2+, where 57 TNBC were included within the HER2− group. Pre-processing of the public data included downloading the data, extracting the probe sets mapping to the six genes, and collapsing the probe sets to the six genes. Gene level expression data was further standardized by subtracting the mean and dividing by the standard deviation of each gene in the public data. The SVM model built upon the training data was used to classify the public data. Classification probabilities were obtained and the proportion of predicted responders (probability>0.5) was calculated based on the 6-gene biomarker signature.

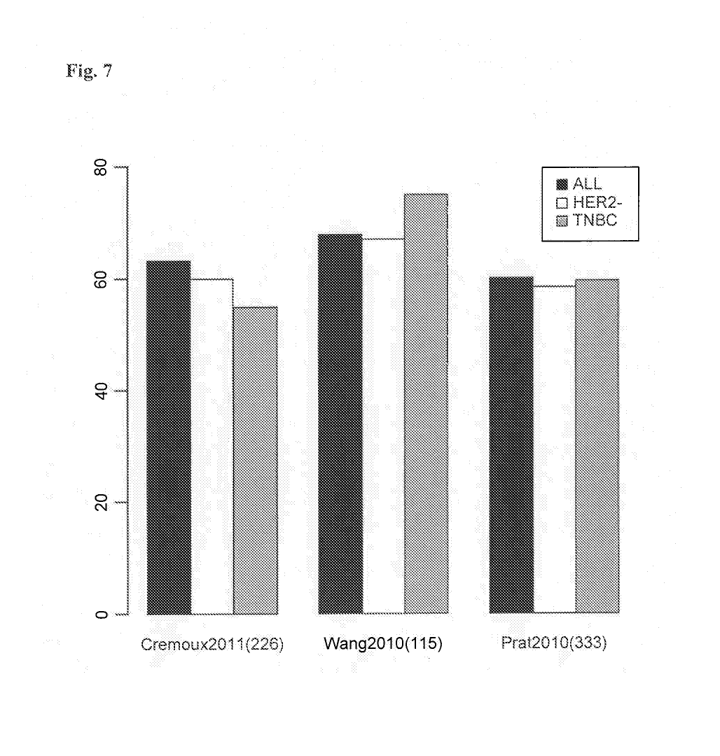

As shown in FIG. 7, the predicted prevalence of the 6-gene biomarker signature within the 3 datasets was very similar (approximately 60%). This prediction would suggest that there is a large population of breast cancer patients that would be responsive to therapy with the anti-FZD antibody OMP-18R5 in combination with taxol.

### Example 5

qPCR Assays for 6-Gene Biomarker Signature

qPCR assays were developed to determine the expression levels of FBXW2, CCND2, RHOU, CTBP2, WIF1, and DKK1 in a tumor sample. Primers and probes were designed using publicly available mRNA sequences. The primers and probes were generated and used in optimization and validation tests using human fresh frozen (FF) and formalin-fixed paraffin-embedded (FFPE) human tissue samples. The specific primers and probes are listed in Table 4 (all sequences in 5′ to 3′ direction). Four reference genes were used for normalization including TOP1 (topoisomerase 1), GUSB (beta-glucuronidase), SDHA (succinate dehydrogenase), and PUM1 (pumilio homolog 1).

TABLE 4

| | Primer/ | | SEQ ID |
|---|---|---|---|
| Gene | Probe | Sequence | NO |
| CCND2 | Forward | GCTGTCTCTG | SEQ ID |
| | Primer | ATCCGCAAGC | NO: 62 |
| | Reverse | GACGGTGGGTA | SEQ ID |
| | Primer | CATGGCAAAC | NO: 63 |
| | Probe | CCTTCATTGCTCT | SEQ ID |
| | | GTGTGCCACCGAC | NO: 64 |
| CTBP2 | Forward | ATCCGTGGG | SEQ ID |
| | Primer | GAGACGCTG | NO: 65 |
| | Reverse | CTCGAACTGC | SEQ ID |
| | Primer | AACCGCCTG | NO: 66 |
| | Probe | CCCGTGCGACCA | SEQ ID |
| | | AAGCCAATGAGG | NO: 67 |
| DKK1 | Forward | GACCATTGACAA | SEQ ID |
| | Primer | CTACCAGCCGTA | NO: 68 |
| | Reverse | TGGGACTAGCG | SEQ ID |
| | Primer | CAGTACTCATC | NO: 69 |
| | Probe | TGCCGCACTCC | SEQ ID |
| | | TCGTCCTCTG | NO: 70 |
| FBXW2 | Forward | GCCAGTTATGATA | SEQ ID |
| | Primer | TTCTCAGGGTCA | NO: 71 |
| | Reverse | AGCAGGGCAAAG | SEQ ID |
| | Primer | ATATCTCCAAA | NO: 72 |
| | Probe | AGACTCCTGAGATA | SEQ ID |
| | | GCAAACTTGGCCT | NO: 73 |
| RHOU1 | Forward | CCCACCGAGTA | SEQ ID |
| | Primer | CATCCCTACTG | NO: 74 |
| | Reverse | CAGTGTCACAGA | SEQ ID |
| | Primer | GTTGGAGTCTCA | NO: 75 |
| | Probe | CGCCCATCCAC | SEQ ID |
| | | AGACACCACCG | NO: 76 |
| WIF1 | Forward | GTTCCAAAGGTT | SEQ ID |
| | Primer | ACCAGGGAGAC | NO: 77 |
| | Reverse | GTTGGGTTCAT | SEQ ID |
| | Primer | GGCAGGTTCC | NO: 78 |
| | Probe | CCAGGCTCGCAGA | SEQ ID |
| | | CAGGCTTTGAAC | NO: 79 |

qPCR was performed on total RNA obtained from 18 xenograft breast tumors. Tumor specimens were harvested and immediately snap frozen and stored at −80° C. prior to RNA isolation. Total RNA was extracted using the RNeasy Fibrous Mini Kit (Qiagen, Valencia Calif., PN#74704) with TissueLyzer homogenization and DNase I treatment according to the manufacturer's protocol. RNAs were visualized on a Bioanalyzer 2100 (Agilent, Santa Clara, Calif.) and verified to be intact with RIN values>6.0. All RNAs had A260/A280 ratios>1.8.

qPCR was performed in a two-step manner. First, cDNA was synthesized from total RNA using random hexamers as described in Applied Biosystems User Bulletin 2. TaqMan Universal PCR Master Mix (Applied Biosystems, Foster City, Calif. Cat #4304437 and 4326708) was used in subsequent qPCR reactions according to the manufacturer's protocol. Quantities of gene expression were determined using a Ct (cycle threshold) method from triplicate reactions. Cycle threshold is generally considered to be the number of cycles required for a signal to cross the detection threshold. Ct levels are inversely proportional to the amount of target nucleic acid in a sample. Ct of the six genes are normalized using the Ct levels of the four reference genes. Normalized Ct of the 6-gene signature for the 18 xenograft samples is shown in Table 5.

TABLE 5

| | FBXW2 | CCND2 | RHOU | CTBP2 | WIF1 | DKK1 |
|---|---|---|---|---|---|---|
| OMP-B84 | 0.8425 | 12.5125 | 4.6775 | 1.0775 | 16.4025 | 5.2575 |
| OMP-B71 | 0.98375 | 14.52375 | 6.46875 | 0.08875 | 4.56875 | 1.14375 |
| OMP-B59 | 0.83875 | 2.67375 | 6.43875 | −0.6012 | 4.90375 | 10.7888 |
| OMP-B86 | 2.4725 | 11.5125 | 2.5425 | 1.3275 | −0.8825 | 1.1125 |
| OMP-B39 | 1.03 | 12.54 | 1.44 | 2.225 | 2.045 | 6.365 |
| OMP-B90 | 1.175 | 6.955 | 6.87 | 1.535 | 17.535 | 10.87 |
| p1 | | | | | | |
| OMP-B94 | 1.67375 | 1.52875 | 5.95375 | 1.56875 | 9.34875 | 4.37875 |
| OMP-B40 | 1.03 | 16.455 | 6.775 | 0.73 | 16.455 | 14.985 |
| OMP-B29 | 1.445 | 13.63 | 6.425 | 0.695 | 13.63 | 4.185 |
| OMP-B60 | 1.6725 | 14.7775 | 6.9825 | 0.4775 | −0.8025 | 8.6775 |
| OMP-B90 | 0.75875 | 14.18375 | 5.78875 | 0.13875 | 15.54375 | 10.7388 |
| p2 | | | | | | |
| UM-T06 | 1.19875 | 11.51875 | 4.27875 | 1.34375 | 8.87375 | 6.26375 |
| OMP-B44 | 1.61 | 11.765 | 4.755 | 0.505 | 7.225 | 9.61 |
| UM-T02 | 2.255 | 13.215 | 4.195 | 1.075 | 16.125 | 4.225 |
| UM-T 3 | 1.67625 | 12.58625 | 5.83625 | 0.20125 | 16.21625 | 4.17125 |
| OMP-B34 | 0.08625 | 0.58625 | 6.21125 | 0.53125 | 9.02125 | 0.06125 |
| UM-PE13 | 0.925 | 15.185 | 4.055 | −0.7 | 15.185 | 6.62 |
| UM-T01 | 2.20375 | 15.11375 | 6.44375 | −0.1062 | 15.11375 | 15.1138 |

Decision values can be calculated by a weighted sum of the normalized expression of the 6 genes from data generated from the qPCR assays. These decision values are different than the decision values generated from the analysis based on microarray data, however the predictive capabilities of the two models are very similar.

It is understood that the examples and embodiments described herein are for illustrative purposes only and that various modifications or changes in light thereof will be suggested to persons skilled in the art and are to be included within the spirit and purview of this application.

All publications, patents, and patent applications cited herein are hereby incorporated by reference in their entirety for all purposes to the same extent as if each individual publication, patent or patent application were specifically and individually indicated to be so incorporated by reference.

Following are the sequences disclosed in the application:

OMP-18R5 Heavy chain CDR1
(SEQ ID NO: 1)
GFTFSHYTLS
OMP-18R5 Heavy chain CDR2
(SEQ ID NO: 2)
VISGDGSYTYYADSVKG
OMP-18R5 Heavy chain CDR3
(SEQ ID NO: 3)
NFIKYVFAN
OMP-18R5 Light chain CDR1
(SEQ ID NO: 4)
SGDNIGSFYVH
OMP-18R5 Light chain CDR2
(SEQ ID NO: 5)
DKSNRPSG
OMP-18R5 Light chain CDR3
(SEQ ID NO: 6)
QSYANTLSL
OMP-18R5 Heavy chain variable region amino acid sequence
(SEQ ID NO: 7)
EVQLVESGGGLVQPGGSLRLSCAASGFTFSHYTLSWVRQAPGKGLEWVSVISGDGSYTYY
ADSVKGRFTISSDNSKNTLYLQMNSLRAEDTAVYYCARNFIKYVFANWGQGTLVTVSS
OMP-18R5 Light chain variable region amino acid sequence
(SEQ ID NO: 8)
DIELTQPPSVSVAPGQTARISCSGDNIGSFYVHWYQQKPGQAPVLVIYDKSNRPSGIPER
FSGSNSGNTATLTISGTQAEDEADYYCQSYANTLSLVFGGGTKLTVLG
OMP-18R5 Heavy chain amino acid sequence with predicted signal
sequence underlined
(SEQ ID NO: 9)
MKHLWFFLLLVAAPRWVLSEVQLVESGGGLVQPGGSLRLSCAASGFTFSHYTLSWVRQAP
GKGLEWVSVISGDGSYTYYADSVKGRFTISSDNSKNTLYLQMNSLRAEDTAVYYCARNFI
KYVFANWGQGTLVTVSSASTKGPSVFPLAPCSRSTSESTAALGCLVKDYFPEPVTVSWNS
GALTSGVHTFPAVLQSSGLYSLSSVVTVPSSNFGTQTYTCNVDHKPSNTKVDKTVERKCC
VECPPCPAPPVAGPSVFLFPPKPKDTLMISRTPEVTCVVVDVSHEDPEVQFNWYVDGVEV
HNAKTKPREEQFNSTFRVVSVLTVVHQDWLNGKEYKCKVSNKGLPAPIEKTISKTKGQPR
EPQVYTLPPSREEMTKNQVSLTCLVKGFYPSDIAVEWESNGQPENNYKTTPPMLDSDGSF
FLYSKLTVDKSRWQQGNVFSCSVMHEALHNHYTQKSLSLSPGK
OMP-18R5 Light chain amino acid sequence with predicted signal
sequence underlined
(SEQ ID NO: 10)
MAWALLLLTLLTQGTGSWADIELTQPPSVSVAPGQTARISCSGDNIGSFYVHWYQQKPGQ
APVLVIYDKSNRPSGIPERFSGSNSGNTATLTISGTQAEDEADYYCQSYANTLSLVFGGG
TKLTVLGQPKAAPSVTLFPPSSEELQANKATLVCLISDFYPGAVTVAWKADSSPVKAGVE
TTTPSKQSNNKYAASSYLSLTPEQWKSHRSYSCQVTHEGSTVEKTVAPTECS
OMP-18R5 Heavy chain amino acid sequence without predicted
signal sequence
(SEQ ID NO: 11)
EVQLVESGGGLVQPGGSLRLSCAASGFTFSHYTLSWVRQAPGKGLEWVSVISGDGSYTYY
ADSVKGRFTISSDNSKNTLYLQMNSLRAEDTAVYYCARNFIKYVFANWGQGTLVTVSSAS
TKGPSVFPLAPCSRSTSESTAALGCLVKDYFPEPVTVSWNSGALTSGVHTFPAVLQSSGL
YSLSSVVTVPSSNFGTQTYTCNVDHKPSNTKVDKTVERKCCVECPPCPAPPVAGPSVFLF
PPKPKDTLMISRTPEVTCVVVDVSHEDPEVQFNWYVDGVEVHNAKTKPREEQFNSTFRVV
SVLTVVHQDWLNGKEYKCKVSNKGLPAPIEKTISKTKGQPREPQVYTLPPSREEMTKNQV
SLTCLVKGFYPSDIAVEWESNGQPENNYKTTPPMLDSDGSFFLYSKLTVDKSRWQQGNVF
SCSVMHEALHNHYTQKSLSLSPGK
OMP-18R5 Light chain amino acid sequence without predicted
signal sequence
(SEQ ID NO: 12)
DIELTQPPSVSVAPGQTARISCSGDNIGSFYVHWYQQKPGQAPVLVIYDKSNRPSGIPER
FSGSNSGNTATLTISGTQAEDEADYYCQSYANTLSLVFGGGTKLTVLGQPKAAPSVTLFP
PSSEELQANKATLVCLISDFYPGAVTVAWKADSSPVKAGVETTTPSKQSNNKYAASSYLS
LTPEQWKSHRSYSCQVTHEGSTVEKTVAPTECS
Human FZD1 Fri domain amino acid sequence without predicted
signal sequence
(SEQ ID NO: 13)
QQPPPPPQQQQSGQQYNGERGISVPDHGYCQPISIPLCTDIAYNQTIMPNLLGHTNQEDA
GLEVHQFYPLVKVQCSAELKFFLCSMYAPVCTVLEQALPPCRSLCERARQGCEALMNKFG
FQWPDTLKCEKFPVHGAGELCVGQNTSDKGT
Human FZD2 Fri domain amino acid sequence without predicted
signal sequence
(SEQ ID NO: 14)
QFHGEKGISIPDHGFCQPISIPLCTDIAYNQTIMPNLLGHTNQEDAGLEVHQFYPLVKVQ
CSPELRFFLCSMYAPVCTVLEQAIPPCRSICERARQGCEALMNKFGFQWPERLRCEHFPR
HGAEQICVGQNHSEDG
Human FZD3 Fri domain amino acid sequence without predicted
signal sequence
(SEQ ID NO: 15)
HSLFSCEPITLRMCQDLPYNTTFMPNLLNHYDQQTAALAMEPFHPMVNLDCSRDF
RPFLCALYAPICMEYGRVTLPCRRLCQRAYSECSKLMEMFGVPWPEDMECSRFPDCDEPY
PRLVDL
Human FZD4 Fri domain amino acid sequence without predicted
signal sequence
(SEQ ID NO: 16)
FGDEEERRCDPIRISMCQNLGYNVTKMPNLVGHELQTDAELQLTTFTPLIQYGCSSQLQF
FLCSVYVPMCTEKINIPIGPCGGMCLSVKRRCEPVLKEFGFAWPESLNCSKFPPQNDHNH
MCMEGPGDEEV
Human FZD5 Fri domain amino acid sequence without predicted
signal sequence
(SEQ ID NO: 17)
ASKAPVCQEITVPMCRGIGYNLTHMPNQFNHDTQDEAGLEVHQFWPLVEIQCSPDLRFFL
CSMYTPICLPDYHKPLPPCRSVCERAKAGCSPLMRQYGFAWPERMSCDRLPVLGRDAEVL
CMDYNRSEATT
Human FZD6 Fri domain amino acid sequence without predicted
signal sequence
(SEQ ID NO: 18)
HSLFTCEPITVPRCMKMAYNMTFFPNLMGHYDQSIAAVEMEHFLPLANLECSPNIETFLC
KAFVPTCIEQIHVVPPCRKLCEKVYSDCKKLIDTFGIRWPEELECDRLQYCDETVPVTFD
PHTEFLG
Human FZD7 Fri domain amino acid sequence without predicted
signal sequence
(SEQ ID NO: 19)
QPYHGEKGISVPDHGFCQPISIPLCTDIAYNQTILPNLLGHTNQEDAGLEVHQFYPLVKV
QCSPELRFFLCSMYAPVCTVLDQAIPPCRSLCERARQGCEALMNKFGFQWPERLRCENFP
VHGAGEICVGQNTSDGSG
Human FZD8 Fri domain amino acid sequence without predicted
signal sequence
(SEQ ID NO: 20)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTT
Human FZD9 Fri domain amino acid sequence without predicted
signal sequence
(SEQ ID NO: 21)
LEIGRFDPERGRGAAPCQAVEIPMCRGIGYNLTRMPNLLGHTSQGEAAAELAEFAPLVQY
GCHSHLRFFLCSLYAPMCTDQVSTPIPACRPMCEQARLRCAPIMEQFNFGWPDSLDCARL
PTRNDPHALCMEAPENA
Human FZD10 Fri domain amino acid sequence without predicted
signal sequence
(SEQ ID NO: 22)
ISSMDMERPGDGKCQPIEIPMCKDIGYNMTRMPNLMGHENQREAAIQLHEFAPLVEYGCH
GHLRFFLCSLYAPMCTEQVSTPIPACRVMCEQARLKCSPIMEQFNFKWPDSLDCRKLPNK
NDPNYLCMEAPNNG
Human FZD1 amino acids 116-227
(SEQ ID NO: 23)
CQPISIPLCTDIAYNQTIMPNLLGHTNQEDAGLEVHQFYPLVKVQCSAELKFFLCSMYAP
VCTVLEQALPPCRSLCERARQGCEALMNKFGFQWPDTLKCEKFPVHGAGELC
Human FZD2 amino acids 39-150
(SEQ ID NO: 24)
CQPISIPLCTDIAYNQTIMPNLLGHTNQEDAGLEVHQFYPLVKVQCSPELRFFLCSMYAP
VCTVLEQAIPPCRSICERARQGCEALMNKFGFQWPERLRCEHFPRHGAEQIC
Human FZD3 amino acids 28-133
(SEQ ID NO: 25)
CEPITLRMCQDLPYNTTFMPNLLNHYDQQTAALAMEPFHPMVNLDCSRDFRPFLCALYAP
ICMEYGRVTLPCRRLCQRAYSECSKLMEMFGVPWPEDMECSRFPDC
Human FZD4 amino acids 48-161
(SEQ ID NO: 26)
CDPIRISMCQNLGYNVTKMPNLVGHELQTDAELQLTTFTPLIQYGCSSQLQFFLCSVYVP
MCTEKINIPIGPCGGMCLSVKRRCEPVLKEFGFAWPESLNCSKFPPQNDHNHMC
Human FZD5 amino acids 33-147
(SEQ ID NO: 27)
CQEITVPMCRGIGYNLTHMPNQFNHDTQDEAGLEVHQFWPLVEIQCSPDLRFFLCSMYTP
ICLPDYHKPLPPCRSVCERAKAGCSPLMRQYGFAWPERMSCDRLPVLGRDAEVLC
Human FZD6 amino acids 24-129
(SEQ ID NO: 28)
CEPITVPRCMKMAYNMTFFPNLMGHYDQSIAAVEMEHFLPLANLECSPNIETFLCKAFVP
TCIEQIHVVPPCRKLCEKVYSDCKKLIDTFGIRWPEELECDRLQYC
Human FZD7 amino acids 49-160
(SEQ ID NO: 28)
CQPISIPLCTDIAYNQTILPNLLGHTNQEDAGLEVHQFYPLVKVQCSPELRFFLCSMYAP
VCTVLDQAIPPCRSLCERARQGCEALMNKFGFQWPERLRCENFPVHGAGEIC
Human FZD8 amino acids 35-148
(SEQ ID NO: 30)
CQEITVPLCKGIGYNYTYMPNQFNHDTQDEAGLEVHQFWPLVEIQCSPDLKFFLCSMYTP
ICLEDYKKPLPPCRSVCERAKAGCAPLMRQYGFAWPDRMRCDRLPEQGNPDTLC
Human FZD9 amino acids 39-152
(SEQ ID NO: 31)
CQAVEIPMCRGIGYNLTRMPNLLGHTSQGEAAAELAEFAPLVQYGCHSHLRFFLCSLYAP
MCTDQVSTPIPACRPMCEQARLRCAPIMEQFNFGWPDSLDCARLPTRNDPHALC
Human FZD10 amino acids 34-147
(SEQ ID NO: 32)
CQPIEIPMCKDIGYNMTRMPNLMGHENQREAAIQLHEFAPLVEYGCHGHLRFFLCSLYAP
MCTEQVSTPIPACRVMCEQARLKCSPIMEQFNFKWPDSLDCRKLPNKNDPNYLC
Human FZD8 Fri domain amino acid sequence without predicted
signal sequence (variant)
(SEQ ID NO: 33)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDL
Human IgG₁ Fc region
(SEQ ID NO: 34)
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
Human IgG₁ Fc region (variant)
(SEQ ID NO: 35)
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
Human IgG₁ Fc region
(SEQ ID NO: 36)
KSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
Human IgG₁ Fc region
(SEQ ID NO: 37)
EPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
Human IgG₂ Fc region
(SEQ ID NO: 38)
CVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDSDGS
FFLYSKLTVDKSRWQQGNVFSCSVMHEALHNHYTQKSLSLSPGK
FZD8-Fc variant 54F03 amino acid sequence
(without predicted signal sequence)
(SEQ ID NO: 39)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTTGRADKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
K
FZD8-Fc variant 54F16, 54F17, 54F18, 54F23, 54F25, 54F27, 54F29,
54F31, and 54F34 amino acid sequence
(without predicted signal sequence)
(SEQ ID NO: 40)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTTKSSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
K
FZD8-Fc variant 54F19, 54F20, 54F24, 54F26, 54F28, 54F30, 54F32,
54F34 and 54F35 amino acid sequence
(without predicted signal sequence)
(SEQ ID NO: 41)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTTEPKSSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLS
PGK
FZD8-Fc variant 54F03 amino acid sequence with signal sequence
(SEQ ID NO: 42)
MEWGYLLEVTSLLAALALLQRSSGAAAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTGRADKTHTCPPCPAPELLGGPS
VFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSRWQQ
GNVFSCSVMHEALHNHYTQKSLSLSPGK
FZD8-Fc variant 54F16 amino acid sequence with signal sequence
(SEQ ID NO: 43)
MEWGYLLEVTSLLAALALLQRSSGAAAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTKSSDKTHTCPPCPAPELLGGPS
VFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSRWQQ
GNVFSCSVMHEALHNHYTQKSLSLSPGK
FZD8-Fc variant 54F26 with signal sequence
(SEQ ID NO: 44)
MEWGYLLEVTSLLAALFLLQRSPIVHAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTEPKSSDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
FZD8-Fc variant 54F28 with signal sequence
(SEQ ID NO: 45)
MEWGYLLEVTSLLAALLLLQRSPFVHAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTEPKSSDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human Wnt1 C-terminal cysteine rich domain (aa 288-370)
(SEQ ID NO: 46)
DLVYFEKSPNFCTYSGRLGTAGTAGRACNSSSPALDGCELLCCGRGHRTRTQRVTERCNC
TFHWCCHVSCRNCTHTRVLHECL
Human Wnt2 C-terminal cysteine rich domain (aa 267-360)
(SEQ ID NO: 47)
DLVYFENSPDYCIRDREAGSLGTAGRVCNLTSRGMDSCEVMCCGRGYDTSHVTRMTKCGC
KFHWCCAVRCQDCLEALDVHTCKAPKNADWTTAT
Human Wnt2b C-terminal cysteine rich domain (aa 298-391)
(SEQ ID NO: 48)
DLVYFDNSPDYCVLDKAAGSLGTAGRVCSKTSKGTDGCEIMCCGRGYDTTRVTRVTQCEC
KFHWCCAVRCKECRNTVDVHTCKAPKKAEWLDQT
Human Wnt3 C-terminal cysteine rich domain (aa 273-355)
(SEQ ID NO: 49)
DLVYYENSPNFCEPNPETGSFGTRDRTCNVTSHGIDGCDLLCCGRGHNTRTEKRKEKCHC
IFHWCCYVSCQECIRIYDVHTCK
Human Wnt3a C-terminal cysteine rich domain (aa 270-352)
(SEQ ID NO: 50)
DLVYYEASPNFCEPNPETGSFGTRDRTCNVSSHGIDGCDLLCCGRGHNARAERRREKCRC
VFHWCCYVSCQECTRVYDVHTCK
Human Wnt7a C-terminal cysteine rich domain (aa 267-359)
(SEQ ID NO: 51)
DLVYIEKSPNYCEEDPVTGSVGTQGRACNKTAPQASGCDLMCCGRGYNTHQYARVWQCNC
KFHWCCYVKCNTCSERTEMYTCK
Human Wnt7b C-terminal cysteine rich domain (aa 267-349)
(SEQ ID NO: 52)
DLVYIEKSPNYCEEDAATGSVGTQGRLCNRTSPGADGCDTMCCGRGYNTHQYTKVWQCNC
KFHWCCFVKCNTCSERTEVFTCK
Human Wnt8a C-terminal cysteine rich domain (aa 248-355)
(SEQ ID NO: 53)
ELIFLEESPDYCTCNSSLGIYGTEGRECLQNSHNTSRWERRSCGRLCTECGLQVEERKTE
VISSCNCKFQWCCTVKCDQCRHVVSKYYCARSPGSAQSLGRVWFGVYI
Human Wnt8b C-terminal cysteine rich domain (aa 245-351)
(SEQ ID NO: 54)
ELVHLEDSPDYCLENKTLGLLGTEGRECLRRGRALGRWELRSCRRLCGDCGLAVEERRAE
TVSSCNCKFHWCCAVRCEQCRRRVTKYFCSRAERPRGGAAHKPGRKP
Human Wnt10a C-terminal cysteine rich domain (aa 335-417)
(SEQ ID NO: 55)
DLVYFEKSPDFCEREPRLDSAGTVGRLCNKSSAGSDGCGSMCCGRGHNILRQTRSERCHC
RFHWCCFVVCEECRITEWVSVCK
Human Wnt10b C-terminal cysteine rich domain (aa 307-389)
(SEQ ID NO: 56)
ELVYFEKSPDFCERDPTMGSPGTRGRACNKTSRLLDGCGSLCCGRGHNVLRQTRVERCHC
RFHWCCYVLCDECKVTEWVNVCK
Linker
(SEQ ID NO: 57)
ESGGGGVT
Linker
(SEQ ID NO: 58)
LESGGGGVT
Linker
(SEQ ID NO: 59)
GRAQVT
Linker
(SEQ ID NO: 60)
WRAQVT
Linker
(SEQ ID NO: 61)
ARGRAQVT
CCND2 Forward Primer
(SEQ ID NO: 62)
GCTGTCTCTGATCCGCAAGC
CCND2 Reverse Primer
(SEQ ID NO: 63)
GACGGTGGGTACATGGCAAAC
CCND2 Probe
(SEQ ID NO: 64)
CCTTCATTGCTCTGTGTGCCACCGAC
CTBP2 Forward Primer
(SEQ ID NO: 65)
ATCCGTGGGGAGACGCTG
CTBP2 Reverse Primer
(SEQ ID NO: 66)
CTCGAACTGCAACCGCCTG
CTBP2 Probe
(SEQ ID NO: 67)
CCCGTGCGACCAAAGCCAATGAGG
DKK1 Forward Primer
(SEQ ID NO: 68)
GACCATTGACAACTACCAGCCGTA
DKK1 Reverse Primer
(SEQ ID NO: 69)
TGGGACTAGCGCAGTACTCATC
DKK1 Probe
(SEQ ID NO: 70)
TGCCGCACTCCTCGTCCTCTG
FBXW2 Forward Primer
(SEQ ID NO: 71)
GCCAGTTATGATATTCTCAGGGTCA
FBXW2 Reverse Primer
(SEQ ID NO: 72)
AGCAGGGCAAAGATATCTCCAAA
FBXW2 Probe
(SEQ ID NO: 73)
AGACTCCTGAGATAGCAAACTTGGCCT
RHOU1 Forward Primer
(SEQ ID NO: 74)
CCCACCGAGTACATCCCTACTG
RHOU1 Reverse Primer
(SEQ ID NO: 75)
CAGTGTCACAGAGTTGGAGTCTCA
RHOU1 Probe
(SEQ ID NO: 76)
CGCCCATCCACAGACACCACCG
WIF1 Forward Primer
(SEQ ID NO: 77)
GTTCCAAAGGTTACCAGGGAGAC
WIF1 Reverse Primer
(SEQ ID NO: 78)
GTTGGGTTCATGGCAGGTTCC
WIF1 Probe
(SEQ ID NO: 79)
CCAGGCTCGCAGACAGGCTTTGAAC

What is claimed is:

1. A method of identifying a human tumor that is likely to be responsive or non-responsive to treatment with a Wnt pathway inhibitor, the method comprising:
(a) obtaining a sample of the human tumor;
(b) measuring the expression level of each biomarker of a biomarker signature in the sample, wherein the signature comprises one or more of the biomarkers FBXW2, CCND2, RHOU, CTBP2, WIF1, and DKK1; and
(c) identifying the tumor as likely to be responsive or non-responsive to treatment based upon the expression level of the biomarkers.

2. A method of identifying a human tumor that is likely to be responsive or non-responsive to treatment with a Wnt pathway inhibitor, the method comprising:
(a) obtaining a sample of the human tumor;
(b) measuring the expression level of each biomarker of a biomarker signature in the sample, wherein the signature comprises one or more of the biomarkers FBXW2, CCND2, RHOU, CTBP2, WIF1, and DKK1; and
(c) calculating a decision value based upon the standardized expression of the biomarkers in the signature;
wherein a positive decision value indicates the tumor is predicted to be responsive to the Wnt pathway inhibitor and a negative decision value indicates the tumor is predicted to be non-responsive to the Wnt pathway inhibitor.

3. A method of classifying a human tumor as likely to be responsive or non-responsive to treatment with a Wnt pathway inhibitor, the method comprising:
(a) obtaining a sample of the human tumor;
(b) measuring the expression level of each biomarker of a biomarker signature in the sample, wherein the signature comprises one or more of the biomarkers FBXW2, CCND2, RHOU, CTBP2, WIF1, and DKK1; and
(c) classifying the tumor as likely to be responsive or non-responsive to treatment based upon the expression of the biomarkers.

4. A method of classifying a human tumor as likely to be responsive or non-responsive to treatment with a Wnt pathway inhibitor, the method comprising:
(a) obtaining a sample of the human tumor;
(b) measuring the expression level of each biomarker of a biomarker signature in the sample, wherein the signature comprises one or more of the biomarkers FBXW2, CCND2, RHOU, CTBP2, WIF1, and DKK1; and
(c) calculating a decision value based upon the standardized expression of the biomarkers in the signature;
wherein a positive decision value indicates the tumor is predicted to be responsive the Wnt pathway inhibitor and a negative decision value indicates the tumor is predicted to be non-responsive the Wnt pathway inhibitor.

5. A method of determining the responsiveness of a human tumor to treatment with a Wnt pathway inhibitor, the method comprising:
(a) obtaining a sample of the human tumor;
(b) measuring the expression level of each biomarker of a biomarker signature in the sample, wherein the signature comprises one or more of the biomarkers FBXW2, CCND2, RHOU, CTBP2, WIF1, and DKK1; and
(c) determining the responsiveness of the tumor to treatment based upon the expression of the biomarkers.

6. A method of determining the responsiveness of a human tumor to treatment with a Wnt pathway inhibitor, the method comprising:
(a) obtaining a sample of the human tumor;
(b) measuring the expression level of each biomarker of a biomarker signature in the sample, wherein the signature comprises one or more of the biomarkers FBXW2, CCND2, RHOU, CTBP2, WIF1, and DKK1; and
(c) calculating a decision value based upon the standardized expression of the biomarkers in the signature;
wherein a positive decision value indicates the tumor is predicted to be responsive to the Wnt pathway inhibitor.

7. A method of identifying a patient with cancer who is likely to respond to treatment with a Wnt pathway inhibitor, the method comprising:
(a) obtaining a sample of the patient's tumor;
(b) measuring the expression level of each biomarker of a biomarker signature in the sample, wherein the signature comprises one or more of the biomarkers FBXW2, CCND2, RHOU, CTBP2, WIF1, and DKK1; and
(c) identifying the patient who is likely to respond to treatment based upon the expression level of the biomarkers.

8. A method of identifying a patient with cancer who is likely to respond to treatment with a Wnt pathway inhibitor, the method comprising:
(a) obtaining a sample of the patient's tumor;
(b) measuring the expression level of each biomarker of a biomarker signature in the sample, wherein the signature comprises one or more of the biomarkers FBXW2, CCND2, RHOU, CTBP2, WIF1, and DKK1; and
(c) calculating a decision value based upon the standardized expression of the biomarkers in the signature;
wherein a positive decision value indicates that the patient is predicted to respond to treatment with the Wnt pathway inhibitor.

9. A method of selecting a patient with cancer for treatment with a Wnt pathway inhibitor, the method comprising:
(a) obtaining a sample of the patient's tumor;
(b) measuring the expression level of each biomarker of a biomarker signature in the sample, wherein the biomarker signature comprises one or more of the biomarkers FBXW2, CCND2, RHOU, CTBP2, WIF1, and DKK1; and
(c) selecting the patient for treatment based upon the expression level of the biomarkers.

10. A method of selecting a patient with cancer for treatment with a Wnt pathway inhibitor, the method comprising:
(a) obtaining a sample of the patient's tumor;
(b) measuring the expression level of each biomarker of a biomarker signature in the sample, wherein the biomarker signature comprises one or more of the biomarkers FBXW2, CCND2, RHOU, CTBP2, WIF1, and DKK1;
(c) calculating a decision value based upon the standardized expression of the biomarkers in the biomarker signature; and
(d) selecting the patient for treatment when their tumor sample has a positive decision value.

11. A method of treating cancer in a patient, comprising:
(a) identifying if the patient is likely to respond to treatment with a Wnt pathway inhibitor, wherein the identification comprises:
(i) obtaining a sample of the patient's tumor;
(ii) measuring the expression level of each biomarker of a biomarker signature in the sample, wherein the signature comprises one or more of the biomarkers FBXW2, CCND2, RHOU, CTBP2, WIF1, and DKK1; and
(iii) identifying the patient who is likely to respond to treatment based upon the expression level of the biomarkers; and
(b) administering an effective amount of a Wnt pathway inhibitor to the patient who is likely to response to treatment.

12. A method of treating cancer in a patient, comprising:
(a) identifying if the patient is likely to respond to treatment with a Wnt pathway inhibitor, wherein the identification comprises:
(i) obtaining a sample of the patient's tumor;
(ii) measuring the expression level of each biomarker of a biomarker signature in the sample, wherein the signature comprises one or more of the biomarkers FBXW2, CCND2, RHOU, CTBP2, WIF1, and DKK1; and
(iii) calculating a decision value based upon the standardized expression of the biomarkers in the signature;
wherein a positive decision value indicates that a patient is predicted to respond to treatment with the Wnt pathway inhibitor; and
(b) administering an effective amount of a Wnt pathway inhibitor to the patient who is predicted to response to treatment.

13. A method for increasing the likelihood of effective treatment with a Wnt pathway inhibitor, comprising:
(a) identifying if a patient has a tumor that is likely to respond to treatment with a Wnt pathway inhibitor, wherein the identification comprises:
(i) obtaining a sample of the patient's cancer;
(ii) measuring the expression level of each biomarker of a biomarker signature in the sample, wherein the signature comprises one or more of the biomarkers FBXW2, CCND2, RHOU, CTBP2, WIF1, and DKK1; and
(iii) identifying the patient who is likely to respond to treatment based upon the expression level of the biomarkers; and
(b) administering an effective amount of the Wnt pathway inhibitor to the patient who is likely to respond to treatment.

14. A method for increasing the likelihood of effective treatment with a Wnt pathway inhibitor, comprising:
(a) identifying if a patient has a tumor that is likely to respond to treatment with a Wnt pathway inhibitor, wherein the identification comprises:
(i) obtaining a sample of the patient's cancer;
(ii) measuring the expression level of each biomarker of a biomarker signature in the sample, wherein the signature comprises one or more of the biomarkers FBXW2, CCND2, RHOU, CTBP2, WIF1, and DKK1; and
(iii) calculating a decision value based upon the standardized expression of the biomarkers in the signature;
wherein a positive decision value indicates that a patient is predicted to respond to treatment with the Wnt pathway inhibitor; and
(b) administering an effective amount of the WNT pathway inhibitor to the patient whose tumor has a positive decision value.

15. The method according to any one of claims 1-14, wherein the biomarker signature comprises one or more of the biomarkers FBXW2, CCND2, RHOU, CTBP2, WIF1, DKK1, EP300, CTBP1, WNT6, WNT3, FZD2, APC, TLE2, DVL2, PITX2, WISP1, GSK3B, WNT9A, FZD7, and LEF1.

16. The method according to any one of claims 1-15, wherein the biomarker signature comprises one or more of the biomarkers FBXW2, CCND2, RHOU, CTBP2, WIF1, DKK1, EP300, and CTBP1.

17. The method according to any one of claims 1-16, wherein the biomarker signature comprises two or more of the biomarkers FBXW2, CCND2, RHOU, CTBP2, WIF1, and DKK1.

18. The method according to any one of claims 1-16, wherein the biomarker signature comprises three or more of the biomarkers FBXW2, CCND2, RHOU, CTBP2, WIF1, and DKK1.

19. The method according to any one of claims 1-16, wherein the biomarker signature comprises four or more of the biomarkers FBXW2, CCND2, RHOU, CTBP2, WIF1, and DKK1.

20. The method according to any one of claims 1-16, wherein the biomarker signature comprises five of the biomarkers FBXW2, CCND2, RHOU, CTBP2, WIF1, and DKK1.

21. The method according to any one of claims 1-16, wherein the biomarker signature comprises the biomarkers FBXW2, CCND2, RHOU, CTBP2, WIF1, and DKK1.

22. The method according to any one of claims 1-16, wherein the biomarker signature consists of the biomarkers FBXW2, CCND2, RHOU, CTBP2, WIF1, and DKK1.

23. The method according to any one of claims 1-22, wherein the expression of each biomarker is measured by a PCR-based assay.

24. The method according to any one of claims 1-23, wherein the expression of each biomarker is measured by a qPCR assay.

25. The method according to any one of claims 1-22, wherein the expression of each biomarker is measured by a microarray.

26. The method according to any one of claims 1-25, wherein the standardized expression of each biomarker is determined by measuring an expression level for each biomarker and multiplying it by a corresponding weight, wherein the weight for each biomarker is determined by the expression signature.

27. The method according to any one of claims 1-26, wherein the decision value is calculated according to the equation: 0.4560427*FBXW2+0.3378467*CCND2−0.4809354*RHOU+0.409029*CTBP2+0.3291529*WIF1+0.2926374*DKK1+0.04662682.

28. The method according to any one of claims 1-25, wherein the expression levels of FBXW2, CCND2, RHOU, CTBP2, WIF1, and DKK1 are measured using polynucleotides selected from the group consisting of SEQ ID NOs:62-79.

29. The method of claim 28, wherein the expression levels of FBXW2, CCND2, RHOU, CTBP2, WIF1, and DKK1 are measured using:
(a) a forward primer of SEQ ID NO:62, a reverse primer of SEQ ID NO:63, and a probe comprising SEQ ID NO:64;
(b) a forward primer of SEQ ID NO:65, a reverse primer of SEQ ID NO:66, and a probe comprising SEQ ID NO:67;
(c) a forward primer of SEQ ID NO:68, a reverse primer of SEQ ID NO:69, and a probe comprising SEQ ID NO:70;
(d) a forward primer of SEQ ID NO:71, a reverse primer of SEQ ID NO:72, and a probe comprising SEQ ID NO:73;
(e) a forward primer of SEQ ID NO:74, a reverse primer of SEQ ID NO:75, and a probe comprising SEQ ID NO:76; and
(f) a forward primer of SEQ ID NO:77, a reverse primer of SEQ ID NO:78, and a probe comprising SEQ ID NO:79.

30. The method according to any one of claims 1-29, wherein the Wnt pathway inhibitor is an antibody.

31. The method according to any one of claims 1-30, wherein the Wnt pathway inhibitor is an antibody that specifically binds at least one Frizzled (FZD) protein or portion thereof.

32. The method of claim 30 or claim 31, wherein the antibody specifically binds at least one FZD protein selected from the group consisting of: FZD1, FZD2, FZD3, FZD4, FZD5, FZD6, FZD7, FZD8, FZD9, and FZD10.

33. The method of claim 30 or claim 31, wherein the antibody specifically binds at least one FZD protein selected from the group consisting of: FZD1, FZD2, FZD5, FZD7, and FZD8.

34. The method according to any one of claims 1-33, wherein the Wnt pathway inhibitor is an antibody comprising:
(a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and
(b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6).

35. The method according to any one of claims 1-34, wherein the Wnt pathway inhibitor is an antibody comprising a heavy chain variable region comprising SEQ ID NO:7 and a light chain variable region comprising SEQ ID NO:8.

36. The method according to any one of claims 1-34, wherein the Wnt pathway inhibitor is an antibody comprising a heavy chain variable region and a light chain variable region encoded by the plasmid deposited with ATCC as PTA-9541.

37. The method according to any one of claims 30-36, wherein the antibody is a monoclonal antibody, a recombinant antibody, a chimeric antibody, a bispecific antibody, a humanized antibody, a human antibody, or a antibody fragment comprising an antigen-binding site.

38. The method according to any one of claims 1-35, wherein the Wnt pathway inhibitor is antibody OMP-18R5.

39. The method according to any one of claims 1-29, wherein the Wnt pathway inhibitor is a soluble receptor.

40. The method of claim 39, wherein the soluble receptor comprises a Fri domain of a human FZD protein.

41. The method of claim 38, wherein the Fri domain of the human FZD protein is selected from the group consisting of: the Fri domain of FZD 1, the Fri domain of FZD2, the Fri domain of FZD3, the Fri domain of FZD4, the Fri domain of FZD5, the Fri domain of FZD6, the Fri domain of FZD7, the Fri domain of FZD8, the Fri domain of FZD9, or the Fri domain of FZD10.

42. The method of claim 40, wherein the Fri domain of the human FZD protein comprises the Fri domain of FZD8.

43. The method according to any one of claims 39-42, wherein the soluble receptor further comprises a non-FZD polypeptide.

44. The method of claim 43, wherein the non-FZD polypeptide comprises a human Fc region.

45. The method according to any one of claims 39-44, wherein the Wnt pathway inhibitor is FZD8-Fc soluble receptor OMP-54F28.

46. The method according to any one of claims 1-45, wherein the tumor is selected from the group consisting of: a breast tumor, a lung tumor, a colon tumor, a colorectal tumor, a melanoma, a pancreatic tumor, a gastrointestinal tumor, a renal tumor, an ovarian tumor, a neuroendocrine tumor, a liver tumor, an endometrial tumor, a kidney tumor, a prostate tumor, a thyroid tumor, a neuroblastoma, a glioma, a glioblastoma multiforme, a cervical tumor, a stomach tumor, a bladder tumor, a hepatoma, and a head and neck tumor.

47. The method of according to any one of claims 1-45, wherein the tumor is a breast tumor.

48. The method of claim 47, wherein the breast tumor is a HER2-negative breast tumor.

49. The method of claim 47, wherein the breast tumor is a triple negative breast cancer (TNBC) tumor.

50. The method according to any one of claims 1-49, wherein the treatment with a Wnt pathway inhibitor is in combination with one or more additional therapeutic agents.

51. The method of claim 50, wherein the additional therapeutic agent is a chemotherapeutic agent.

52. The method of claim 50, wherein the additional therapeutic agent is paclitaxel.

53. The method of claim 50, wherein the additional therapeutic agent is nab-bound paclitaxel (ABRAXANE).

54. The method according to any one of claims 1-53, wherein the sample is a tissue sample or a tumor biopsy.

55. The method according to any one of claims 1-53, wherein the sample is a formalin-fixed paraffin embedded (FFPE) sample.

56. A method of identifying a human breast tumor that is likely to be responsive to or non-responsive to treatment with an antibody that specifically binds at least one human frizzled (FZD) selected from the group consisting of FZD1, FZD2, FZD5, FZD7, and FZD8, the method comprising:
(a) obtaining a sample of the human breast tumor;
(b) measuring the biomarker expression level of each biomarker of a biomarker signature in the sample, wherein the biomarker signature comprises one or more of the biomarkers FBXW2, CCND2, RHOU, CTBP2, WIF1, and DKK1; and
(c) calculating a decision value based upon the standardized expression of the biomarkers in the biomarker signature;
wherein a positive decision value indicates the breast tumor is predicted to be responsive to treatment with the antibody and a negative decision value indicates the tumor is predicted to be non-responsive to treatment with the antibody.

57. A method of identifying a patient with breast cancer that is likely to be responsive to treatment with an antibody that specifically binds at least one human frizzled (FZD) selected from the group consisting of FZD1, FZD2, FZD5, FZD7, and FZD8, the method comprising:
(a) obtaining a sample of the breast tumor;
(b) measuring the biomarker expression level of each biomarker of a biomarker signature in the sample, wherein the biomarker signature comprises one or more of the biomarkers FBXW2, CCND2, RHOU, CTBP2, WIF1, and DKK1; and
(c) calculating a decision value based upon the standardized expression of the biomarkers in the biomarker signature;
wherein a positive decision value indicates the breast cancer is predicted to be responsive to treatment with the antibody.

58. A method of selecting a patient with breast cancer that is likely to be responsive to treatment with an antibody that specifically binds at least one human frizzled (FZD) selected from the group consisting of FZD 1, FZD2, FZD5, FZD7, and FZD8, the method comprising:
(a) obtaining a sample of the breast tumor;
(b) measuring the biomarker expression level of each biomarker of a biomarker signature in the sample, wherein the biomarker signature comprises one or more of the biomarkers FBXW2, CCND2, RHOU, CTBP2, WIF1, and DKK1;
(c) calculating a decision value based upon the standardized expression of the biomarkers in the biomarker signature;
wherein a positive decision value indicates the breast cancer is predicted to be responsive to treatment with the antibody; and
(d) selecting the patient for treatment when their tumor sample has a positive decision value.

59. The method of claim 56 or claim 57, further comprising:
(d) selecting a patient for treatment when the breast cancer is predicted to be responsive to treatment with the antibody.

60. The method according to any one of claims 56-59, further comprising administering an effective therapeutic amount of the antibody to the patient.

61. The method of claim 60, wherein the antibody is OMP-18R5.

62. The method of claim 56-61, wherein the treatment comprises the antibody in combination with paclitaxel.

63. A method of treating cancer in a patient, comprising: administering an effective amount of a Wnt pathway inhibitor to the patient, wherein the patient is predicted to respond to treatment with the Wnt pathway inhibitor based upon expression levels of a biomarker signature in a patient tumor sample, wherein the signature comprises one or more of the biomarkers FBXW2, CCND2, RHOU, CTBP2, WIF1, and DKK1.

64. A kit for detecting FBXW2, CCND2, RHOU, CTBP2, WIF1, and DKK1 in a sample, wherein the kit comprises polynucleotides selected from the group consisting of SEQ ID NOs:62-79.

65. The kit of claim 64, which comprises:
(a) a forward primer of SEQ ID NO:62, a reverse primer of SEQ ID NO:63, and a probe comprising SEQ ID NO:64;
(b) a forward primer of SEQ ID NO:65, a reverse primer of SEQ ID NO:66, and a probe comprising SEQ ID NO:67;
(c) a forward primer of SEQ ID NO:68, a reverse primer of SEQ ID NO:69, and a probe comprising SEQ ID NO:70;
(d) a forward primer of SEQ ID NO:71, a reverse primer of SEQ ID NO:72, and a probe comprising SEQ ID NO:73;
(e) a forward primer of SEQ ID NO:74, a reverse primer of SEQ ID NO:75, and a probe comprising SEQ ID NO:76; and
(f) a forward primer of SEQ ID NO:77, a reverse primer of SEQ ID NO:78, and a probe comprising SEQ ID NO:79.


SEQUENCE LISTING (79 sequences)

SEQ ID NO: 1 - protein, 10 aa, Artificial Sequence
  18R5 Heavy chain CDR1
GFTFSHYTLS

SEQ ID NO: 2 - protein, 17 aa, Artificial Sequence
  Heavy chain CDR2
VISGDGSYTYYADSVKG

SEQ ID NO: 3 - protein, 9 aa, Artificial Sequence
  Heavy chain CDR3
NFIKYVFAN

SEQ ID NO: 4 - protein, 11 aa, Artificial Sequence
  Light chain CDR1
SGDNIGSFYVH

SEQ ID NO: 5 - protein, 8 aa, Artificial Sequence
  Light chain CDR2
DKSNRPSG

SEQ ID NO: 6 - protein, 9 aa, Artificial Sequence
  Light chain CDR3
QSYANTLSL

SEQ ID NO: 7 - protein, 118 aa, Artificial Sequence
  Heavy chain variable region amino acid sequence
EVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSS

SEQ ID NO: 8 - protein, 108 aa, Artificial Sequence
  Light chain variable region amino acid sequence
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLG

SEQ ID NO: 9 - protein, 463 aa, Artificial Sequence
  Heavy chain amino acid sequence
MKHLWFFLLLVAAPRWVLSEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 232 aa, Artificial Sequence
  Light chain amino acid sequence
MAWALLLLTLLTQGTGSWADIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 444 aa, Artificial Sequence
  Heavy chain amino acid sequence without predicted signal sequence
EVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 213 aa, Artificial Sequence
  Light chain amino acid sequence without predicted signal sequence
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 151 aa, Artificial Sequence
  Fri domain amino acid sequence without predicted signal sequence
QQPPPPPQQQQSGQQYNGERGISVPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGT

SEQ ID NO: 14 - protein, 136 aa, Artificial Sequence
  Fri domain amino acid sequence without predicted signal sequence
QFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDG

SEQ ID NO: 15 - protein, 121 aa, Artificial Sequence
  Fri domain amino acid sequence without predicted signal sequence
HSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDCDEPYPRLVDL

SEQ ID NO: 16 - protein, 131 aa, Artificial Sequence
  Fri domain amino acid sequence without predicted signal sequence
FGDEEERRCDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMCMEGPGDEEV

SEQ ID NO: 17 - protein, 131 aa, Artificial Sequence
  Human FZD5 Fri domain amino acid sequence without predicted signal sequence
ASKAPVCQEITVPMCRGIGYNLTHMPNQFNHDTQDEAGLEVHQFWPLVEIQCSPDLRFFLCSMYTPICLPDYHKPLPPCRSVCERAKAGCSPLMRQYGFAWPERMSCDRLPVLGRDAEVLCMDYNRSEATT

SEQ ID NO: 18 - protein, 127 aa, Artificial Sequence
  Human FZD6 Fri domain amino acid sequence without predicted signal sequence
HSLFTCEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYCDETVPVTFDPHTEFLG

SEQ ID NO: 19 - protein, 138 aa, Artificial Sequence
  Human FZD7 Fri domain amino acid sequence without predicted signal sequence
QPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSG

SEQ ID NO: 20 - protein, 131 aa, Artificial Sequence
  Human FZD8 Fri domain amino acid sequence without predicted signal sequence
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTT

SEQ ID NO: 21 - protein, 137 aa, Artificial Sequence
  Human FZD9 Fri domain amino acid sequence without predicted signal sequence
LEIGRFDPERGRGAAPCQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALCMEAPENA

SEQ ID NO: 22 - protein, 134 aa, Artificial Sequence
  Human FZD10 Fri domain amino acid sequence without predicted signal sequence
ISSMDMERPGDGKCQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLCMEAPNNG

SEQ ID NO: 23 - protein, 112 aa, Artificial Sequence
  Human FZD1 amino acids 116-227
CQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELC

SEQ ID NO: 24 - protein, 112 aa, Artificial Sequence
  Human FZD2 amino acids 39-150
CQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQIC

SEQ ID NO: 25 - protein, 106 aa, Artificial Sequence
  Human FZD3 amino acids 28-133
CEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDC

SEQ ID NO: 26 - protein, 114 aa, Artificial Sequence
  Human FZD4 amino acids 48-161
CDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMC

SEQ ID NO: 27 - protein, 115 aa, Artificial Sequence
  Human FZD5 amino acids 33-147
CQEITVPMCRGIGYNLTHMPNQFNHDTQDEAGLEVHQFWPLVEIQCSPDLRFFLCSMYTPICLPDYHKPLPPCRSVCERAKAGCSPLMRQYGFAWPERMSCDRLPVLGRDAEVLC

SEQ ID NO: 28 - protein, 106 aa, Artificial Sequence
  Human FZD6 amino acids 24-129
CEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYC

SEQ ID NO: 29 - protein, 112 aa, Artificial Sequence
  Human FZD7 amino acids 49-160
CQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEIC

SEQ ID NO: 30 - protein, 114 aa, Artificial Sequence
  Human FZD8 amino acids 35-148
CQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLC

SEQ ID NO: 31 - protein, 114 aa, Artificial Sequence
  Human FZD9 amino acids 39-152
CQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALC

SEQ ID NO: 32 - protein, 114 aa, Artificial Sequence
  Human FZD10 amino acids 34-147
CQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLC

SEQ ID NO: 33 - protein, 129 aa, Artificial Sequence
  Human FZD8 Fri domain amino acid sequence without predicted signal sequence (variant)
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDL

SEQ ID NO: 34 - protein, 227 aa, Artificial Sequence
  Human IgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 227 aa, Artificial Sequence
  Human IgG1 Fc region (variant)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 230 aa, Artificial Sequence
  Human IgG1 Fc region
KSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 232 aa, Artificial Sequence
  Human IgG1 Fc region
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 224 aa, Artificial Sequence
  Human IgG2 Fc region
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 39 - protein, 361 aa, Artificial Sequence
  FZD8-Fc variant 54F03 amino acid sequence (without predicted signal sequence)
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTGRADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 361 aa, Artificial Sequence
  FZD8-Fc variant 54F16, 54F17, 54F18, 54F23, 54F25, 54F27, 54F29, 54F31, and 54F34 amino acid sequence (without predicted signal sequence)
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 363 aa, Artificial Sequence
  FZD8-Fc variant 54F19, 54F20, 54F24, 54F26, 54F28, 54F30, 54F32, 54F34 and 54F35 amino acid sequence (without predicted signal sequence)
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 388 aa, Artificial Sequence
  FZD8-Fc variant 54F03 amino acid sequence with signal sequence
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTGRADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 388 aa, Artificial Sequence
  FZD8-Fc variant 54F16 amino acid sequence with signal sequence
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 390 aa, Artificial Sequence
  FZD8-Fc variant 54F26 with signal sequence
MEWGYLLEVTSLLAALFLLQRSPIVHAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 390 aa, Artificial Sequence
  FZD8-Fc variant 54F28 with signal sequence
MEWGYLLEVTSLLAALLLLQRSPFVHAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 83 aa, Artificial Sequence
  Human Wnt1 C-terminal cysteine rich domain (aa 288-370)
DLVYFEKSPNFCTYSGRLGTAGTAGRACNSSSPALDGCELLCCGRGHRTRTQRVTERCNCTFHWCCHVSCRNCTHTRVLHECL

SEQ ID NO: 47 - protein, 94 aa, Artificial Sequence
  Human Wnt2 C-terminal cysteine rich domain (aa 267-360)
DLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 48 - protein, 94 aa, Artificial Sequence
  Human Wnt2b C-terminal cysteine rich domain (aa 298-391)
DLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 49 - protein, 83 aa, Artificial Sequence
  Human Wnt3 C-terminal cysteine rich domain (aa 273-355)
DLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 50 - protein, 83 aa, Artificial Sequence
  Human Wnt3a C-terminal cysteine rich domain (aa 270-352)
DLVYYEASPNFCEPNPETGSFGTRDRTCNVSSHGIDGCDLLCCGRGHNARAERRREKCRCVFHWCCYVSCQECTRVYDVHTCK

SEQ ID NO: 51 - protein, 83 aa, Artificial Sequence
  Human Wnt7a C-terminal cysteine rich domain (aa 267-359)
DLVYIEKSPNYCEEDPVTGSVGTQGRACNKTAPQASGCDLMCCGRGYNTHQYARVWQCNCKFHWCCYVKCNTCSERTEMYTCK

SEQ ID NO: 52 - protein, 83 aa, Artificial Sequence
  Human Wnt7b C-terminal cysteine rich domain (aa 267-349)
DLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 53 - protein, 108 aa, Artificial Sequence
  Human Wnt8a C-terminal cysteine rich domain (aa 248-355)
ELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGRVWFGVYI

SEQ ID NO: 54 - protein, 107 aa, Artificial Sequence
  Human Wnt8b C-terminal cysteine rich domain (aa 245-351)
ELVHLEDSPDYCLENKTLGLLGTEGRECLRRGRALGRWELRSCRRLCGDCGLAVEERRAETVSSCNCKFHWCCAVRCEQCRRRVTKYFCSRAERPRGGAAHKPGRKP

SEQ ID NO: 55 - protein, 83 aa, Artificial Sequence
  Human Wnt10a C-terminal cysteine rich domain (aa 335-417)
DLVYFEKSPDFCEREPRLDSAGTVGRLCNKSSAGSDGCGSMCCGRGHNILRQTRSERCHCRFHWCCFVVCEECRITEWVSVCK

SEQ ID NO: 56 - protein, 83 aa, Artificial Sequence
  Human Wnt10b C-terminal cysteine rich domain (aa 307-389)
ELVYFEKSPDFCERDPTMGSPGTRGRACNKTSRLLDGCGSLCCGRGHNVLRQTRVERCHCRFHWCCYVLCDECKVTEWVNVCK

SEQ ID NO: 57 - protein, 8 aa, Artificial Sequence
  Linker
ESGGGGVT

SEQ ID NO: 58 - protein, 9 aa, Artificial Sequence
  Linker
LESGGGGVT

SEQ ID NO: 59 - protein, 6 aa, Artificial Sequence
  Linker
GRAQVT

SEQ ID NO: 60 - protein, 6 aa, Artificial Sequence
  Linker
WRAQVT

SEQ ID NO: 61 - protein, 8 aa, Artificial Sequence
  Linker
ARGRAQVT

SEQ ID NO: 62 - DNA, 20 bp, Artificial Sequence
  CCND2 Forward Primer
gctgtctctgatccgcaagc

SEQ ID NO: 63 - DNA, 21 bp, Artificial Sequence
  CCND2 Reverse Primer
gacggtgggtacatggcaaac

SEQ ID NO: 64 - DNA, 26 bp, Artificial Sequence
  CCND2 Probe
ccttcattgctctgtgtgccaccgac

SEQ ID NO: 65 - DNA, 19 bp, Artificial Sequence
  CTBP2 Reverse Primer
ctcgaactgcaaccgcctg

SEQ ID NO: 66 - DNA, 24 bp, Artificial Sequence
  CTBP2 Probe
cccgtgcgaccaaagccaatgagg

SEQ ID NO: 67 - DNA, 24 bp, Artificial Sequence
  DKK1 Forward Primer
gaccattgacaactaccagccgta

SEQ ID NO: 68 - DNA, 22 bp, Artificial Sequence
  DKK1 Reverse Primer
tgggactagcgcagtactcatc

SEQ ID NO: 69 - DNA, 21 bp, Artificial Sequence
  DKK1 Probe
tgccgcactcctcgtcctctg

SEQ ID NO: 70 - DNA, 25 bp, Artificial Sequence
  FBXW2 Forward Primer
gccagttatgatattctcagggtca

SEQ ID NO: 71 - DNA, 23 bp, Artificial Sequence
  FBXW2 Reverse Primer
agcagggcaaagatatctccaaa

SEQ ID NO: 72 - DNA, 27 bp, Artificial Sequence
  FBXW2 Probe
agactcctgagatagcaaacttggcct

SEQ ID NO: 73 - DNA, 22 bp, Artificial Sequence
  RHOU1 Forward Primer
cccaccgagtacatccctactg

SEQ ID NO: 74 - DNA, 24 bp, Artificial Sequence
  RHOU1 Reverse Primer
cagtgtcacagagttggagtctca

SEQ ID NO: 75 - DNA, 22 bp, Artificial Sequence
  RHOU1 Probe
cgcccatccacagacaccaccg

SEQ ID NO: 76 - DNA, 23 bp, Artificial Sequence
  WIF1 Forward Primer
gttccaaaggttaccagggagac

SEQ ID NO: 77 - DNA, 21 bp, Artificial Sequence
  WIF1 Reverse Primer
gttgggttcatggcaggttcc

SEQ ID NO: 78 - DNA, 25 bp, Artificial Sequence
  WIF1 Probe
ccaggctcgcagacaggctttgaac

SEQ ID NO: 79 - DNA, 18 bp, Artificial Sequence
  CTBP2 Forward Primer
atccgtggggagacgctg